(19) United States
(12) Patent Application Publication
(10) Pub. No.: US 2026/0286001 A1
(43) Pub. Date: Sep. 24, 2026

(54) SUBCUTANEOUS FORMULATIONS OF THERAPEUTIC AGENTS

(71) Applicants: TG THERAPEUTICS, INC., New York, NY (US); LABORATOIRE FRANCAIS DU FRACTIONNEMENT ET DES BIOTECHNOLOGIES, Les Ulis (FR)

(72) Inventors: Aaron Springer Mayo, Arlington, TX (US); Sri Hari Pandurang Miskin, Miami Beach, FL (US); Michael S. Weiss, Boca Raton, FL (US); George Costas Avgerinos, Sudbury, MA (US)

(21) Appl. No.: 19/699,954

(22) Filed: Jun. 5, 2026

Related U.S. Application Data
(63) Continuation of application No. PCT/US26/13773, filed on Feb. 3, 2026.
(60) Provisional application No. 63/753,340, filed on Feb. 3, 2025.

(51) Int. Cl. C07K 16/28; A61K 47/18; A61K 47/26

(52) U.S. Cl. CPC C07K 16/2887; A61K 47/183; A61K 47/186; A61K 47/26

(57) ABSTRACT

Provided herein are pharmaceutical formulations comprising therapeutic agents. Also provided are methods of making and using such pharmaceutical formulations.

### CLAIM OF PRIORITY

This application is a bypass continuation and claims priority to PCT Application Serial No. PCT/US2026/013773 filed on Feb. 3, 2026, which claims the benefit of U.S. Provisional Application Ser. No. 63/753,340, filed on Feb. 3, 2025. The entire contents of the foregoing are incorporated herein by reference.

### SEQUENCE LISTING

This application contains a Sequence Listing that has been submitted electronically as an XML file named “50581-0023001_SL_ST26.XML”. The XML file, created on May 12, 2026, is 49,887 bytes in size. The material in the XML file is hereby incorporated by reference in its entirety.

### FIELD OF THE DISCLOSURE

The present disclosure is in the field of therapeutic agents, and methods of production and uses of the same.

### BACKGROUND OF THE DISCLOSURE

Therapeutic monoclonal antibodies (mAbs) are used in the treatment of a wide range of diseases, including chronic conditions such as multiple sclerosis.

### SUMMARY OF THE DISCLOSURE

Described herein are high concentration formulations of therapeutic agents for subcutaneous (SC) administration. Accordingly, described herein are pharmaceutical formulations comprising a therapeutic antibody, preferably an anti-CD20 antibody protein, in a concentration of 100-300 mg/mL. For example, described herein are pharmaceutical formulations comprising a therapeutic antibody, preferably an anti-CD20 antibody protein, in a concentration of about 200 mg/mL. Pharmaceutical formulations described herein can comprise a high concentration of the therapeutic antibody in a buffer that makes the pharmaceutical formulation suitable for subcutaneous administration. For example, the buffer can result in a viscosity of about 9-30 cP and/or an osmolality of 387 mOsm/kg to 473 mOsm/kg, thus making the pharmaceutical formulation suitable for subcutaneous administration. The buffer can comprise one or more amino acids (e.g., in their salt forms) that can act as viscosity-lowering excipients during development of the high-concentration therapeutic formulations. The buffer can comprise one or more surfactants, e.g., to inhibit aggregation of the therapeutic antibody in the formulation. The buffer can comprise one or more sugars or polyols, e.g., to stabilize the therapeutic antibody in the formulation. Thus, the buffer can comprise one or more (e.g., one, two, or all three) of the following: arginine, glutamate, and polysorbate 80.

For example, the present disclosure provides pharmaceutical formulations comprising 100-300 mg/mL of a therapeutic agent and one or more of the following: about 166 mM arginine, about 224 mM glutamate, and about 0.05% polysorbate 80.

In some embodiments, the pharmaceutical formulations further comprise sodium hydroxide.

In some embodiments, pH of the pharmaceutical formulations is about 5.5. In some embodiments, osmolality of the pharmaceutical formulations is about 430 mOsm/kg. In some embodiments, viscosity of the pharmaceutical formulations is about 9-30 cP.

In some embodiments, the pharmaceutical formulations comprise 200 mg/ml of the therapeutic agent. In some embodiments, the pharmaceutical formulations comprise the therapeutic agent in a single dosage form.

In some embodiments, the therapeutic agent is an anti-CD20 antibody protein, optionally ublituximab. In some embodiments, the anti-CD20 antibody protein comprises a heavy chain comprising the amino acid sequence of SEQ ID NO:1 and a light chain comprising the amino acid sequence of SEQ ID NO:2. In some embodiments, the anti-CD20 antibody protein comprises a heavy chain variable region (VH) comprising a VH complementarity determining region (CDR) 1 comprising the amino acid sequence of SEQ ID NO:3, a VH CDR2 comprising the amino acid sequence of SEQ ID NO:4, and a VH CDR3 comprising the amino acid sequence of SEQ ID NO:5; and a light chain variable region (VL) comprising a VL CDR1 comprising the amino acid sequence of SEQ ID NO:8, a VL CDR2 comprising the amino acid sequence of SEQ ID NO:9, and a VL CDR3 comprising the amino acid sequence of SEQ ID NO: 10. In some embodiments, the anti-CD20 antibody protein has an N-glycan profile comprising about 10 to 20% galactosylated glycans and about 20 to 40% fucosylated glycans.

Also provided herein are pharmaceutical formulations comprising: (i) a single dosage form of a therapeutic agent, wherein the therapeutic agent is present in the pharmaceutical formulation at a concentration of about 200 mg/mL, (ii) about 166 mM arginine, (iii) about 224 mM glutamate, and (iv) about 0.05% polysorbate 80.

In some embodiments, the pharmaceutical formulations further comprise sodium hydroxide.

In some embodiments, pH of the pharmaceutical formulations is about 5.5. In some embodiments, osmolality of the pharmaceutical formulations is about 430 mOsm/kg. In some embodiments, viscosity of the pharmaceutical formulations is about 9-30 cP.

In some embodiments, the therapeutic agent is an anti-CD20 antibody protein, e.g., ublituximab. In some embodiments, the anti-CD20 antibody protein comprises a heavy chain comprising the amino acid sequence of SEQ ID NO: 1 and a light chain comprising the amino acid sequence of SEQ ID NO:2. In some embodiments, the anti-CD20 antibody protein comprises a heavy chain variable region (VH) comprising a VH complementarity determining region (CDR) 1 comprising the amino acid sequence of SEQ ID NO:3, a VH CDR2 comprising the amino acid sequence of SEQ ID NO:4, and a VH CDR3 comprising the amino acid sequence of SEQ ID NO:5; and a light chain variable region (VL) comprising a VL CDR1 comprising the amino acid sequence of SEQ ID NO:8, a VL CDR2 comprising the amino acid sequence of SEQ ID NO: 9, and a VL CDR3 comprising the amino acid sequence of SEQ ID NO:10. In some embodiments, the anti-CD20 antibody protein has an N-glycan profile comprising about 10 to 20% galactosylated glycans and about 20 to 40% fucosylated glycans.

In some embodiments, the pharmaceutical formulation is stable at 2-8° C. for up to 36 months.

Also provided herein are methods of making the pharmaceutical formulation described hereinabove. The methods can comprise:
preparation of a starting material of the therapeutic agent, wherein preparation of the starting material comprises culturing a population of rat hybridoma cells in a cell culture having a culture pH of about 6.5 to about 7.55, wherein the rat hybridoma cells comprise an expression vector comprising a polynucleotide encoding the therapeutic agent; and
ultrafiltration and diafiltration of the starting material to obtain the pharmaceutical formulation, wherein the ultrafiltration and diafiltration comprises: a first ultrafiltration step; a diafiltration step in a process buffer comprising 225 mM arginine, 245 mM glutamate, and pH 5.5; a second ultrafiltration step; and formulation and dilution in a buffer comprising about 166 mM arginine, about 224 mM glutamate, and about 0.05% polysorbate 80.

In some embodiments, the preparation of the starting material comprises: i) culturing the rat hybridoma cells at a first culture pH of about 7.0 to about 7.55 for 0 to 3 days, ii) culturing the rat hybridoma cells at a second culture pH of about 6.5 to about 6.99 on day 3, iii) maintaining the culture pH at the second culture pH of about 6.5 to about 6.99 from culture day 3 until harvest of the cell culture, and iv) controlling culture pCO₂ levels to less than about 300 mmHg, optionally less than 200 mmHg, throughout the culture period.

In some embodiments, the culture pCO₂ levels is controlled to less than about 200 mmHg throughout the culture period.

In some embodiments, the second culture pH is about 6.60 to about 6.96. In some embodiments, the second culture pH is about 6.8. In some embodiments, the second culture pH results in higher integrated viable cell density (IVCD) and higher titer at harvest. In some embodiments, the second culture pH results in lower percent fucosylation.

In some embodiments, the rat hybridoma cells are cultured in a chemically defined and animal-derived component free (ADCF) culture medium.

In some embodiments, the cell culture comprises the following culture conditions: an initial temperature set point of about 37° C., wherein the initial temperature set point is set on culture day 0 to culture day 1; a second temperature set point of about 35° C., wherein the second temperature set point is set at the end of culture day 1 to culture day 3; and a third temperature set point of about 32° C. to about 33° C., optionally about 32.5° C., wherein the third temperature set point is set on culture day 3 and is maintained though harvest of the starting material.

Also provided herein are single use vials or pre-filled syringes comprising the pharmaceutical formulation described hereinabove.

In some embodiments, the single use vials or pre-filled syringes comprise the therapeutic agent in a volume of 1 ml to 5 ml. In some embodiments, the single use vials or pre-filled syringes comprise the therapeutic agents in a volume of about 2 ml.

Also provided herein are methods of treating an autoimmune disease in a human subject, by subcutaneously administering to the human subject a pharmaceutical formulation described herein.

In some embodiments, the autoimmune disease is multiple sclerosis (MS). In some embodiments, the MS is a relapsing form of MS (RMS). In some embodiments, the RMS is selected from clinically isolated syndrome (CIS), relapsing-remitting MS (RRMS) and active secondary progressive MS SPMS). In some embodiments, the subject has an Expanded Disability Status Scale (EDSS) score of from 0 to 5.5 prior to treatment. In some embodiments, the subject is diagnosed with RMS in accordance to McDonald Criteria.

In some embodiments, the autoimmune disease is rheumatoid arthritis (RA). In some embodiments, the autoimmune disease is lupus. In some embodiments, the autoimmune disease is myasthenia gravis. In some embodiments, the autoimmune disease is chronic inflammatory demyelinating polyneuropathy (CIDP).

Also provided herein are pharmaceutical formulations comprising a concentration of 100-300 mg/mL of a therapeutic antibody, preferably an anti-CD20 antibody protein, and one, two, or all three of the following: 114 mM to 192 mM arginine, 184 mM to 242 mM glutamate, and 0.03% to 0.07% polysorbate 80. In some embodiments, the pharmaceutical formulations comprise about 166 mM arginine, about 224 mM glutamate, and about 0.05% Polysorbate 80.

In some embodiments, the pharmaceutical formulations are for subcutaneous administration.

In some embodiments, the pharmaceutical formulations comprise 127 mM to 179 mM arginine. In some embodiments, the pharmaceutical formulations comprise 166±10% mM arginine. In some embodiments, the pharmaceutical formulations comprise 194 mM to 232 mM glutamate. In some embodiments, the pharmaceutical formulations comprise 224±10% mM glutamate. In some embodiments, the pharmaceutical formulations comprise 0.045% to 0.055% polysorbate 80. In some embodiments, the pharmaceutical formulations comprise about 0.05% polysorbate 80.

In some embodiments, pH of the pharmaceutical formulations is 4.95 to 6.05. In some embodiments, pH of the pharmaceutical formulations is about 5.5. In some embodiments, the pharmaceutical formulations have an osmolality of 387 mOsm/kg to 473 mOsm/kg. In some embodiments, the pharmaceutical formulations have an osmolality of about 430 mOsm/kg. In some embodiments, the pharmaceutical formulations have a viscosity of about 9-30 cP.

In some embodiments, the pharmaceutical formulations comprise a concentration of 150-250 mg/mL of the therapeutic antibody. In some embodiments, the pharmaceutical formulations comprise a concentration of about 200 mg/mL of the therapeutic antibody. In some embodiments, the pharmaceutical formulations comprise the therapeutic antibody in a single dosage form.

In some embodiments, the therapeutic antibody is an anti-CD20 antibody protein. In some embodiments, the anti-CD20 antibody protein is ublituximab.

In some embodiments, the anti-CD20 antibody protein comprises a heavy chain variable region (VH) comprising a VH complementarity determining region (CDR) 1 comprising the amino acid sequence of SEQ ID NO: 3, a VH CDR2 comprising the amino acid sequence of SEQ ID NO: 4, and a VH CDR3 comprising the amino acid sequence of SEQ ID NO: 5; and a light chain variable region (VL) comprising a VL CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

In some embodiments, the anti-CD20 antibody protein comprises a heavy chain comprising the amino acid sequence of SEQ ID NO: 1 and a light chain comprising the amino acid sequence of SEQ ID NO:2.

In some embodiments, the anti-CD20 antibody protein has an N-glycan profile comprising about 10 to 20% galactosylated glycans and about 20 to 40% fucosylated glycans.

Also provided herein are pharmaceutical formulations comprising: (i) a single dosage form of a therapeutic antibody, preferably an anti-CD20 antibody protein, wherein the therapeutic antibody is present in the pharmaceutical formulation at a concentration of about 200 mg/mL, (ii) 114 mM to 192 mM arginine, (iii) 184 mM to 242 mM glutamate, and (iv) 0.03% to 0.07% polysorbate 80. In some embodiments, the pharmaceutical formulations comprise about 166 mM arginine, about 224 mM glutamate, and about 0.05% Polysorbate 80.

In some embodiments, the pharmaceutical formulations are for subcutaneous administration.

In some embodiments, the pharmaceutical formulations comprise 127 mM to 179 mM arginine. In some embodiments, the pharmaceutical formulations comprise 166±10% mM arginine. In some embodiments, the pharmaceutical formulations comprise 194 mM to 232 mM glutamate. In some embodiments, the pharmaceutical formulations comprise 224±10% mM glutamate. In some embodiments, the pharmaceutical formulations comprise 0.045% to 0.055% polysorbate 80. In some embodiments, the pharmaceutical formulations comprise about 0.05% polysorbate 80.

In some embodiments, pH of the pharmaceutical formulations is 4.95 to 6.05. In some embodiments, pH of the pharmaceutical formulations is about 5.5. In some embodiments, osmolality of the pharmaceutical formulations is 387 mOsm/kg to 473 mOsm/kg. In some embodiments, osmolality of the pharmaceutical formulations is about 430 mOsm/kg. In some embodiments, viscosity of the pharmaceutical formulation is about 9-30 cP.

In some embodiments, the therapeutic antibody is an anti-CD20 antibody protein. In some embodiments, the anti-CD20 antibody protein is ublituximab.

In some embodiments, the anti-CD20 antibody protein comprises a heavy chain variable region (VH) comprising a VH complementarity determining region (CDR) 1 comprising the amino acid sequence of SEQ ID NO: 3, a VH CDR2 comprising the amino acid sequence of SEQ ID NO: 4, and a VH CDR3 comprising the amino acid sequence of SEQ ID NO: 5; and a light chain variable region (VL) comprising a VL CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

In some embodiments, the anti-CD20 antibody protein comprises a heavy chain comprising the amino acid sequence of SEQ ID NO: 1 and a light chain comprising the amino acid sequence of SEQ ID NO: 2.

In some embodiments, the anti-CD20 antibody protein has an N-glycan profile comprising about 10 to 20% galactosylated glycans and about 20 to 40% fucosylated glycans.

In some embodiments, the pharmaceutical formulation is stable at 2-8° C. for up to 36 months.

Also provided herein are pharmaceutical formulations comprising 100-300 mg/ml of an anti-CD20 antibody protein, and one, two, or all three of the following: 114 mM to 192 mM arginine, 184 mM to 242 mM glutamate, and 0.03% to 0.07% polysorbate 80,
wherein the anti-CD20 antibody protein comprises a heavy chain variable region (VH) comprising a VH complementarity determining region (CDR) 1 comprising the amino acid sequence of SEQ ID NO: 3, a VH CDR2 comprising the amino acid sequence of SEQ ID NO: 4, and a VH CDR3 comprising the amino acid sequence of SEQ ID NO: 5; and a light chain variable region (VL) comprising a VL CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10,
and wherein the anti-CD20 antibody protein has an N-glycan profile comprising about 10 to 20% galactosylated glycans and about 20 to 40% fucosylated glycans.

In some embodiments, the pharmaceutical formulations are for subcutaneous administration.

In some embodiments, the pharmaceutical formulations comprise a concentration of 150-250 mg/mL of the anti-CD20 antibody protein. In some embodiments, the pharmaceutical formulations comprise a concentration of about 200 mg/mL of the anti-CD20 antibody protein. In some embodiments, the pharmaceutical formulations comprise the anti-CD20 antibody protein in a single dosage form.

Also provided here are methods of making the pharmaceutical formulations described herein, wherein the methods comprise ultrafiltration and diafiltration of a starting material comprising the therapeutic antibody to obtain the pharmaceutical formulation. In some embodiments, the ultrafiltration and diafiltration comprises: a first ultrafiltration step; a diafiltration step in a process buffer comprising about 225 mM arginine, about 245 mM glutamate, and at about pH 5.5; a second ultrafiltration step; and formulation and dilution in a buffer comprising 114 mM to 192 mM arginine, 184 mM to 242 mM glutamate, and 0.03% to 0.07% Polysorbate 80. In some embodiments, the formulation and dilution is in a buffer comprising about 166 mM arginine, about 224 mM glutamate, and about 0.05% Polysorbate 80.

Also provided here are single use vials or pre-filled syringes comprising the pharmaceutical formulations described herein.

In some embodiments, the single use vials or pre-filled syringes comprise the therapeutic antibody in a volume of 1 ml to 5 ml. In some embodiments, the single use vials or pre-filled syringes comprise the therapeutic antibody in a volume of about 2 ml. In some embodiments, the single use vials or pre-filled syringes comprise a concentration of 150-250 mg/mL of the anti-CD20 antibody protein. In some embodiments, the single use vials or pre-filled syringes comprise a concentration of about 200 mg/mL of the anti-CD20 antibody protein.

Also provided here are methods of treating an autoimmune disease or a cancer in a human subject, the methods comprising subcutaneously administering to the human subject the pharmaceutical formulations described herein.

In some embodiments, the autoimmune disease is multiple sclerosis (MS). In some embodiments, the MS is a relapsing form of MS (RMS). In some embodiments, the RMS is selected from clinically isolated syndrome (CIS), relapsing-remitting MS (RRMS) and active secondary progressive MS (SPMS). In some embodiments, the subject has an Expanded Disability Status Scale (EDSS) score of from 0 to 5.5 prior to treatment. In some embodiments, the subject is diagnosed with RMS in accordance to McDonald Criteria.

In some embodiments, the autoimmune disease is rheumatoid arthritis (RA). In some embodiments, the autoimmune disease is lupus. In some embodiments, the autoimmune disease is myasthenia gravis. In some embodiments, the autoimmune disease is chronic inflammatory demyelinating polyneuropathy (CIDP).

In some embodiments, the cancer is a B-cell lymphoproliferative disorder, e.g., a hematological cancer, e.g., lymphoma, leukemia, or myeloma. In some embodiments, the hematological cancer is selected from B-cell lymphoma, acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, and primary central nervous system lymphoma (PCNSL).

In some embodiments, the methods comprise a treatment period of at least 96 weeks.

In some embodiments, the human subject has been pre-medicated with a corticosteroid 30-60 minutes prior to administration of the pharmaceutical formulation. In some embodiments, the human subject is pre-medicated with a corticosteroid prior to a first administration and not for one or more subsequent administrations of the pharmaceutical formulation. In some embodiments, the pre-treatment dosage of the corticosteroid is about 100 mg methylprednisone or 10-20 mg dexamethasone.

In some embodiments, the human subject has been pre-medicated with an antihistamine 30-60 minutes prior to administration of the pharmaceutical formulation. In some embodiments, the pre-treatment dosage of the antihistamine is about 25 to 50 mg diphenhydramine HCl.

In some embodiments, the human subject has been pre-medicated with an antipyretic 30-60 minutes prior to administration of the pharmaceutical formulation. In some embodiments, the antipyretic is acetaminophen or an antipyretic bioequivalent thereto.

As used herein, and unless otherwise specified, the term “about” or “approximately” means an acceptable error for a particular value as determined by one of ordinary skill in the art, which depends in part on how the value is measured or determined. For the avoidance of doubt, unless otherwise defined the term “about” or “approximately” means within +/−10% of a given value or range.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a flow diagram showing the manufacturing process for generating a formulation having a high concentration (200 mg/mL) of Ublituximab.

FIGS. 2A-2B are graphs showing DSC thermograms of ublituximab high concentration (200 mg/mL) (FIG. 2A) and ublituximab IV (FIG. 2B).

FIGS. 3A-3B are graphs showing sedimentation coefficient distribution plots for ublituximab high concentration (200 mg/mL) (FIG. 3A) and ublituximab IV (FIG. 3B). LMWS=low molecular weight species; HMWS1=high molecular weight species (dimer)

FIG. 4 is a graph showing TG-1101 viscosity as a function of concentration variants 1 and 2 as compared to controls.

FIG. 5 is a graph showing TG-1101 viscosity as a function of concentration variants 3 and 4 as compared to controls.

FIG. 6 is a graph showing TG-1101 viscosity as a function of concentration variants 5 and 6 as compared to controls.

FIG. 7 is a graph showing viscosity of arginine/glutamate vs citrate formulations of TG-1101.

FIGS. 8A-8G provide stability trending graphs for SEC (FIGS. 8A-8B), iCIEF (FIGS. 8C-8E), and CGE (FIGS. 8F-8G).

FIGS. 9A-9D are chromatograms corresponding to the system suitability injections for determining Polysorbate 80 content in Ublituximab by HPLC-CAD. FIG. 9A is a blank chromatogram without blank subtraction. FIG. 9B is a blank chromatogram with blank subtraction. FIG. 9C is a System Suitability Test (SST) solution (50 μg/mL) chromatogram with blank subtraction. FIG. 9D is a test sample chromatogram with blank subtraction.

FIG. 10 is a graph showing median CD19+ B-cell counts (cells/μL) of participants treated in Part 2 of the study described in Example 16.

FIG. 11 is a pair of graphs showing bioavailability of ublituximab following subcutaneous administration in participants treated in Part 2 of the study described in Example 16.

### DETAILED DESCRIPTION OF THE DISCLOSURE

While subcutaneous (SC) administration is convenient and thus favored by patients, only a limited volume can be administered subcutaneously. Thus, high concentration formulations of therapeutic mAbs are desirable. Previously, three anti-CD20 monoclonal antibodies were formulated for subcutaneous administration, but at lower concentrations. For example, Rituximab (RITUXAN HYCELA®) for subcutaneous administration is available in units of 1400 mg per 11.7 mL or 1600 mg per 13.4 mL, at a concentration of 120 mg/mL, in combination with hyaluronidase; Ocrelizumab (OCREVUS ZUNOVO™) for subcutaneous administration is available in units of 920 mg per 23 mL, at a concentration of 40 mg/mL, in combination with hyaluronidase; and Ofatumumab (KESIMPTA®) for subcutaneous administration is available in 20 mg per 0.4 mL (50 mg/mL). Ofatumumab (KESIMPTA®) for subcutaneous administration (as described, e.g., in WO2025/078463) is administered as a once-a-month dose. This formulation was later reformulated to a higher concentration formulation (90 mg/mL) for administration once every other month. Even with the reformulation, Ofatumumab (KESIMPTA®) for subcutaneous administration is available at a maximum concentration of 90 mg/mL. However, as noted in WO 2025/078463, ideal B-cell control was not achieved when moving from the once monthly to every other month administration. As the pharmacokinetic/pharmacodynamic (PK/PD) relationship was not linear, the dosing frequency could not be reduced to half by simply doubling the dose. Instead, over 6 times the dose was needed to achieve B-cell control. Moreover, it was known in the art that high-concentration antibody formulations (≥100 mg/mL) present viscosity challenges (see, e.g., Viola et al., J Control Release. 2018 Sep. 28:286:301-314). In view of these historical difficulties, there is need for high concentration formulations of therapeutic monoclonal antibodies that would be suitable for subcutaneous administration.

Described herein are high concentration formulations of therapeutic antibodies that are suitable for subcutaneous administrations. For example, described herein are pharmaceutical formulations that comprise anti-CD20 antibodies at a high concentration, e.g., at a concentration of 100-300 mg/mL (e.g., about 200 mg/mL). The high concentration formulations of anti-CD20 antibodies of the present disclosure are suitable for subcutaneous administration. Moreover, B-cell suppression was achieved when the high concentration formulations of anti-CD20 antibodies were administered at a schedule of once every 3 months (e.g., as described herein).

The formulations of the present disclosure are based, in part, on findings that the best concentration vs. viscosity response and the best size variant and charge heterogeneity response for anti-CD20 antibody (e.g., ublituximab) formulations were achieved with lower arginine/glutamate levels at a pH of about 5.5, in contrast to previous teachings (see, e.g., Borwankar et al. Industrial & Engineering Chemistry Research 2016 55 (43), 11225-11234).

The present disclosure provides pharmaceutical formulations that comprise therapeutic agents. In some embodiments, the therapeutic agents are antibodies. Thus, described herein are pharmaceutical formulations that comprise antibodies, such as therapeutic antibodies. In some embodiments, the therapeutic agents (e.g., therapeutic antibodies) are present in the pharmaceutical formulations in high concentration. In some embodiments, the antibodies are anti-CD20 antibody proteins, e.g., ublituximab. Thus, described herein are pharmaceutical formulations that comprise anti-CD20 antibody proteins and/or populations of anti-CD20 antibody proteins. In some embodiments, the present formulations do not include hyaluronidase.

Also provided herein are populations of anti-CD20 antibody proteins with specified ranges of post-translational modifications. As used herein, “population of anti-CD20 antibody proteins” can refer to a composition of anti-CD20 antibody proteins that is being tested for the abundance of post-translational modifications. The individual anti-CD20 antibody proteins in a population can comprise similar or different post-translational modifications. In a specific embodiment, a population of anti-CD20 antibody proteins refers to all anti-CD20 antibody proteins that are present in a single dosage form. In another specific embodiment, a population of anti-CD20 antibody proteins refers to all anti-CD20 antibody proteins that are present in a single batch. In another specific embodiment, a population of anti-CD20 antibody proteins is an amount sufficient to determine whether the batch of anti-CD20 antibody proteins, when compared to a reference standard, meets or fails a predetermined acceptable range of comparison value or values.

TG-1101 (TG Therapeutics, Inc.) (also known as ublituximab, UBX, UTX, TG-1101, TGTX-1101, UTUXIN™, LFB-R603, TG20, EMAB603) is the source antibody for the anti-CD20 antibody described herein having a unique glycosylation profile that is produced by the disclosed methods.

The source antibody, TG-1101, is a monoclonal antibody that targets epitopes on CD20, e.g., IRAHT (SEQ ID NO: 37), and EPAN (SEQ ID NO: 38). See, Fox, E. et al., Mult. Scler. 27:420-429 (March 2021); Babiker et al., Expert Opin Investig Drugs 27:407-412 (2018); Cotchett, K R et al., Multiple Sclerosis and Related Disorders 49:102787 (2021); Miller et al., Blood 120: Abstract No. 2756 (2012); Deng, C. et. al., J. Clin. Oncol. 31: Abstract No. 8575 (2013). TG-1101 is also described in U.S. Pat. Nos. 9,234,045 and 9,873,745.

### Abbreviations and Conventions

The following abbreviations are used throughout this application.

| Abbreviation/symbol | Meaning |
|---|---|
| ARR | annualized relapse rate |
| CIS | clinically isolated syndrome |
| CIDP | chronic inflammatory demyelinating |
| | polyneuropathy |
| DDT | Dithiothreitol |
| EDSS | Expanded Disability Status Scale |
| GlcNAC | N-Acetylglucosamine |
| HbsAg | hepatitis B virus surface antigen |
| HBV | hepatitis B virus |
| Open square | N-Acetylglucosamine |
| Man | Mannose |
| Filled, black circle | Mannose |
| Gal | Galactose |
| Open circle | Galactose |
| Open triangle | Fucose |
| Fuc | Fucose |
| Open diamond | N-Acetylneuraminic acid |
| NANA or Neu5Ac | N-Acetylneuraminic acid |
| GPA | granulomatosis with Pollyangiitis |
| GVHD | Graft Versus Host Disease |
| HC | heavy chain |
| HILIC | hydrophilic interaction liquid chromatography |
| iCIEF | Imaged Capillary Isoelectric Focusing |
| IRAP | Independent Relapse Adjudication Panel |
| K_{D} | equilibrium dissociation constants |
| LC | light chain |
| LC-MS | liquid chromatography-coupled mass spectrometer |
| MAG | Myelin Associated Glycoprotein |
| MCNS | Minimal Change Nephrotic Syndrome |
| MG | Myasthenia Gravis |
| MPA | Microscopic Polyangiitis |
| MS | multiple sclerosis |
| PPMS | primary progressive multiple sclerosis |
| RA | Rheumatoid Arthritis |
| RMS | relapsing form of multiple sclerosis |
| RRMS | relapsing-remitting multiple sclerosis |
| SDS | sodium dodecyl sulfate |
| SPMS | secondary progressive multiple sclerosis |
| TTP | Thrombotic Thrombocytopenic Purpura |

### Primary Amino Acid Sequence of the Anti-CD20 Antibody

The anti-CD20 antibody proteins provided herein can be expressed from one or more nucleic acid sequences encoding a heavy chain comprising the amino acid sequence of SEQ ID NO: 1 and a light chain comprising the amino acid sequence of SEQ ID NO:2. The anti-CD20 antibody proteins described herein can comprise a population of anti-CD20 antibody proteins expressed from one or more nucleic acid sequences encoding a heavy chain comprising the amino acid sequence of SEQ ID NO: 1 and a light chain comprising the amino acid sequence of SEQ ID NO:2. The anti-CD20 antibody proteins described herein can comprise a population of anti-CD20 antibody proteins, wherein the anti-CD20 antibody proteins in the population comprise a heavy chain comprising the amino acid sequence of SEQ ID NO:1 and/or a light chain comprising the amino acid sequence of SEQ ID NO:2. The anti-CD20 antibody proteins described herein can comprise a population of anti-CD20 antibody proteins, wherein the anti-CD20 antibody proteins in the population comprise: a heavy chain variable region (VH) comprising a VH complementarity determining region (CDR) 1 comprising the amino acid sequence of SEQ ID NO:3, a VH CDR2 comprising the amino acid sequence of SEQ ID NO:4, and a VH CDR3 comprising the amino acid sequence of SEQ ID NO:5; and/or a light chain variable region (VL) comprising a VL CDR1 comprising the amino acid sequence of SEQ ID NO: 8, a VL CDR2 comprising the amino acid sequence of SEQ ID NO:9, and a VL CDR3 comprising the amino acid sequence of SEQ ID NO:10. Sequences are provided below in the Sequence Table.

The anti-CD20 antibody proteins (e.g., anti-CD20 antibody proteins in a population of anti-CD20 antibody proteins) provided herein can be expressed from one or more nucleic acid sequences encoding a heavy chain comprising the amino acid sequence of SEQ ID NO:1 or an amino acid sequence that is at least 90%, 91%, 92%, 93%, 94%, 95%, 96%, 97%, 98%, or at least 99% identical to SEQ ID NO:1; and encoding a light chain comprising the amino acid sequence of SEQ ID NO:2 or an amino acid sequence that is at least 90%, 91%, 92%, 93%, 94%, 95%, 96%, 97%, 98%, or at least 99% identical to SEQ ID NO:2. The anti-CD20 antibody proteins (e.g., anti-CD20 antibody proteins in a population of anti-CD20 antibody proteins) can comprise a heavy chain comprising the amino acid sequence of SEQ ID NO:1 or an amino acid sequence that is at least 90%, 91%, 92%, 93%, 94%, 95%, 96%, 97%, 98%, or at least 99% identical to SEQ ID NO:1; and/or a light chain comprising the amino acid sequence of SEQ ID NO: 2 or an amino acid sequence that is at least 90%, 91%, 92%, 93%, 94%, 95%, 96%, 97%, 98%, or at least 99% identical to SEQ ID NO:2.

The anti-CD20 antibody proteins (e.g., anti-CD20 antibody proteins in a population of anti-CD20 antibody proteins) provided herein can comprise the VH of SEQ ID NO:6 and/or the VL of SEQ ID NO:11. The anti-CD20 antibody proteins (e.g., anti-CD20 antibody proteins in a population of anti-CD20 antibody proteins) can comprise a VH comprising the amino acid sequence of SEQ ID NO:6 or an amino acid sequence that is at least 90%, 91%, 92%, 93%, 94%, 95%, 96%, 97%, 98%, or at least 99% identical to SEQ ID NO:6; and/or a VL comprising the amino acid sequence of SEQ ID NO:11 or an amino acid sequence that is at least 90%, 91%, 92%, 93%, 94%, 95%, 96%, 97%, 98%, or at least 99% identical to SEQ ID NO:11.

The anti-CD20 antibody proteins (e.g., anti-CD20 antibody proteins in a population of anti-CD20 antibody proteins) provided herein can comprise a VH comprising a VH CDR1 comprising the amino acid sequence of SEQ ID NO:3, or an amino acid sequence that is at least 90%, 91%, 92%, 93%, 94%, 95%, 96%, 97%, 98%, or at least 99% identical to SEQ ID NO:3; a VH CDR2 comprising the amino acid sequence of SEQ ID NO:4, or an amino acid sequence that is at least 90%, 91%, 92%, 93%, 94%, 95%, 96%, 97%, 98%, or at least 99% identical to SEQ ID NO: 4; and a VH CDR3 comprising the amino acid sequence of SEQ ID NO:5 or an amino acid sequence that is at least 90%, 91%, 92%, 93%, 94%, 95%, 96%, 97%, 98%, or at least 99% identical to SEQ ID NO:5. Additionally or alternatively, the anti-CD20 antibody proteins (e.g., anti-CD20 antibody proteins in a population of anti-CD20 antibody proteins) provided herein comprise a VL comprising a VL CDR1 comprising the amino acid sequence of SEQ ID NO:8 or an amino acid sequence that is at least 90%, 91%, 92%, 93%, 94%, 95%, 96%, 97%, 98%, or at least 99% identical to SEQ ID NO:8; a VL CDR2 comprising the amino acid sequence of SEQ ID NO: 9 or an amino acid sequence that is at least 90%, 91%, 92%, 93%, 94%, 95%, 96%, 97%, 98%, or at least 99% identical to SEQ ID NO:9; and a VL CDR3 comprising the amino acid sequence of SEQ ID NO:10 or an amino acid sequence that is at least 90%, 91%, 92%, 93%, 94%, 95%, 96%, 97%, 98%, or at least 99% identical to SEQ ID NO:10.

The one or more nucleic acid sequences encoding the heavy chain of the anti-CD20 antibody proteins provided herein can comprise the nucleic acid sequence of SEQ ID NO:35. The one or more nucleic acid sequence encoding the light chain of the anti-CD20 antibody proteins provided herein can comprise the nucleic acid sequence of SEQ ID NO:36.

The anti-CD20 antibody proteins (e.g., anti-CD20 antibody proteins in a population of anti-CD20 antibody proteins) provided herein can bind to the same epitope as TG-1101 (TG Therapeutics, Inc.).

The anti-CD20 antibody proteins (e.g., anti-CD20 antibody proteins in a population of anti-CD20 antibody proteins) provided herein can be chimeric immunoglobulin G1 (IgG1) anti-CD20 monoclonal antibody proteins, each comprised of a tetrameric assembly from two light chains (213 amino acids) and two heavy chains (448 amino acids).

The anti-CD20 antibody proteins (e.g., anti-CD20 antibody proteins in a population of anti-CD20 antibody proteins) provided herein are expressed from one or more nucleic acid sequences encoding a heavy chain comprising the amino acid sequence of SEQ ID NO: 1 and a light chain comprising the amino acid sequence of SEQ ID NO:2 and comprise a pyroglutamate at position 1 of light chain and/or heavy chain instead of glutamine, thus resulting in an amino acid sequence of SEQ ID NO: 13 for the heavy chain and/or an amino acid sequence of SEQ ID NO: 14 for the light chain. The anti-CD20 antibody proteins (e.g., anti-CD20 antibody proteins in a population of anti-CD20 antibody proteins) provided herein can comprise a heavy chain comprising the amino acid sequence of SEQ ID NO: 1 and a light chain comprising the amino acid sequence of SEQ ID NO:2 and comprise a pyroglutamate at position 1 of light chain and/or heavy chain instead of glutamine, thus resulting in an amino acid sequence of SEQ ID NO: 13 for the heavy chain and/or an amino acid sequence of SEQ ID NO: 14 for the light chain.

The anti-CD20 antibody proteins (e.g., anti-CD20 antibody proteins in a population of anti-CD20 antibody proteins) provided herein can be expressed from one or more nucleic acid sequences encoding a heavy chain comprising the amino acid sequence of SEQ ID NO: 1 and a light chain comprising the amino acid sequence of SEQ ID NO:2 and comprise a deletion of the C-terminal lysine at the heavy chain thus resulting in the amino acid sequence of SEQ ID NO:15. The anti-CD20 antibody proteins (e.g., anti-CD20 antibody proteins in a population of anti-CD20 antibody proteins) provided herein can comprise a heavy chain comprising the amino acid sequence of SEQ ID NO: 1 and a light chain comprising the amino acid sequence of SEQ ID NO: 2 and comprise a deletion of the C-terminal lysine at the heavy chain thus resulting in the amino acid sequence of SEQ ID NO: 15.

Also provided herein are compositions or population of anti-CD20 antibody proteins wherein at least 50%, 60%, 70%, 80%, 90%, 95%, or 98% comprises (i) a pyroglutamate at position 1 (instead of glutamine) of the heavy chain, (ii) a pyroglutamate at position 1 (instead of glutamine) of the light chain, and/or (iii) a deletion of the C-terminal lysine of the heavy chain.

The anti-CD20 antibody proteins can be expressed from one or more nucleic acid sequences encoding a light chain comprising the amino acid sequence of SEQ ID NO:16. The anti-CD20 antibody proteins (e.g., an anti-CD20 antibody protein in a population of anti-CD20 antibody proteins) can comprise a light chain comprising the amino acid sequence of SEQ ID NO: 16.

The anti-CD20 antibody protein compositions provided herein can be described by various post-translational modifications and/or by their three-dimensional conformation. The respective levels of the various post-translational modifications can be quantified by means that are well known in the art.

The anti-CD20 antibody protein compositions produced in vitro have various post-translational modifications. It is understood that each individual anti-CD20 antibody protein may have its own specific pattern of post-translational modifications. To describe the properties of a population of multiple anti-CD20 antibody proteins, the overall presence of a specific post-translational modification can be quantified. Without being bound by theory, the levels of a specific post-translational modification in a population of anti-CD20 proteins can determine the biological and clinical properties of the composition (such as a dose of a pharmaceutical formulation). Without being bound by theory, the post-translational modifications can be rendered by the expression in a rat hybridoma cell (e.g., YB2/0 cell) in cell culture, e.g., as described herein and in WO 2023/235762, the entire contents of which are incorporated herein by reference.

In certain embodiments, a type of post-translational modification that can be used to describe the anti-CD20 antibody protein compositions provided herein is glycosylation. Various glycosylations are known and are described in detail in WO 2023/235764, which is incorporated herein by reference in its entirety. For example, the population of anti-CD20 antibody proteins described herein can have a glycosylation profile as described in WO 2023/235764, which is incorporated herein by reference in its entirety. In some embodiments, the glycosylation is N-glycosylation. For example, the population of anti-CD20 antibody proteins described herein can have an N-glycan profile comprising about 10 to 20% galactosylated glycans and about 20 to 40% fucosylated glycans.

Assays for determining the amount of post-translational modifications in a population of antibody proteins (e.g., such as an anti-CD20 antibody population as described herein) are well known in the art. Assays can be used to determine the amount of one or more of asparagine deamidation, methionine oxidation, glycosylation, pyroglutamate formation, and lysine truncation. Assays are described in detail in WO 2023/235764, which is incorporated herein by reference in its entirety.

### Total Protein

The amount of total protein can be measured using methods know in the art, including spectrophotometry. For example, the amount of total protein can be measured by absorbance at 280 nm, e.g., by using methods described in WO 2023/235764, the entire contents of which are incorporated herein by reference.

The population of anti-CD20 antibody proteins in the compositions for subcutaneous administration as provided herein can have an amount of total protein of 75 mg/mL-500 mg/mL. For example, the population can have an amount of total protein of about 100 mg/mL-300 mg/mL. For example, the population can have an amount of total protein of about 75 mg/mL, about 100 mg/mL, about 125 mg/mL, about 150 mg/mL, about 175 mg/mL, about 200 mg/mL, about 225 mg/mL, about 250 mg/mL, about 275 mg/mL, about 300 mg/mL, about 325 mg/mL, about 350 mg/mL, about 375 mg/mL, about 400 mg/mL, about 425 mg/mL, about 450 mg/mL, about 475 mg/mL, or about 500 mg/mL. Preferably, the population can have an amount of total protein of about 200 mg/mL, e.g., 150-250 mg/mL.

### Methods of Making

Also provided here are methods for making the populations of anti-CD20 antibody proteins as described hereinabove. A detailed description of such methods is provided in WO 2023/235762, the entire contents of which are incorporated herein by reference.

The methods of making the populations of anti-CD20 antibody proteins with specified ranges of post-translational modifications as described herein can comprise: i) culturing the rat hybridoma cells at a first culture pH of about 7.0 to about 7.55 for 0 to 3 days, ii) culturing the rat hybridoma cells at a second culture pH of about 6.5 to about 6.99 on day 3, iii) maintaining the culture pH at the second culture pH of about 6.5 to about 6.99 from culture day 3 until day 14 of the cell culture, and iv) controlling culture pCO₂ levels to less than about 200 mmHg throughout the culture period. In some embodiments, the second culture pH is about 6.60 to about 6.96 (e.g., the second culture pH is 6.8).

The second culture pH can result in higher integrated viable cell density (IVCD) and higher titer at harvest;

The second culture pH can result in lower percent fucosylation.

The rat hybridoma cells expressing the recombinant protein can be cultured in a basal media that is chemically defined and animal-derived component free (ADCF) culture medium.

The basal medium can be supplemented with a feed medium.

The methods can further comprise an initial temperature set point of about 37° C., wherein said initial temperature set point is set on culture day 0 to culture day 1.

The methods can further comprise a second temperature set point of about 35° C., wherein said second temperature set point is set at the end of culture day 1 to culture day 3.

The methods can further comprise a third temperature set point of about 32° C. to about 33° C., wherein said third temperature set point is set on culture day 3 and is maintained though harvest. The term “harvest” refers to the point of time in the mammalian cell culture process when cells containing the recombinant protein are separated and removed from the cell culture media and subject to additional processing, such as, e.g., centrifugation, filtration, or purification.

The harvest of the cells can occur on process day 12, 13, or 14 of the cell culture, or when cell viability drops below 20%, whichever comes first.

The methods can further comprise harvesting the recombinant protein produced by the rat hybridoma cell.

The methods can further comprise purifying the recombinant protein by affinity chromatography and/or ion exchange chromatography; as one example, the affinity chromatography can comprise protein A purification.

The methods can result in an increased yield of recombinant protein. For example, the recombinant protein can be increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to a recombinant protein produced by a culturing process that does not employ the culture conditions as recited above.

Also described herein are methods of making pharmaceutical formulations as described herein. Thus, pharmaceutical formulations comprising high concentration, such as about 100 mg/mL-300 mg/mL (e.g., about 100 mg/mL, 150 mg/mL, 200 mg/mL, 250 mg/mL, or 300 mg/mL) of a therapeutic agent (e.g., an anti-CD20 antibody as described above) can be produced by the methods described here. For example, described herein are methods of making pharmaceutical formulations comprising about 100 mg/mL-300 mg/mL (e.g., about 100 mg/mL, 150 mg/mL, 200 mg/mL, 250 mg/mL, or 300 mg/mL) of an anti-CD20 antibody of the present disclosure. In some embodiments, a therapeutic agent starting material is prepared, from which a high concentration therapeutic agent is further processed. For example, anti-CD20 antibody produced by methods described in WO 2023/235762 (the entire contents of which are incorporated herein by reference) can be further processed to produce pharmaceutical formulations comprising high concentrations, such as 100 mg/mL-300 mg/mL (e.g., about 100 mg/mL, 150 mg/mL, 200 mg/mL, 250 mg/mL, or 300 mg/mL) of the anti-CD20 antibody. Detailed description of methods for preparing such pharmaceutical formulations is provided herein, e.g., in the Examples.

Briefly, anti-CD20 antibody proteins with specified ranges of post-translational modifications as described above can be produced in rat hybridoma cells that comprise an expression vector comprising a polynucleotide encoding the antibody protein. The anti-CD20 antibody proteins can be produced in the rat hybridoma cells by culturing the rat hybridoma cells in a cell culture having a culture pH of about 6.5 to about 7.55.

The anti-CD20 antibody proteins with specified ranges of post-translational modifications can be produced from the rat hybridoma cells by: i) culturing the rat hybridoma cells at a first culture pH of about 7.0 to about 7.55 for 0 to 3 days (e.g., on day 0, day 1, day 2, and day 3 of the cell culture), ii) culturing the rat hybridoma cells at a second culture pH of about 6.5 to about 6.99 on day 3, iii) maintaining the culture pH at the second culture pH of about 6.5 to about 6.99 from culture day 3 until harvest of the cell culture (e.g., from culture day 3 until day 14 of the cell culture), and iv) controlling culture pCO₂ levels to less than about 300 mmHg (e.g., less than about 200 mmHg) throughout the culture period. pCO₂ levels less than about 300 mmHg can be facilitated by supplementing the cell culture with additional buffers, increasing the air sparge rate, increasing the dissolved oxygen (DO) setpoint, and/or decreasing the agitation rate. The second culture pH can be about 6.60 to about 6.96 (e.g., the second culture pH is 6.8). The second culture pH can result in higher integrated viable cell density (IVCD) and higher titer at harvest. The second culture pH can result in lower percent fucosylation. The harvest titer of the antibody protein can be increased and/or fucosylation of the antibody protein can be decreased when the culture pH is 6.6 to 6.96 relative to a cell culture under the same culture conditions except that the culture pH is 6.60 to 6.8. The rat hybridoma cells can be cultured in a basal media that is chemically defined and animal-derived component free (ADCF) culture medium. The basal medium can be supplemented with a feed medium. The methods can further comprise an initial temperature set point of about 37° C. For example, the initial temperature set point can be set on culture day 0 to culture day 1. The methods can further comprise a second temperature set point of about 35° C., e.g., set at the end of culture day 1 to culture day 3. The methods can further comprise a third temperature set point of about 32° C. to about 33° C., e.g., a third temperature set point that can be set on culture day 3 and maintained though harvest. The term harvest can refer to the point of time in the mammalian cell culture process when cells containing the recombinant protein are separated and removed from the cell culture media and subject to additional processing, such as, e.g., centrifugation, filtration, or purification. For example, harvest of the cells can occur on process day 12, 13, or 14 of the cell culture, or when cell viability drops below 20%, whichever comes first.

In some embodiments, the anti-CD20 antibody proteins with specified ranges of post-translational modifications are produced from the rat hybridoma cells by culturing the rat hybridoma cells in a cell culture having a culture pH of about 6.5 to about 7.55. In some embodiments, the culture pH is about 6.5 to about 7.0. In certain embodiments, the culture pH of about 6.5 to about 7.0 is set on culture day 2 of the cell culture. In certain embodiments, the culture pH of about 6.5 to about 7.0 is set on culture day 3 of the cell culture. In some embodiments, the culture pH is about 7.0 to about 7.55. In certain embodiments, the culture pH of about 7.0 to about 7.55 is set on culture days 0 to 3 of the cell culture. In some embodiments, the culture pH is lowered on day 2 or day 3 of the cell culture to about 6.5 to about 7.0. In certain embodiments, the culture pH is lowered on day 3 of the cell culture. In some embodiments, the culture pH of about 6.5 to about 7.0 is maintained from culture day 3 until harvest of the cell culture. In some embodiments, the cumulative culture time that pH is allowed to drop below a fixed pH setpoint and the cumulative magnitude of the drop, after the pH is lowered on day 3 (integrated pH2 difference), is less, relative to a cell culture with an integrated pH2 difference that is more. In certain embodiments, the fixed pH setpoint is pH 6.91. In some embodiments, lower integrated pH2 difference results in higher integrated viable cell density (IVCD) and higher titer at harvest. In some embodiments, lower integrated pH2 difference further results in lower percent fucosylation. In some embodiments, the rat hybridoma cells expressing the antibody protein are cultured in a chemically defined and animal-derived component free (ADCF) culture medium. In some embodiments, harvest titer of the antibody protein is increased and/or fucosylation of the antibody protein is decreased when the culture pH is 6.6 to 6.96 relative to a cell culture under the same culture conditions except that the culture pH is 6.60 to 6.8. In some embodiments, the method further comprises controlling culture pCO₂ levels to less than about 300 mmHg. For example, pCO₂ levels less than about 300 mmHg can be facilitated by supplementing the cell culture with additional buffers, increasing the air sparge rate, increasing the dissolved oxygen (DO) setpoint, and/or decreasing the agitation rate. In some embodiments, the method further comprises an initial temperature set point of about 37° C. In certain embodiments, the initial temperature set point is set on culture day 0 to culture day 1. In some embodiments, the method further comprises a second temperature set point of about 35° C. In certain embodiments, the second temperature set point is set at the end of culture day 1 to culture day 3. In some embodiments, the end of culture day one is 17 to 33 hours after the start of the cell culture. In some embodiments, the method further comprises a third temperature set point of about 32° C. to about 33° C. In certain embodiments, the third temperature set point is set on culture day 3 and is maintained though harvest. In certain embodiments, the third temperature set point is 32.5° C.

The cell culture can comprise the following culture conditions:
i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set on culture day 3 and is maintained though harvest;
ii) a culture pH between about 6.5 to about 7.55; and
iii) a culture pCO₂ less than about 300 mmHg.

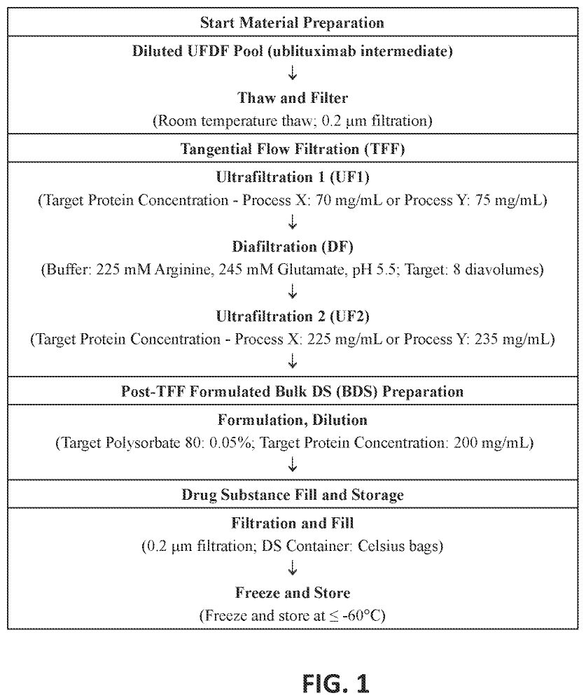

An anti-CD20 antibody protein produced by the methods described herein (can be referred to herein as the starting material) can be further processed to produce higher concentration (e.g., 100 mg/mL-300 mg/mL, such as 200 mg/mL) of the anti-CD20 antibody protein. An exemplary manufacturing process for a high concentration ublituximab formulation (200 mg/mL) from a starting material is illustrated in FIG. 1. Production of higher concentration of the anti-CD20 antibody protein from the starting material can involve an ultrafiltration and diafiltration (UFDF) process. The UFDF process can use a tangential flow filtration (TFF) system (e.g., with 30 kDa nominal molecular weight cut-off (NMWCO) membranes). Production of a higher concentration of the anti-CD20 antibody protein from the starting material can include one or more ultrafiltration steps to concentrate the starting material. The ultrafiltration step (e.g., a first ultrafiltration step or UF1) can be followed by a diafiltration step to exchange the product into a formulation buffer (without PS80). The process buffer for diafiltration can comprise 225 mM arginine and/or 245 mM glutamate. The process buffer for diafiltration can have a pH of around 5.5. After diafiltration, the product can be concentrated via another ultrafiltration step (e.g., a second ultrafiltration step or UF2) and recovered from the TFF system. After UFDF recovery, the product can be diluted with Polysorbate 80 (PS80 (e.g., 0.05% PS80) to the desired product concentration to obtain high concentration (e.g., 100 mg/mL-300 mg/mL, such as 200 mg/mL) of the anti-CD20 antibody protein. Thus, the high concentration (e.g., 100 mg/mL-300 mg/mL, such as 200 mg/mL) anti-CD20 antibody protein can be formulated in a formulation buffer comprising 114 mM to 192 mM (e.g., approximately 166=10% mM) arginine, 184 mM to 242 mM (e.g., approximately 224±10% mM) glutamate, and 0.03% to 0.07% (e.g., 0.045% to 0.055% or approximately 0.05%) polysorbate 80, pH 4.95 to 6.05 (e.g., pH 5.5). The excipient concentrations in the high concentration anti-CD20 antibody protein solution can vary from the nominal values due to the Gibbs-Donnan effect (Kannan et al., 2023 (Journal of Pharmaceutical Sciences 112 (2023) 820-829)). As described in Table 22A, the excipient concentrations in the solution can be approximately 166-8 mM (e.g., 166 mM) arginine and 224±7 mM (e.g., 224 mM) glutamate. The high concentration anti-CD20 antibody protein can be filtered (e.g., 0.2 μm filtration) and frozen and stored at ≤−60° C.

### Pharmaceutical Formulations

Also described herein are pharmaceutical formulations suitable for subcutaneous administration. The pharmaceutical formulation can comprise a therapeutic agent (e.g., an anti-CD20 antibody as described above) and one or more excipients (e.g., one or more of the following: L-arginine, L-glutamate, and polysorbate 80). The pharmaceutical formulation can further comprise sodium hydroxide. The sodium hydroxide can be used for adjusting pH of the pharmaceutical formulation (e.g., for adjusting pH of the pharmaceutical formulation to about 4.95 to about 6.05 (e.g., about pH 5.5)). The pharmaceutical formulation can comprise high concentration of a therapeutic agent (e.g., high concentration of an anti-CD20 antibody described herein). For example, provided herein are pharmaceutical formulations comprising anti-CD20 antibody proteins (or a population thereof) at a concentration of about 100 mg/ml to about 300 mg/ml (e.g., about 200 mg/ml).

### Therapeutic Agents

In some embodiments, the pharmaceutical formulation comprises a therapeutic agent, such as an antibody. In some embodiments, the antibody is an anti-CD20 antibody protein composition, such as the one described above. For example, provided herein are pharmaceutical formulations comprising anti-CD20 antibody proteins (or a population thereof), wherein the anti-CD20 antibody protein (e.g., in the population) comprises a heavy chain comprising the amino acid sequence of SEQ ID NO: 1 and a light chain comprising the amino acid sequence of SEQ ID NO: 2. Further provided herein are pharmaceutical formulation comprising anti-CD20 antibody proteins (or a population thereof), wherein the anti-CD20 antibody proteins in the population comprise: a VH comprising a VH CDR1 comprising the amino acid sequence of SEQ ID NO:3, a VH CDR2 comprising the amino acid sequence of SEQ ID NO:4, and a VH CDR3 comprising the amino acid sequence of SEQ ID NO:5; and/or a VL comprising a VL CDR1 comprising the amino acid sequence of SEQ ID NO:8, a VL CDR2 comprising the amino acid sequence of SEQ ID NO: 9, and a VL CDR3 comprising the amino acid sequence of SEQ ID NO:10.

In the pharmaceutical formulations described herein, the anti-CD20 antibody protein composition can be present in the pharmaceutical formulations at a concentration of about 75 mg/ml to 500 mg/ml (e.g., about 100 mg/ml to about 500 mg/ml; about 125 mg/ml to about 500 mg/ml; about 150 mg/ml to about 500 mg/ml; about 175 mg/ml to about 500 mg/ml; about 200 mg/ml to about 500 mg/ml; about 225 mg/ml to about 500 mg/ml; about 250 mg/ml to about 500 mg/ml; about 275 mg/ml to about 500 mg/ml; about 300 mg/ml to about 500 mg/ml; about 325 mg/ml to about 500 mg/ml; about 350 mg/ml to about 500 mg/ml; about 375 mg/ml to about 500 mg/ml; about 400 mg/ml to about 500 mg/ml; about 425 mg/ml to about 500 mg/ml; about 450 mg/ml to about 500 mg/ml; about 475 mg/ml to about 500 mg/ml; about 75 mg/ml to about 400 mg/ml; about 100 mg/ml to about 300 mg/ml; about 150 mg/ml to about 250 mg/ml; about 175 mg/ml to about 225 mg/ml; about 50 mg/ml to about 150 mg/ml; about 75 mg/ml to about 175 mg/ml; about 100 mg/ml to about 200 mg/ml; about 125 mg/ml to about 225 mg/ml; about 150 mg/ml to about 250 mg/ml; about 175 mg/ml to about 275 mg/ml; about 200 mg/ml to about 300 mg/ml; about 225 mg/ml to about 325 mg/ml; about 250 mg/ml to about 350 mg/ml; about 275 mg/ml to about 375 mg/ml; about 300 mg/ml to about 400 mg/ml; about 325 mg/ml to about 425 mg/ml; about 350 mg/ml to about 450 mg/ml; about 375 mg/ml to about 475 mg/ml; or about 400 mg/ml to about 500 mg/ml (e.g., about 75 mg/ml; about 100 mg/ml; about 125 mg/ml; about 150 mg/ml; about 175 mg/ml; about 200 mg/ml; about 225 mg/ml; about 250 mg/ml; about 275 mg/ml; about 300 mg/ml; about 325 mg/ml; about 350 mg/ml; about 375 mg/ml; about 400 mg/ml; about 425 mg/ml; about 450 mg/ml; about 475 mg/ml; or about 500 mg/ml)). For example, provided herein is a pharmaceutical formulation comprising anti-CD20 antibody proteins (or a population thereof) at a concentration of about 100 mg/ml to about 300 mg/ml (e.g., about 200 mg/ml).

### Arginine

The pharmaceutical formulations of the present disclosure can include arginine (e.g., L-arginine). L-arginine can be included in the pharmaceutical formulation at about 50 mM to about 350 mM. For example, pharmaceutical formulations described herein can include arginine (e.g., L-arginine) in a concentration of about 75 mM to about 350 mM, about 100 mM to about 350 mM, about 125 mM to about 350 mM, about 150 mM to about 350 mM, about 175 mM to about 350 mM, about 200 mM to about 350 mM, about 225 mM to about 350 mM, about 250 mM to about 350 mM, about 275 mM to about 350 mM, about 300 mM to about 350 mM, or about 325 mM to about 350 mM, about 75 mM to about 325 mM, about 100 mM to about 300 mM, about 125 mM to about 275 mM, about 150 mM to about 250 mM, about 175 mM to about 200 mM, about 50 mM to about 100 mM, about 75 mM to about 125 mM, about 100 mM to about 150 mM, about 125 mM to about 175 mM, about 150 mM to about 200 mM, about 175 mM to about 225 mM, about 200 mM to about 250 mM, about 225 mM to about 275 mM, about 250 mM to about 300 mM, about 275 mM to about 325 mM, or about 300 mM to about 350 mM (e.g., about 50 mM, about 75 mM, about 100 mM, about 125 mM, about 150 mM, about 175 mM, about 200 mM, about 225 mM, about 250 mM, about 275 mM, about 300, about 325 mM, or about 350 mM), or any integer in between. In some instances, pharmaceutical formulations described herein comprise arginine (e.g., L-arginine) in a concentration of 100 mM to 200 mM, 114 mM to 192 mM, 115 mM to 190 mM, 120 mM to 185 mM, 127 mM to 179 mM, 130 mM to 170 mM, 140 mM to 166 mM, or 150 mM to 160 mM, or any integer in between. In some instances, pharmaceutical formulations described herein comprise 114 mM to 192 mM arginine, e.g., as described in Table 22B and Table 22C hereinbelow. In some instances, pharmaceutical formulations described herein comprise 127 mM to 179 mM arginine, e.g., as described in Table 22B and Table 22C hereinbelow. In some instances, pharmaceutical formulations described herein comprise arginine (e.g., L-arginine) in a concentration of 166±10% mM (e.g., 149 mM to 182 mM, 150 mM to 180 mM, and/or 160 mM to 170 mM). Also described herein are pharmaceutical formulations comprising about 159 mM to about 182 mM arginine (e.g., L-arginine). Also described herein are pharmaceutical formulations comprising about 156 mM to about 176 mM arginine (e.g., L-arginine). Also described herein are pharmaceutical formulations comprising about 166±8 mM arginine (e.g., L-arginine), e.g., as described in Table 22A hereinbelow. For example, pharmaceutical formulations described herein can comprise about 166 mM arginine (e.g., L-arginine).

### Glutamate

The pharmaceutical formulations of the present disclosure can include glutamate (e.g., glutamic acid, such as L-glutamic acid). Glutamate can be included in the pharmaceutical formulation at about 150 mM to about 450 mM. For example, pharmaceutical formulations described herein can comprise glutamate (e.g., L-glutamic acid) in a concentration of about 175 mM to about 450 mM, about 200 mM to about 450 mM, about 225 mM to about 450 mM, about 250 mM to about 450 mM, about 275 mM to about 450 mM, about 300 mM to about 450 mM, about 325 mM to about 450 mM, about 350 mM to about 450 mM, about 375 mM to about 450 mM, about 400 mM to about 450 mM, or about 425 mM to about 450 mM, about 175 mM to about 425 mM, about 200 mM to about 400 mM, about 225 mM to about 375 mM, about 250 mM to about 350 mM, or about 275 mM to about 300 mM, about 150 mM to about 200 mM, about 175 mM to about 225 mM, about 200 mM to about 250 mM, about 225 mM to about 275 mM, about 250 mM to about 300 mM, about 275 mM to about 325 mM, about 300 mM to about 350 mM, about 325 mM to about 375 mM, about 350 mM to about 400 mM, about 375 mM to about 425 mM, or about 400 mM to about 450 mM (e.g., about 150 mM, about 175 mM, about 200 mM, about 225 mM, about 245 mM, about 250 mM, about 275 mM, about 300 mM, about 325 mM, about 350 mM, about 375 mM, about 400, about 425 mM, or about 450 mM), or any integer in between. In some instances, pharmaceutical formulations described herein comprises glutamate (e.g., L-glutamic acid) in a concentration of 180 mM to 250 mM, 184 mM to 242 mM, 190 mM to 240 mM, 194 mM to 232 mM, 200 mM to 230 mM, or 203 mM to 223 mM, or any integer in between. In some instances, pharmaceutical formulations described herein comprise 184 mM to 242 mM glutamate, e.g., as described in Table 22B and Table 22C hereinbelow. In some instances, pharmaceutical formulations described herein comprise 194 mM to 232 mM glutamate, e.g., as described in Table 22B and Table 22C hereinbelow. In some instances, pharmaceutical formulations described herein comprise glutamate (e.g., L-glutamic acid) in a concentration of about 224±10% mM (e.g., 201 mM to about 246 mM, 210 mM to 240 mM, and/or 220 mM to 230 mM). Also described herein are pharmaceutical formulations comprising about 214 mM to about 234 mM glutamate (e.g., L-glutamic acid). Also described herein are pharmaceutical formulations comprising about 218 mM to about 237 mM glutamate (e.g., L-glutamic acid). Also described herein are pharmaceutical formulations comprising about 224±7 mM glutamate (e.g., L-glutamic acid), e.g., as described in Table 22A herein. For example, pharmaceutical formulations described herein can comprise about 224 mM glutamate (e.g., L-glutamic acid).

### Polysorbate 80

The pharmaceutical formulations of the present disclosure can include an emulsifier such as polysorbate 80 (PS80). The pharmaceutical formulations described herein can include about 0.01% to about 0.1% polysorbate 80. For example, pharmaceutical formulations described herein can comprise polysorbate 80 in a concentration of about 0.015% to about 0.1%, about 0.02% to about 0.1%, about 0.025% to about 0.1%, about 0.03% to about 0.1%, about 0.035% to about 0.1%, about 0.04% to about 0.1%, about 0.045% to about 0.1%, about 0.05% to about 0.1%, about 0.055% to about 0.1%, about 0.06% to about 0.1%, about 0.065% to about 0.1%, about 0.07% to about 0.1%, about 0.075% to about 0.1%, about 0.08% to about 0.1%, about 0.085% to about 0.1%, about 0.09% to about 0.1%, or about 0.095% to about 0.1%, about 0.015% to about 0.095%, about 0.02% to about 0.09%, about 0.025% to about 0.085%, about 0.03% to about 0.08%, about 0.035% to about 0.075%, 0.03% to about 0.07%, about 0.04% to about 0.07%, about 0.045% to about 0.065%, or about 0.05% to about 0.06%, about 0.01% to about 0.02%, about 0.015% to about 0.025%, about 0.02% to about 0.03%, about 0.025% to about 0.035%, about 0.03% to about 0.04%, about 0.035% to about 0.045%, about 0.04% to about 0.05%, about 0.045% to about 0.055%, about 0.05% to about 0.06%, about 0.055% to about 0.065%, about 0.06% to about 0.07%, about 0.065% to about 0.075%, about 0.07% to about 0.08%, about 0.075% to about 0.085%, about 0.08% to about 0.09%, about 0.085% to about 0.095%, or about 0.09% to about 0.1% (e.g., about 0.01%, about 0.015%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, or about 0.1%), or any integer in between. Thus, described herein are pharmaceutical formulations comprising polysorbate 80 in a concentration of 0.03% to 0.07%, 0.035% to 0.065%, 0.04% to 0.06%, and/or 0.045% to 0.055%. For example, pharmaceutical formulations described herein can comprise about 0.05% polysorbate 80. Also described herein are pharmaceutical formulations comprising polysorbate 80 in a concentration of 0.045% to 0.055%, 0.047% to 0.053%, and/or 0.049% to 0.051%.

### Sodium Hydroxide

The pharmaceutical formulations can comprise sodium hydroxide. The sodium hydroxide can be used for adjusting pH of the pharmaceutical formulations (e.g., for adjusting pH of the pharmaceutical formulation to about 4.95 to about 6.05 (e.g., about pH 5.5)).

pH

The pharmaceutical formulations of the present disclosure can have pH of about 4.5 to about 6.5. For example, pharmaceutical formulations described herein can have pH of about 4.75 to about 6.5, about 5.0 to about 6.5, about 5.25 to about 6.5, about 5.5 to about 6.5, about 5.75 to about 6.5, about 6.0 to about 6.5, about 6.25 to about 6.5, about 4.75 to about 6.25, about 5.0 to about 6.0, or about 5.25 to about 5.75, about 4.5 to about 5.5, about 5.0 to about 6.0, or about 5.5 to about 6.5 (e.g., about 4.5, about 4.75, about 5.0, about 5.25, about 5.5, about 5.75, about 6.0, about 6.25, or about 6.5), or any integer in between. For example, pharmaceutical formulations described herein can have pH of about 5.5. Also described herein are pharmaceutical formulations having a pH of 4.95 to 6.05, a pH of 5.0 to 6.0, and/or a pH of 5.3 to 5.7.

### Osmolality

The pharmaceutical formulations of the present disclosure can have osmolality of about 300 mOsm/kg to about 600 mOsm/kg. For example, pharmaceutical formulations described herein can have osmolality of about 325 mOsm/kg to about 600 mOsm/kg, about 350 mOsm/kg to about 600 mOsm/kg, about 375 mOsm/kg to about 600 mOsm/kg, about 400 mOsm/kg to about 600 mOsm/kg, about 425 mOsm/kg to about 600 mOsm/kg, about 450 mOsm/kg to about 600 mOsm/kg, about 475 mOsm/kg to about 600 mOsm/kg, about 500 mOsm/kg to about 600 mOsm/kg, about 525 mOsm/kg to about 600 mOsm/kg, about 550 mOsm/kg to about 600 mOsm/kg, about 575 mOsm/kg to about 600 mOsm/kg, about 325 mOsm/kg to about 575 mOsm/kg, about 350 mOsm/kg to about 550 mOsm/kg, about 375 mOsm/kg to about 525 mOsm/kg, about 400 mOsm/kg to about 500 mOsm/kg, about 425 mOsm/kg to about 475 mOsm/kg, about 300 mOsm/kg to about 350 mOsm/kg, about 325 mOsm/kg to about 375 mOsm/kg, about 350 mOsm/kg to about 400 mOsm/kg, about 375 mOsm/kg to about 425 mOsm/kg, about 400 mOsm/kg to about 450 mOsm/kg, about 425 mOsm/kg to about 475 mOsm/kg, about 450 mOsm/kg to about 500 mOsm/kg, about 475 mOsm/kg to about 525 mOsm/kg, about 500 mOsm/kg to about 550 mOsm/kg, about 525 mOsm/kg to about 575 mOsm/kg, or about 550 mOsm/kg to about 600 mOsm/kg (e.g., about 300 mOsm/kg, about 325 mOsm/kg, about 350 mOsm/kg, about 375 mOsm/kg, about 400 mOsm/kg, about 425 mOsm/kg, about 450 mOsm/kg, about 475 mOsm/kg, about 500 mOsm/kg, about 525 mOsm/kg, about 550 mOsm/kg, or about 600 mOsm/kg). For example, pharmaceutical formulations described herein can have osmolality of about 430 mOsm/kg. Also described herein are pharmaceutical formulations having an osmolality of 387 mOsm/kg to 473 mOsm/kg, 390 mOsm/kg to 470 mOsm/kg, 400 mOsm/kg to 460 mOsm/kg, 410 mOsm/kg to 450 mOsm/kg, and/or 420 mOsm/kg to 440 mOsm/kg.

### Viscosity

The pharmaceutical formulations of the present disclosure can have a viscosity of about 1-50 cP. For example, pharmaceutical formulations described herein can have a viscosity of about 10-50 cP, about 20-50 cP, about 30-50 cP, about 5-45 cP, about 10-40 cP, about 15-35 cP, about 20-30 cP, about 1-10 cP, about 5-15 cP, about 10-20 cP, about 15-25 cP, about 20-30 cP, about 25-35 cP, about 30-40 cP, about 35-45 cP, or about 40-50 cP. For example, pharmaceutical formulations described herein can have a viscosity of about 9-30 cp.

### Pharmaceutical Formulation for Subcutaneous Injection

In the methods of the present disclosure, the pharmaceutical formulations can be administered by subcutaneous injection to a subject in need thereof. For example, a pharmaceutical formulation comprising a therapeutic agent (e.g., an anti-CD20 antibody protein or a population thereof) at a concentration of about 200 mg/ml is administered by subcutaneous injection to a subject in need thereof. The pharmaceutical formulation that is administered by subcutaneous injection comprises one, two, three, or more of the following excipients: L-arginine, L-glutamic acid, polysorbate 80, and sodium hydroxide. The pharmaceutical formulation that is administered by subcutaneous injection can comprise one or more of the following: 114 mM to 192 mM (e.g., 166±10% mM) arginine (e.g., L-arginine), 184 mM to 242 mM (e.g., 224±10% mM) glutamate (e.g., L-glutamic acid), and 0.03% to 0.07% (e.g., 0.045% to 0.055% or about 0.05%) polysorbate 80. In some instances, the pharmaceutical formulation that is administered by subcutaneous injection comprises sodium hydroxide. The sodium hydroxide can be used for adjusting pH of the pharmaceutical formulation (e.g., for adjusting pH of the pharmaceutical formulation to 4.95 to 6.05 (e.g., about pH 5.5)). In some instances, the pharmaceutical formulation that is administered by subcutaneous injection comprises water. The water can be used for adjusting the volume of the pharmaceutical formulation. For example, water can be used to adjust the volume of the pharmaceutical formulation to about 2 ml (e.g., in a single use vial and/or in a pre-filled syringe).

### Stability of Therapeutic Agents

Stability of therapeutic agents in the pharmaceutical formulations of the present disclosure can be assessed by methods described herein. Detailed description of such methods is provided in Example 10 hereinbelow. For example, stability of pharmaceutical compositions comprising anti-CD20 antibody proteins described herein can be assessed by the methods of the present disclosure.

The stability of pharmaceutical formulations comprising the therapeutic agents (e.g., anti-CD20 antibody proteins) can be assessed by observing one or more of the following characteristics/properties over a period of time: appearance (e.g., by observing opalescence and/or color), pH, osmolality, protein concentration, container closure integrity testing (CCIT), sterility, visible particulates, particulate matter, Size Exclusion Chromatography (SEC) (e.g., by observing % monomer, % total aggregates (e.g., dimer+aggregates), and/or % fragments), iCIEF (e.g., by observing % charged species (e.g., % acidic species and/or % basic species)), CGE (reduced and non-reduced), Polysorbate 80 content, ADCC activity, and/or CDC activity. In some embodiments, a minimum rate of change in one or more of these criteria of a therapeutic agent (e.g., an anti-CD20 antibody protein) under an intended storage condition of 2-8° C. (5±3° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) demonstrate the stability of the therapeutic agent for the intended storage condition. Additionally, or in the alternative, a minimum rate of change in one or more of these criteria of a therapeutic agent (e.g., an anti-CD20 antibody protein) under an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for an intended storage condition of 2-8° C. (5±3° C.).

For example, a minimum rate of change in appearance (e.g., as assessed by opalescence and/or color) of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in pH of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in osmolality of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in protein concentration of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in CCIT of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in sterility of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in visible particulates in a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in particulate matter in a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in SEC (e.g., showing % monomer, % total aggregates (e.g., dimer+aggregates), and/or % fragments) of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in iCIEF (e.g., showing % charged species (e.g., % acidic species and/or % basic species)) of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in CGE (e.g., reduced and/or non-reduced) of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in Polysorbate 80 content of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in ADCC activity of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

Additionally, or in the alternative, a minimum rate of change in CDC activity of a therapeutic agent (e.g., an anti-CD20 antibody protein) under an intended storage condition of 2-8° C. (5±3° C.) and/or an accelerated condition of 25° C. (25±2° C.) over a period of time (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more) can demonstrate the stability of the therapeutic agent for the intended storage condition.

A minimum rate of change in one or more of these criteria (e.g., one or more of appearance, pH, osmolality, protein concentration, CCIT, sterility, visible particulates, particulate matter, SEC, iCIEF, CGE (reduced and non-reduced), Polysorbate 80 content, ADCC activity, and/or CDC activity) can include less than 50% (e.g., less than 45%, less than 40%, less than 35%, less than 30%, less than 25%, less than 20%, less than 15%, less than 10%, less than 5%, less than 4%, less than 3%, less than 2%, or less than 1%) change in one or more of these criteria in a therapeutic agent at the end of an observation period (e.g., after about 1 week, after about 2 weeks, after about 3 weeks, after about 1 month, after about 2 months, after about 3 months, after about 4 months, after about 5 months, after about 6 months, after about 7 months, after about 8 months, after about 9 months, after about 10 months, after about 11 months, after about 12 months, after about 15 months, after about 18 months, after about 24 months, after about 36 months, or more), as compared to that at the start of the observation period.

In some embodiments, a therapeutic agent (e.g., an anti-CD20 antibody) or a pharmaceutical composition comprising the therapeutic agent is stable for up to about 36 months (e.g., up to about 30 months, up to about 24 months, up to about 18 months, up to about 12 months, up to about 11 months, up to about 10 months, up to about 9 months, up to about 8 months, up to about 7 months, up to about 6 months, up to about 5 months, up to about 4 months, up to about 6-36 months, up to about 12-36 months, up to about 18-36 months, up to about 24-36 months, up to about 6-12 months, up to about 9-15 months, up to about 12-18 months, up to about 15-21 months, up to about 18-24 months, up to about 21-27 months, up to about 24-30 months, up to about 27-33 months, up to about 30-36 months, up to about 6-36 months, up to about 12-30 months, or up to about 18-24 months) under a storage condition of 2-8° C. (5±3° C.). For example, a pharmaceutical composition comprising a therapeutic agent (e.g., an anti-CD20 antibody) can be stable for about 6 months under a storage condition of 2-8° C. (5±3° C.).

### Single Use Vial

The therapeutic agents can be present in a single dosage form. For example, the present disclosure provides single use vials comprising therapeutic agents (e.g., anti-CD20 antibody proteins and/or a population thereof) in a single dosage form. In some instances, the single use vials comprise a pharmaceutical formulation that comprises therapeutic agents (e.g., anti-CD20 antibody proteins and/or a population thereof as described herein) in a single dosage form.

The single use vials of the present disclosure comprise pharmaceutical formulations comprising about 75 mg/ml to 500 mg/ml (e.g., about 100 mg/ml to 500 mg/ml; 125 mg/ml to 500 mg/ml; 150 mg/ml to 500 mg/ml; 175 mg/ml to 500 mg/ml; 200 mg/ml to 500 mg/ml; 225 mg/ml to 500 mg/ml; 250 mg/ml to 500 mg/ml; 275 mg/ml to 500 mg/ml; 300 mg/ml to 500 mg/ml; 325 mg/ml to 500 mg/ml; 350 mg/ml to 500 mg/ml; 375 mg/ml to 500 mg/ml; 400 mg/ml to 500 mg/ml; 425 mg/ml to 500 mg/ml; 450 mg/ml to 500 mg/ml; 475 mg/ml to 500 mg/ml; 75 mg/ml to 400 mg/ml; 100 mg/ml to 300 mg/ml; 150 mg/ml to 250 mg/ml; 175 mg/ml to 225 mg/ml; 75 mg/ml to 150 mg/ml; 75 mg/ml to 175 mg/ml; 100 mg/ml to 200 mg/ml; 125 mg/ml to 225 mg/ml; 150 mg/ml to 250 mg/ml; 175 mg/ml to 275 mg/ml; 200 mg/ml to 300 mg/ml; 225 mg/ml to 325 mg/ml; 250 mg/ml to 350 mg/ml; 275 mg/ml to 375 mg/ml; 300 mg/ml to 400 mg/ml; 325 mg/ml to 425 mg/ml; 350 mg/ml to 450 mg/ml; 375 mg/ml to 475 mg/ml; or 400 mg/ml to 500 mg/ml (e.g., about 75 mg/ml; about 100 mg/ml; about 125 mg/ml; about 150 mg/ml; about 175 mg/ml; about 200 mg/ml; about 225 mg/ml; about 250 mg/ml; about 275 mg/ml; about 300 mg/ml; about 325 mg/ml; about 350 mg/ml; about 375 mg/ml; about 400 mg/ml; about 425 mg/ml; about 450 mg/ml; about 475 mg/ml; or about 500 mg/ml)) therapeutic agents (e.g., anti-CD20 antibody proteins and/or a population thereof) in a single dosage form. For example, described herein are single use vials that comprise pharmaceutical formulations comprising about 100 mg/ml to about 300 mg/ml (e.g., about 200 mg/ml) therapeutic agents (e.g., anti-CD20 antibody proteins and/or a population thereof). In some instances, the therapeutic agents (e.g., anti-CD20 antibody proteins and/or a population thereof) is present in the single use vial in a single dosage form.

A single dosage of therapeutic agents (e.g., anti-CD20 antibody proteins and/or a population thereof) can be present in the pharmaceutical formulations in a volume of about 0.5 ml to 5 ml (e.g., 0.5 ml to 4.5 ml; 0.5 ml to 4.0 ml; 0.5 ml to 3.5 ml; 0.5 ml to 3.0 ml; 0.5 ml to 2.5 ml; 0.5 ml to 2.0 ml; 0.5 ml to 1.5 ml; 0.5 ml to 1.0 ml; 1 ml to 5.0 ml, 1 ml to 4.5 ml; 1 ml to 4.0 ml; 1 ml to 3.5 ml; 1 ml to 3.0 ml; 1 ml to 2.5 ml, 1 ml to 2.0 ml; 1 ml to 1.5 ml; 2 ml to 5.0 ml, 2 ml to 4.5 ml; 2 ml to 4.0 ml; 2 ml to 3.5 ml; 2 ml to 3.0 ml; 2 ml to 2.5 ml; 3 ml to 5.0 ml, 3 ml to 4.5 ml; 3 ml to 4.0 ml; 3 ml to 3.5 ml; 4 ml to 5.0 ml, or 4 ml to 4.5 ml (e.g., 0.5 ml; 1 ml; 1.5 ml; 2 ml; 2.5 ml; 3 ml; 3.5 ml; 4 ml; 4.5 ml; or 5 ml)) in single use vials. Single use vials described here can have a capacity of 1 ml to 20 ml (e.g., 1 ml to 15 ml; 1 ml to 10 ml; 1 ml to 5 ml; 5 ml to 20 ml; 5 ml to 15 ml; 5 ml to 10 ml; 10 ml to 20 ml; or 10 ml to 15 ml (e.g., 1 ml; 2 ml; 3 ml; 4 ml; 5 ml; 6 ml; 7 ml; 8 ml; 9 ml; 10 ml; 11 ml; 12 ml; 13 ml; 14 ml; 15 ml; or 20 ml)).

In some instances, the present disclosure provides about 75 mg/ml, 100 mg/ml, 125 mg/ml, 150 mg/ml, 175 mg/ml, 200 mg/ml, 225 mg/ml, 250 mg/ml, 275 mg/ml, 300 mg/ml, 325 mg/ml, 350 mg/ml, 375 mg/ml, 400 mg/ml, 425 mg/ml, 450 mg/ml, 475 mg/ml, or 500 mg/ml therapeutic agents (e.g., anti-CD20 antibody proteins (or a population thereof) in a pharmaceutical formulation in a volume of 1 ml, 2 ml, 3 ml, 4 ml, 5 ml, 6 ml, 7 ml, 8 ml, 9 ml, or 10 ml in a single use vial (e.g., a single use vial with a capacity of 1 ml, 2 ml, 3 ml, 4 ml, 5 ml, 6 ml, 7 ml, 8 ml, 9 ml, or 10 ml). In some instances, the present disclosure provides about 100 mg/mL to about 300 mg/mL (e.g., about 200 mg/mL) therapeutic agents (e.g., anti-CD20 antibody proteins or a population thereof) in a pharmaceutical formulation in a volume of 1 ml, 2 ml, 3 ml, 4 ml, 5 ml, 6 ml, 7 ml, 8 ml, 9 ml, or 10 ml in a single use vial. For example, a single use vial of the present disclosure can comprise a pharmaceutical formulation comprising about 200 mg/ml of therapeutic agents (e.g., anti-CD20 antibody proteins or a population thereof) in a total volume of 2 ml.

In some instances, the pharmaceutical formulation in a single use vial comprises about 200 mg/ml of therapeutic agents (e.g., anti-CD20 antibody proteins or a population thereof) in a total volume of 2 ml, and one, two, three, or more of the following: arginine, glutamate, and polysorbate 80. For example, a single use vial of the present disclosure can comprise a pharmaceutical formulation comprising about 200 mg/ml of anti-CD20 antibody proteins (or a population thereof) in a total volume of 2 ml, and one or more of the following: 114 mM to 192 mM (e.g., 166±10% mM) arginine (e.g., L-arginine), 184 mM to 242 mM (e.g., 224±10% mM) glutamate (e.g., L-glutamic acid), and 0.03% to 0.07% (e.g., 0.045% to 0.055% or about 0.05%) polysorbate 80. In some instances, the pharmaceutical formulation in the single use vials further comprises sodium hydroxide, e.g., for adjusting the pH of the pharmaceutical formulation to a pH of about 4.95 to about 6.05 (e.g., about 5.5). In some instances, the pharmaceutical formulation in a single use vial comprises water (e.g., to adjust the volume of the pharmaceutical formulation to about 2 ml).

The pharmaceutical formulation in a single use vial can comprises about 400 mg of therapeutic agents (e.g., anti-CD20 antibody proteins or a population thereof) in a total volume of about 2 ml, and one or more of the following: arginine (e.g., L-arginine), glutamate (e.g., L-glutamic acid), and polysorbate 80. For example, a single use vial of the present disclosure can comprise a pharmaceutical formulation comprising about 400 mg of therapeutic agents (e.g., the anti-CD20 antibody proteins or a population thereof) in a total volume of 2 ml, and one or more of the following: 39.7 mg to 66.9 mg (e.g., 57.8±10% mg) arginine (e.g., L-arginine), 54.1 mg to 71.2 mg (e.g., 65.9±10% mg) glutamate (e.g., L-glutamic acid), and 1.1±10% mg polysorbate 80. The pharmaceutical formulation in a single use vial can further comprise sodium hydroxide (e.g., for adjusting the pH of the pharmaceutical formulation to a pH of about 5.5). In some instances, the pharmaceutical formulation in a single use vial comprises water (e.g., to adjust the volume of the pharmaceutical formulation to about 2 ml).

### Pre-Filled Syringe

The therapeutic agents (e.g., antibody proteins or a population thereof) can be present in a single dosage form. For example, the present disclosure provides pre-filled syringes comprising therapeutic agents (e.g., anti-CD20 antibody proteins and/or a population thereof) in a single dosage form. The pre-filled syringes of the present disclosure can be autoinjectors (e.g., autoinjector pens). For example, described herein are autoinjectors comprising therapeutic agents (e.g., anti-CD20 antibody proteins and/or a population thereof) in a single dosage form.

The pre-filled syringes of the present disclosure can comprise a staked-needle assembly (SNA) assembled into a platform autoinjector. The SNA can comprise a glass syringe with a staked needle. The syringe can be a 1 mL to 5 mL, or 1.25 mL to 3.25 mL (e.g., 1.5 mL to 4.5 mL, 2.0 mL to 4.0 mL, 1.5 mL to 5.0 mL, 2.0 mL to 5.0 mL, 2.5 mL to 5.0 mL, 3.0 mL to 5.0 mL, 3.5 mL to 5.0 mL, 4.0 mL to 5.0 mL, 4.5 mL to 5.0 mL, 1.5 mL to 3.0 mL, 1.75 mL to 2.75 mL, 2.0 mL to 2.5 mL, 1.0 mL, 1.25 mL, 1.5 mL, 1.75 mL, 2.0 mL, 2.25 mL, 2.5 mL, 2.75 mL, 3.0 mL, 3.25 mL, 3.5 mL, 3.75 mL, 4.0 mL, 4.25 mL, 4.5 mL, 4.75 mL, or 5.0 mL) staked-needle glass syringe. Thus, the SNA can comprise a 2.25 mL or 3.0 mL staked-needle glass syringe. The needle can be a thin wall needle, such as a 25-gauge to 30-gauge (e.g., 25-gauge to 27-gauge, 26-gauge to 28-gauge, 27-gauge to 29-gauge, 28-gauge to 30-gauge, 25-gauge, 26-gauge, 27-gauge, 28-gauge, 29-gauge, or 30-gauge) needle. For example, the needle can be a 27-gauge to 29-gauge (e.g., 27-gauge) thin wall needle. The needle can be about 10 mm to about 20 mm (e.g., about 11 mm to about 19 mm, about 12 mm to about 18 mm, about 13 mm to about 17 mm, about 13 mm to about 16 mm, about 14 mm to about 16 mm) long. For example, the needle can be 13 mm to 16 mm long. Once filled with the pharmaceutical formulation described herein, the syringe can be closed with a rubber plunger stopper to form the SNA.

The SNAs can be assembled into a platform autoinjector. Platform autoinjectors are known in the art. See, e.g., U.S. Pat. Nos. 9,199,038, 9,220,841, 9,662,452, 8,821,451, 9,713,677, 10,092,704, 10,420,898, 10,130,774, 10,933,197, 11,291,775, 12,156,993, 11,771,839, 11,819,669, 11,826,554, 11,819,671, 12,023,472, 11,883,637, 12,161,849, and 11,458,254, the contents of which are incorporated herein by reference. Platform autoinjectors for use in the compositions and methods of the present disclosure can comprise a front sub-assembly and a rear sub-assembly. The front sub-assembly can comprise the SNA, a cap with a gripper to remove the rigid needle shield, and a clear viewing window. The rear sub-assembly or “power pack” can provide the spring force mechanism, which depresses the plunger to expel the content of the syringe (e.g., the pharmaceutical formulation of the present disclosure) once the autoinjector is activated. Autoinjectors for use in the present methods can deliver the content (e.g., the pharmaceutical formulation of the present disclosure) over 5 to 20 seconds, 7 to 17 seconds, or 10 to 15 seconds (e.g., about 5 seconds, 7 seconds, 10 seconds, 12 seconds, 15 seconds, 17 seconds, or 20 seconds; such as, about 10 seconds). For example, autoinjectors for use in the present methods can deliver about 400 mg of the therapeutic agents (e.g., anti-CD20 antibody proteins or a population thereof) in a total volume of about 2 ml, over 10 seconds.

The pre-filled syringes (e.g., autoinjectors) of the present disclosure can comprise a pharmaceutical formulation that comprises therapeutic agents (e.g., anti-CD20 antibody proteins and/or a population thereof) in a single dosage form. Thus, the pre-filled syringes (e.g., autoinjectors) described herein can comprise pharmaceutical formulations comprising about 75 mg/ml to 500 mg/ml (e.g., about 100 mg/ml to 500 mg/ml; 125 mg/ml to 500 mg/ml; 150 mg/ml to 500 mg/ml; 175 mg/ml to 500 mg/ml; 200 mg/ml to 500 mg/ml; 225 mg/ml to 500 mg/ml; 250 mg/ml to 500 mg/ml; 275 mg/ml to 500 mg/ml; 300 mg/ml to 500 mg/ml; 325 mg/ml to 500 mg/ml; 350 mg/ml to 500 mg/ml; 375 mg/ml to 500 mg/ml; 400 mg/ml to 500 mg/ml; 425 mg/ml to 500 mg/ml; 450 mg/ml to 500 mg/ml; 475 mg/ml to 500 mg/ml; 75 mg/ml to 400 mg/ml; 100 mg/ml to 300 mg/ml; 150 mg/ml to 250 mg/ml; 175 mg/ml to 225 mg/ml; 75 mg/ml to 150 mg/ml; 75 mg/ml to 175 mg/ml; 100 mg/ml to 200 mg/ml; 125 mg/ml to 225 mg/ml; 150 mg/ml to 250 mg/ml; 175 mg/ml to 275 mg/ml; 200 mg/ml to 300 mg/ml; 225 mg/ml to 325 mg/ml; 250 mg/ml to 350 mg/ml; 275 mg/ml to 375 mg/ml; 300 mg/ml to 400 mg/ml; 325 mg/ml to 425 mg/ml; 350 mg/ml to 450 mg/ml; 375 mg/ml to 475 mg/ml; or 400 mg/ml to 500 mg/ml (e.g., about 75 mg/ml; about 100 mg/ml; about 125 mg/ml; about 150 mg/ml; about 175 mg/ml; about 200 mg/ml; about 225 mg/ml; about 250 mg/ml; about 275 mg/ml; about 300 mg/ml; about 325 mg/ml; about 350 mg/ml; about 375 mg/ml; about 400 mg/ml; about 425 mg/ml; about 450 mg/ml; about 475 mg/ml; or about 500 mg/ml)) therapeutic agents (e.g., anti-CD20 antibody proteins and/or a population thereof) in a single dosage form. For example, described herein are pre-filled syringes (e.g., autoinjectors) that comprise pharmaceutical formulations comprising about 100 mg/ml to about 300 mg/ml (e.g., about 150 mg/ml to 250 mg/ml or about 200 mg/ml) therapeutic agents (e.g., anti-CD20 antibody proteins and/or a population thereof). In some instances, the therapeutic agents (e.g., anti-CD20 antibody proteins and/or a population thereof) is present in the pre-filled syringes (e.g., autoinjectors) in a single dosage form.

Single dosage of therapeutic agents (e.g., anti-CD20 antibody proteins and/or a population thereof as described herein) can be present in the pre-filled syringes (e.g., autoinjectors) s in a volume of about 0.5 ml to 5 ml (e.g., 0.5 ml to 4.5 ml; 0.5 ml to 4.0 ml; 0.5 ml to 3.5 ml; 0.5 ml to 3.0 ml; 0.5 ml to 2.5 ml; 0.5 ml to 2.0 ml; 0.5 ml to 1.5 ml; 0.5 ml to 1.0 ml; 1 ml to 5.0 ml, 1 ml to 4.5 ml; 1 ml to 4.0 ml; 1 ml to 3.5 ml; 1 ml to 3.0 ml; 1 ml to 2.5 ml, 1 ml to 2.0 ml; 1 ml to 1.5 ml; 2 ml to 5.0 ml, 2 ml to 4.5 ml; 2 ml to 4.0 ml; 2 ml to 3.5 ml; 2 ml to 3.0 ml; 2 ml to 2.5 ml; 3 ml to 5.0 ml, 3 ml to 4.5 ml; 3 ml to 4.0 ml; 3 ml to 3.5 ml; 4 ml to 5.0 ml, or 4 ml to 4.5 ml (e.g., 0.5 ml; 1 ml; 1.5 ml; 2 ml; 2.5 ml; 3 ml; 3.5 ml; 4 ml; 4.5 ml; or 5 ml)) in pre-filled syringes (e.g., autoinjectors). In some instances, pre-filled syringes (e.g., autoinjectors) described here have a capacity of 1 ml to 5 ml (e.g., 1 ml; 2 ml; 3 ml; 4 ml; or 5 ml). For example, pre-filled syringes (e.g., autoinjectors) described here can have a capacity of about 3 ml.

In some instances, the present disclosure provides 75 mg/ml, 100 mg/ml, 125 mg/ml, 150 mg/ml, 175 mg/ml, 200 mg/ml, 225 mg/ml, 250 mg/ml, 275 mg/ml, 300 mg/ml, 325 mg/ml, 350 mg/ml, 375 mg/ml, 400 mg/ml, 425 mg/ml, 450 mg/ml, 475 mg/ml, or 500 mg/ml therapeutic agents (e.g., anti-CD20 antibody proteins and/or a population thereof as described herein) in a pharmaceutical formulation in a volume of 1 ml, 2 ml, 3 ml, 4 ml, or 5 ml in a pre-filled syringe (e.g., a pre-filled syringe with a capacity of 1 ml, 2 ml, 3 ml, 4 ml, or 5 ml). In some instances, the present disclosure provides 200 mg/ml therapeutic agents (e.g., anti-CD20 antibody proteins and/or a population thereof) in a pharmaceutical formulation in a volume of 1 ml, 2 ml, 3 ml, 4 ml, 5 ml, 6 ml, 7 ml, 8 ml, 9 ml, or 10 ml in a pre-filled syringe (e.g., a pre-filled syringe with a capacity of 5 ml or 10 ml). For example, a pre-filled syringe (e.g., autoinjector) of the present disclosure can comprise a pharmaceutical formulation comprising about 200 mg/ml of the therapeutic agents (e.g., anti-CD20 antibody proteins or a population thereof) in a total volume of 2 ml.

The pharmaceutical formulation in a pre-filled syringe (e.g., autoinjector) can comprises about 200 mg/ml of therapeutic agents (e.g., anti-CD20 antibody proteins or a population thereof as described herein) in a total volume of 2 ml, and one or more of the following: L-arginine, L-glutamic acid, and polysorbate 80. For example, a pre-filled syringe (e.g., autoinjector) of the present disclosure can comprise a pharmaceutical formulation comprising about 200 mg/ml of therapeutic agents (e.g., anti-CD20 antibody proteins or a population thereof as described herein) in a total volume of 2 ml, and one or more of the following: 114 mM to 192 mM (e.g., 166±10% mM) arginine (e.g., L-arginine), 184 mM to 242 mM (e.g., 224±10% mM) glutamate (e.g., L-glutamic acid), and 0.03% to 0.07% (e.g., 0.045% to 0.055% or about 0.05%) polysorbate 80. The pharmaceutical formulation in a pre-filled syringe (e.g., autoinjector) can further comprise sodium hydroxide (e.g., for adjusting the pH of the pharmaceutical formulation to a pH of about 5.5). In some instances, the pharmaceutical formulation in a pre-filled syringe (e.g., autoinjector) comprises water (e.g., to adjust the volume of the pharmaceutical formulation to about 2 ml).

The pharmaceutical formulation in a pre-filled syringe (e.g., autoinjector) can comprise about 400 mg of the therapeutic agents (e.g., anti-CD20 antibody proteins or a population thereof as described herein) in a total volume of about 2 ml, and one or more of the following: arginine (e.g., L-arginine), glutamate (e.g., L-glutamic acid), and polysorbate 80. For example, a pre-filled syringe (e.g., autoinjector) of the present disclosure can comprise a pharmaceutical formulation comprising about 400 mg of therapeutic agents (e.g., the anti-CD20 antibody proteins or a population thereof as described herein) in a total volume of 2 ml, and one or more of the following: about 39.7 mg to about 66.9 mg (e.g., about 57.8±10% mg) L-arginine, about 54.1 mg to about 71.2 mg (e.g., about 65.9±10% mg) L-glutamic acid, and about 1.1±10% mg polysorbate 80. The pharmaceutical formulation in a pre-filled syringe (e.g., autoinjector) can further comprise sodium hydroxide (e.g., for adjusting the pH of the pharmaceutical formulation to a pH of about 5.5). In some instances, the pharmaceutical formulation in a pre-filled syringe (e.g., autoinjector) comprises water (e.g., to adjust the volume of the pharmaceutical formulation to about 2 ml).

### Methods of Treating and Preventing Medical Conditions

Also provided herein are methods for treating and/or preventing one or more medical conditions in a subject (e.g., a human subject, such as, a patient) in need thereof, by administering to the subject the pharmaceutical formulations (e.g., comprising anti-CD20 antibody proteins) of the present disclosure.

### Anti-CD20 Antibody Used in the Treatment Methods

In some embodiments, the anti-CD20 antibody protein used in the present methods is the anti-CD20 antibody protein as described herein, e.g., a population of anti-CD20 antibody proteins expressed from one or more nucleic acid sequences encoding a heavy chain comprising the amino acid sequence of SEQ ID NO:1 and a light chain comprising the amino acid sequence of SEQ ID NO:2, wherein the population of anti-CD20 antibody proteins has an N-glycan profile comprising 10 to 20% galactosylated glycans and about 20 to 40% fucosylated glycans. In some embodiments, the anti-CD20 antibody protein used in the present methods is the anti-CD20 antibody protein as described herein, e.g., anti-CD20 antibody protein in a population comprising a heavy chain comprising the amino acid sequence of SEQ ID NO: 1 and a light chain comprising the amino acid sequence of SEQ ID NO:2, wherein the population of anti-CD20 antibody proteins has an N-glycan profile comprising 10 to 20% galactosylated glycans and about 20 to 40% fucosylated glycans. In some embodiments, the anti-CD20 antibody protein used in the present methods is the anti-CD20 antibody protein as described herein, e.g., anti-CD20 antibody protein in a population, wherein the population of anti-CD20 antibody proteins has an N-glycan profile comprising 10 to 20% galactosylated glycans and about 20 to 40% fucosylated glycans, and wherein the anti-CD20 antibody proteins in the population comprise: a VH comprising a VH CDR1 comprising the amino acid sequence of SEQ ID NO:3, a VH CDR2 comprising the amino acid sequence of SEQ ID NO:4, and a VH CDR3 comprising the amino acid sequence of SEQ ID NO: 5; and/or a VL comprising a VL CDR1 comprising the amino acid sequence of SEQ ID NO: 8, a VL CDR2 comprising the amino acid sequence of SEQ ID NO:9, and a VL CDR3 comprising the amino acid sequence of SEQ ID NO: 10.

In some embodiments, the method comprises administering to the subject a pharmaceutical formulation described herein. For example, a pharmaceutical formulation of the present disclosure is administered to the subject subcutaneously (e.g., by subcutaneous injection).

A pharmaceutical formulation comprising about 150 mg to about 1000 mg (e.g., about 200 mg to about 1000 mg, about 250 mg to about 1000 mg, about 300 mg to about 1000 mg, about 350 mg to about 1000 mg, about 400 mg to about 1000 mg, about 450 mg to about 1000 mg, about 500 mg to about 1000 mg, about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, about 900 mg to about 1000 mg, about 950 mg to about 1000 mg, about 200 mg to about 900 mg, about 300 mg to about 800 mg, about 400 mg to about 700 mg, about 500 mg to about 600 mg, about 150 mg to about 250 mg, about 200 mg to about 300 mg, about 250 mg to about 350 mg, about 300 mg to about 400 mg, about 350 mg to about 450 mg, about 400 mg to about 500 mg, about 450 mg to about 550 mg, about 500 mg to about 600 mg, about 550 mg to about 650 mg, about 600 mg to about 700 mg, about 650 mg to about 750 mg, about 700 mg to about 800 mg, about 750 mg to about 850 mg, about 800 mg to about 900 mg, about 850 mg to about 950 mg, or about 900 mg to about 1000 mg (e.g., about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg)) of the anti-CD20 antibody protein can be administered to a subject by subcutaneous injection. For example, a pharmaceutical formulation comprising about 300 mg of the anti-CD20 antibody protein can be administered to a subject by subcutaneous injection. For administering about 300 mg of the anti-CD20 antibody protein composition to a subject, about 1.5 ml of a pharmaceutical formulation comprising the anti-CD20 antibody protein at a concentration of about 200 mg/ml can be administered to the subject by subcutaneous injection. Also, a pharmaceutical formulation comprising about 400 mg of the anti-CD20 antibody protein can be administered to a subject by subcutaneous injection. For administering about 400 mg of the anti-CD20 antibody protein composition to a subject, about 2.0 ml of a pharmaceutical formulation comprising the anti-CD20 antibody protein at a concentration of about 200 mg/ml can be administered to the subject by subcutaneous injection. Also, a pharmaceutical formulation comprising about 800 mg of the anti-CD20 antibody protein can be administered to a subject by subcutaneous injection. For administering about 800 mg of the anti-CD20 antibody protein composition to a subject, two separate 400 mg doses can be consecutively administered to the subject by subcutaneous injections. For example, a 800 mg dose of the anti-CD20 antibody protein composition can be administered to a subject over two consecutive subcutaneous injections, each administering about 2.0 ml of a pharmaceutical formulation comprising the anti-CD20 antibody protein at a concentration of about 200 mg/ml. The subcutaneous injection can be administered to the subject by a healthcare professional (e.g., a physician, a nurse practitioner, a nurse, a physician assistant, a nursing assistant, and/or a medical assistant). In some instances, the subcutaneous injection is self-administered by the subject. Alternatively, the subcutaneous injection is not self-administered by the subject.

A pharmaceutical formulation comprising about 75 mg/ml to about 500 mg/ml (e.g., about 100 mg/ml to 500 mg/ml, 125 mg/ml to 500 mg/ml, 150 mg/ml to 500 mg/ml, 175 mg/ml to 500 mg/ml, 200 mg/ml to 500 mg/ml, 225 mg/ml to 500 mg/ml, 250 mg/ml to 500 mg/ml, 275 mg/ml to 500 mg/ml, 300 mg/ml to 500 mg/ml, 325 mg/ml to 500 mg/ml, 350 mg/ml to 500 mg/ml, 375 mg/ml to 500 mg/ml, 400 mg/ml to 500 mg/ml, 425 mg/ml to 500 mg/ml, 450 mg/ml to 500 mg/ml, 475 mg/ml to 500 mg/ml, 75 mg/ml to 400 mg/ml, 100 mg/ml to 300 mg/ml, 150 mg/ml to 250 mg/ml, 175 mg/ml to 225 mg/ml, 75 mg/ml to 150 mg/ml, 75 mg/ml to 175 mg/ml, 100 mg/ml to 200 mg/ml, 125 mg/ml to 225 mg/ml, 150 mg/ml to 250 mg/ml, 175 mg/ml to 275 mg/ml, 200 mg/ml to 300 mg/ml, 225 mg/ml to 325 mg/ml, 250 mg/ml to 350 mg/ml, 275 mg/ml to 375 mg/ml, 300 mg/ml to 400 mg/ml, 325 mg/ml to 425 mg/ml, 350 mg/ml to 450 mg/ml, 375 mg/ml to 475 mg/ml, or 400 mg/ml to 500 mg/ml (e.g., about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, about 300 mg/ml, about 325 mg/ml, about 350 mg/ml, about 375 mg/ml, about 400 mg/ml, about 425 mg/ml, about 450 mg/ml, about 475 mg/ml, or about 500 mg/ml)) of the anti-CD20 antibody protein can be administered to a subject by subcutaneous injection. For example, a pharmaceutical formulation comprising about 200 mg/ml of the anti-CD20 antibody protein can be administered to a subject by subcutaneous injection. A pharmaceutical formulation comprising about 200 mg/ml of the anti-CD20 antibody protein in a total volume of about 2 ml can be administered to a subject by subcutaneous injection. The subcutaneous injection can be administered to the subject by a healthcare professional (e.g., a physician, a nurse practitioner, a nurse, a physician assistant, a nursing assistant, and/or a medical assistant). In some instances, the subcutaneous injection is self-administered by the subject. Alternatively, the subcutaneous injection is not self-administered by the subject.

### Subcutaneous Administration

The pharmaceutical formulations comprising anti-CD20 antibody proteins can be administered at one or more sites (or locations) in the body of a subject. Sites (or locations) for administration of subcutaneous injections in the body of a subject can include one or more of the following: the outer lateral aspect of the upper arm, the abdomen (e.g., from below the costal margin to the iliac crest and more than two inches from the umbilicus), the anterior upper thighs, the upper back, and the upper ventral gluteal area. For example, an anti-CD20 antibody protein composition as described herein can be administered to a subject in the lower abdomen (e.g., into the subcutaneous tissue of lower abdomen), e.g., about 2 inches or more (e.g., about 2 inches, about 2.5 inches, about 3 inches, about 3.5 inches, about 4 inches, about 4.5 inches, about 5 inches, about 5.5 inches, about 6 inches, or more) from the navel or umbilicus. In some instances, the anti-CD20 antibody protein composition is not administered in an area where there is bruising, redness, tenderness, hardness, or near a mole, scar or stretch marks.

A single dose of an anti-CD20 antibody protein composition described here can be administered by a single subcutaneous injection. Alternatively, a single dose of an anti-CD20 antibody protein composition described here can be administered by multiple (i.e., more than one) subcutaneous injections (e.g., can be split over multiple administrations). For example, a single dose of an anti-CD20 antibody protein composition that is present in a large volume (e.g., more than 1.5 ml or 2 ml (e.g., 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, or more)) can be administered by multiple (i.e., more than one) subcutaneous injections, wherein each subcutaneous injection administers about 1-2 ml of a pharmaceutical formulation comprising the anti-CD20 antibody protein at a concentration of about 200 mg/ml. Thus, about 300 mg of the anti-CD20 antibody protein composition can be administered to a subject as one subcutaneous injection (e.g., a single subcutaneous injection administering 1.5 ml of a pharmaceutical formulation comprising the anti-CD20 antibody protein at a concentration of about 200 mg/ml). Alternatively, about 300 mg of the anti-CD20 antibody protein composition can be administered to a subject as multiple (e.g., more than one) subcutaneous injections. Also, about 400 mg of the anti-CD20 antibody protein composition can be administered to a subject as one subcutaneous injection (e.g., a single subcutaneous injection administering 2.0 ml of a pharmaceutical formulation comprising the anti-CD20 antibody protein at a concentration of about 200 mg/ml). Alternatively, about 400 mg of the anti-CD20 antibody protein composition can be administered to a subject as multiple (e.g., more than one) subcutaneous injections. Also, about 800 mg of the anti-CD20 antibody protein composition can be administered to a subject as one subcutaneous injection. Alternatively, about 800 mg of the anti-CD20 antibody protein composition can be administered to a subject as multiple (e.g., more than one) subcutaneous injections. For example, about 800 mg of the anti-CD20 antibody protein composition can be administered to a subject as two consecutive subcutaneous injections, each administering 2.0 ml of a pharmaceutical formulation comprising the anti-CD20 antibody protein at a concentration of about 200 mg/ml. When a single dose of an anti-CD20 antibody protein composition is administered by multiple (i.e., more than one) subcutaneous injections, the injection can be administered at multiple sites. The sites for administering the multiple subcutaneous injections can be spaced about 2 inches or more (e.g., about 2 inches, about 2.5 inches, about 3 inches, about 3.5 inches, about 4 inches, about 4.5 inches, about 5 inches, about 5.5 inches, about 6 inches, or more) from each other. For example, about 800 mg of the anti-CD20 antibody protein composition can be administered to a subject as two consecutive subcutaneous injections (each administering 2.0 ml of a pharmaceutical formulation comprising the anti-CD20 antibody protein at a concentration of about 200 mg/ml), wherein the two subcutaneous injections are administered at two different sites in the lower abdomen, spaced at least 2 inches (e.g., about 2 inches, about 2.5 inches, about 3 inches, about 3.5 inches, about 4 inches, about 4.5 inches, about 5 inches, about 5.5 inches, about 6 inches, or more) apart and at least 2 inches (e.g., about 2 inches, about 2.5 inches, about 3 inches, about 3.5 inches, about 4 inches, about 4.5 inches, about 5 inches, about 5.5 inches, about 6 inches, or more) from the navel or umbilicus.

The anti-CD20 antibody protein composition described herein can be administered to a subject subcutaneously (e.g., by subcutaneous injection). The method can include the following: a first dose of the anti-CD20 antibody protein composition administered to the subject by subcutaneous injection at day 1; a second dose of the anti-CD20 antibody protein composition administered to the subject by subcutaneous injection at about 2 weeks after the first dose (e.g., at day 15); a third dose of the anti-CD20 antibody protein composition administered to the subject by subcutaneous injection at about 12 weeks (e.g., about 3 months) after the first dose; and one or more subsequent doses of the anti-CD20 antibody protein composition administered to the subject by subcutaneous injection at about 12 weeks (e.g., about 3 months) after the prior dose. The method can alternatively include the following: a first dose of the anti-CD20 antibody protein composition administered to the subject by subcutaneous injection at day 1; a second dose of the anti-CD20 antibody protein composition administered to the subject by subcutaneous injection at about 2 weeks after the first dose (e.g., at day 15); a third dose of the anti-CD20 antibody protein composition administered to the subject by subcutaneous injection at about 8 weeks (e.g., about 2 months) after the first dose; and one or more subsequent doses of the anti-CD20 antibody protein composition administered to the subject by subcutaneous injection at about 8 weeks (e.g., about 2 months) after the prior dose. In some instances, the anti-CD20 antibody protein composition is administered to the subject by subcutaneous injection at Day 1, Day 15, Week 12, Week 24, Week 36, Week 48, Week 60, Week 72, Week 84, Week 96, and every 12 weeks (or about 3 months) thereafter. In some instances, the subject is not administered the subcutaneous anti-CD20 antibody protein composition at Day 15. For example, the anti-CD20 antibody protein composition can be administered to the subject by subcutaneous injection at Day 1, Week 12, Week 24, Week 36, Week 48, Week 60, Week 72, Week 84, Week 96, and every 12 weeks (or about 3 months) thereafter. In some instances, the subcutaneous anti-CD20 antibody protein composition is administered subcutaneously to the subject at Day 1, Day 15, Week 8, Week 16, Week 24, Week 32, Week 40, Week 48, Week 56, Week 64, Week 72, Week 80, Week 88, Week 96, and every 8 weeks (or about 2 months) thereafter. In some instances, the subject is not administered the subcutaneous anti-CD20 antibody protein composition at Day 15. For example, the subcutaneous anti-CD20 antibody protein composition can be administered subcutaneously to the subject at Day 1, Week 8, Week 16, Week 24, Week 32, Week 40, Week 48, Week 56, Week 64, Week 72, Week 80, Week 88, Week 96, and every 8 weeks (or about 2 months) thereafter.

The first dose can comprise the population of anti-CD20 antibody proteins in an amount that is bioequivalent to about 150 mg of the population of anti-CD20 antibody proteins administered intravenously. The second dose can comprise the population of anti-CD20 antibody proteins in an amount that is bioequivalent to about 225 mg of the population of anti-CD20 antibody proteins administered intravenously. The third dose can comprise the population of anti-CD20 antibody proteins in an amount that is bioequivalent to about 225 mg of the population of anti-CD20 antibody proteins administered intravenously. The one or more subsequent doses can comprise the population of anti-CD20 antibody proteins in an amount that is bioequivalent to about 225 mg of the population of anti-CD20 antibody proteins administered intravenously. The bioequivalent of the population of anti-CD20 antibody proteins (e.g., bioequivalent of the 150 mg and/or 225 mg of the population of anti-CD20 antibody proteins) administered intravenously can be determined based on the PK/PD and tolerability data, as described in Example 16 of the present disclosure. In some instances, one or more of the first dose, the second dose, the third dose, and the one or more subsequent doses can each comprise about 250 mg to about 500 mg (e.g., about 265 mg to about 450 mg; optionally, about 300 mg or about 400 mg) of the population of anti-CD20 antibody proteins. For example, the first dose can comprise about 250 mg to about 500 mg (e.g., about 265 mg to about 450 mg; optionally, about 300 mg) of the population of anti-CD20 antibody proteins; the second dose can comprise about 250 mg to about 500 mg (e.g., about 265 mg to about 450 mg; optionally, about 400 mg) of the population of anti-CD20 antibody proteins; the third dose can comprise about 250 mg to about 500 mg (e.g., about 265 mg to about 450 mg; optionally, about 400 mg) of the population of anti-CD20 antibody proteins; and/or the one or more subsequent doses can each comprise about 250 mg to about 500 mg (e.g., about 265 mg to about 450 mg; optionally, about 400 mg) of the population of anti-CD20 antibody proteins. Preferably, the first dose can comprise about 300 mg of the population of anti-CD20 antibody proteins; the second dose can comprise about 400 mg of the population of anti-CD20 antibody proteins; the third dose can comprise about 400 mg of the population of anti-CD20 antibody proteins; and/or the one or more subsequent doses can each comprise about 400 mg of the population of anti-CD20 antibody proteins.

In some instances, one or more of the first dose, the second dose, the third dose, and the one or more subsequent doses can each comprise about 250 mg to about 500 mg (e.g., about 265 mg to about 450 mg; optionally, about 400 mg) of the population of anti-CD20 antibody proteins. For example, the first dose can comprise about 250 mg to about 500 mg (e.g., about 265 mg to about 450 mg; optionally, about 400 mg) of the population of anti-CD20 antibody proteins; the second dose can comprise about 250 mg to about 500 mg (e.g., about 265 mg to about 450 mg; optionally, about 400 mg) of the population of anti-CD20 antibody proteins; the third dose can comprise about 250 mg to about 500 mg (e.g., about 265 mg to about 450 mg; optionally, about 400 mg) of the population of anti-CD20 antibody proteins; and/or the one or more subsequent doses can each comprise about 250 mg to about 500 mg (e.g., about 265 mg to about 450 mg; optionally, about 400 mg) of the population of anti-CD20 antibody proteins. Preferably, the first dose can comprise about 400 mg of the population of anti-CD20 antibody proteins; the second dose can comprise about 400 mg of the population of anti-CD20 antibody proteins; the third dose can comprise about 400 mg of the population of anti-CD20 antibody proteins; and/or the one or more subsequent doses can each comprise about 400 mg of the population of anti-CD20 antibody proteins.

In some instances, one or more of the first dose, the second dose, the third dose, and the one or more subsequent doses can each comprise about 250 mg to about 500 mg (e.g., about 265 mg to about 450 mg; optionally, about 400 mg) of the population of anti-CD20 antibody proteins. For example, the first dose can comprise about 250 mg to about 500 mg (e.g., about 265 mg to about 450 mg; optionally, about 400 mg) of the population of anti-CD20 antibody proteins; the second dose can comprise about 250 mg to about 500 mg (e.g., about 265 mg to about 450 mg; optionally, about 400 mg) of the population of anti-CD20 antibody proteins; the third dose can comprise about 250 mg to about 500 mg (e.g., about 265 mg to about 450 mg; optionally, about 400 mg) of the population of anti-CD20 antibody proteins; and/or the one or more subsequent doses can each comprise about 250 mg to about 500 mg (e.g., about 265 mg to about 450 mg; optionally, about 400 mg) of the population of anti-CD20 antibody proteins. Preferably, the first dose can comprise about 400 mg of the population of anti-CD20 antibody proteins; the second dose can comprise about 400 mg of the population of anti-CD20 antibody proteins; the third dose can comprise about 400 mg of the population of anti-CD20 antibody proteins; and/or the one or more subsequent doses can each comprise about 400 mg of the population of anti-CD20 antibody proteins.

In some instances, the subject is not administered a second dose of the subcutaneous anti-CD20 antibody protein composition. Thus, the first dose can comprise about 250 mg to about 500 mg (e.g., about 265 mg to about 450 mg; optionally, about 400 mg) of the population of anti-CD20 antibody proteins; the second dose is not administered; the third dose can comprise about 250 mg to about 500 mg (e.g., about 265 mg to about 450 mg; optionally, about 400 mg) of the population of anti-CD20 antibody proteins; and/or the one or more subsequent doses can each comprise about 250 mg to about 500 mg (e.g., about 265 mg to about 450 mg; optionally, about 400 mg) of the population of anti-CD20 antibody proteins. Preferably, the first dose can comprise about 400 mg of the population of anti-CD20 antibody proteins; the second dose is not administered; the third dose can comprise about 400 mg of the population of anti-CD20 antibody proteins; and/or the one or more subsequent doses can each comprise about 400 mg of the population of anti-CD20 antibody proteins. Also, the first dose can comprise about 600 mg to about 1000 mg (e.g., about 700 mg to about 900 mg; optionally, about 800 mg) of the population of anti-CD20 antibody proteins; the second dose is not administered; the third dose can comprise about 250 mg to about 500 mg (e.g., about 265 mg to about 450 mg; optionally, about 400 mg) of the population of anti-CD20 antibody proteins; and/or the one or more subsequent doses can each comprise about 250 mg to about 500 mg (e.g., about 265 mg to about 450 mg; optionally, about 400 mg) of the population of anti-CD20 antibody proteins. Preferably, the first dose can comprise about 400 mg of the population of anti-CD20 antibody proteins; the second dose is not administered; the third dose can comprise about 400 mg of the population of anti-CD20 antibody proteins; and/or the one or more subsequent doses can each comprise about 400 mg of the population of anti-CD20 antibody proteins.

### Autoimmune Diseases

Provided herein are methods of treating an autoimmune disease in a subject in need thereof, by administering to the subject an effective amount of an anti-CD20 antibody protein composition of the present disclosure.

Autoimmune diseases cover diseases that occur when the body's immune system attacks its own normal tissues, organs or other in vivo components due to immune system abnormalities whose cause cannot be found. These autoimmune diseases are systemic diseases that can occur in almost all parts of the body, including the nervous system, the gastrointestinal system, the endocrine system, the skin, the skeletal system, and the vascular tissue. It is known that autoimmune diseases affect about 5-8% of the world population, but the reported prevalence of autoimmune diseases is lower than the actual level due to limitations in the understanding of autoimmune diseases and a method for diagnosing these diseases.

In some instances, the autoimmune disease is multiple sclerosis (MS). The MS can be a relapsing form of MS (RMS). For example, provided herein is a method of treating an RMS in a subject in need thereof, by administering to the subject an effective amount of an anti-CD20 antibody protein composition of the present disclosure. The RMS can be selected from a clinically isolated syndrome (“CIS”), relapsing-remitting (“RRMS”), or active secondary progressive disease (“SPMS”). In some instances, the RMS is a CIS. In some instances, the RMS is RRMS. In some instances, the RMS is SPMS. The subject can be diagnosed with RMS in accordance to McDonald Criteria (Thompson et al., The Lancet Neurology, 17(2):162-173 (2017)). In some instances, the subject has an Expanded Disability Status Scale (EDSS) score of from 0 to 5.5 prior to treatment with the anti-CD20 antibody protein composition described herein.

In some instances, the autoimmune disease is lupus, or Systemic lupus erythematosus (SLE). For example, provided herein is a method of treating an RMS in a subject in need thereof, by administering to the subject an effective amount of an anti-CD20 antibody protein composition of the present disclosure. In some instances, the lupus is systemic lupus erythematosus (SLE). In some instances, the lupus is cutaneous lupus erythematosus or lupus of the skin (e.g., acute cutaneous lupus, chronic cutaneous lupus erythematosus or discoid lupus erythematosus (DLE), and/or subacute cutaneous lupus erythematosus). In some instances, the lupus is drug-induced lupus erythematosus (e.g., lupus induced by the use of one or more of Hydralazine (e.g., used to treat high blood pressure or hypertension), Procainamide (e.g., used to treat irregular heart rhythms), Isoniazid (e.g., used to treat tuberculosis), Minocycline (e.g., used to treat acne), and anti-TNF (e.g., used to treat rheumatoid arthritis, psoriatic arthritis and spondyloarthropathy)). In some instances, the lupus is neonatal lupus.

In some instances, the autoimmune disease is rheumatoid arthritis (RA). For example, provided herein is a method of treating RA in a subject in need thereof, by administering to the subject an effective amount of an anti-CD20 antibody protein composition of the present disclosure. In some instances, the RA is rheumatoid factor positive RA or seropositive RA. In some instances, the RA is rheumatoid factor negative RA or seronegative RA.

In some instances, the autoimmune disease is myasthenia gravis (MG). For example, provided herein is a method of treating myasthenia gravis in a subject in need thereof, by administering to the subject an effective amount of an anti-CD20 antibody protein composition of the present disclosure. Myasthenia gravis (MG) is an autoimmune neuromuscular disorder characterized by weakness and fatigability of skeletal muscles. It is primarily mediated by autoantibodies against acetylcholine receptors (AChR) or muscle-specific kinase (MuSK) at the neuromuscular junction. Thus, a subject treated by the present methods can have positive serologic test for anti-AChR antibody and/or anti-MuSK antibody. In some instances, the MG is oculobulbar MG. In some instances, the MG is bulbar MG. In some instances, the MG is generalized MG.

In some instances, the autoimmune disease is chronic inflammatory demyelinating polyneuropathy (CIDP). For example, provided herein is a method of treating CIDP in a subject in need thereof, by administering to the subject an effective amount of an anti-CD20 antibody protein composition of the present disclosure.

Also provided herein are methods of treating and/or preventing one or more of the following conditions (or diseases) in a subject in need thereof, by administering to the subject an effective amount of an anti-CD20 antibody protein composition of the present disclosure: Chronic inflammatory demyelinating polyneuropathy (CIDP); Myositis; Lupus Nephritis; other forms of MS-PPMS, SPMS; Myasthenia Gravis (MG); Antiphospholipid Syndrome; Thrombotic Thrombocytopenia Purpura (TTP); Ulcerative Colitis; Minimal Change Nephrotic Syndrome (MCNS); Aplastic Anemia; Autoimmune Glomerulopathies; Rheumatoid Arthritis (RA); Interstitial Lung Disease; Myasthenia Gravis (MG); Subepidermal Autoimmune Blistering Disease; Pulmonary Infectious Diseases; Acquired Hemophilia; Refractory Mixed Cryoglobulinemia; Primary Immune Thrombocytopenia; Graft Versus Host Disease (GVHD); Autoimmune Blistering Disease; Anti-Myelin Associated Glycoprotein (MAG) Polyneuropathy; Granulomatosis with Pollyangiitis (GPA); Neuromyelitis Optica; Systemic Lupus Erythematosus; Pemphigus; Post-Transplant Lymphoproliferative Disorders; Autoimmune Hemolytic Anemia; Cerebral Vasculitis; Microscopic Polyangiitis (MPA); and Idiopathic Nephritic Syndrome.

### Cancers

Also provided herein are methods of treating a cancer in a subject in need thereof, by administering to the subject an effective amount of an anti-CD20 antibody protein composition as described herein. In some embodiments, the cancer is a bladder cancer, a breast cancer, a cervical cancer, a colorectal cancer, a gynecologic cancer (i.e., cervical, ovarian, uterine, vaginal, or vulvar cancer), head and neck cancer, kidney cancer, liver cancer, lung cancer, lymphoma, mesothelioma, myeloma, prostate cancer, skin cancer, or thyroid cancer.

Also provided herein are methods of treating a disease or disorder associated with excessive B-cell proliferation in a subject in need thereof, by administering to the subject an effective amount of an anti-CD20 antibody protein composition as described herein. In some embodiments, the disease or disorder associated with excessive B-cell proliferation is a hematological cancer. In some embodiments, the hematological cancer is lymphoma, leukemia, or myeloma. In some embodiments, the hematological cancer is selected from B-cell lymphoma, acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, or primary central nervous system lymphoma (PCNSL). In various aspects of the method, the anti-CD20 antibody protein composition as described herein comprises anti-CD20 antibodies, which are IgG1 antibodies comprising heavy chains each comprising the amino acid sequence of SEQ ID NO: 1, and light chains each comprising the amino acid sequence of SEQ ID NO:2.

In certain embodiments, the hematological cancer is B-cell lymphoma. In some embodiments, the B-cell lymphoma is relapsed or refractory. In certain embodiments, the hematological cancer is non-Hodgkin's lymphoma (NHL). In some embodiments, the NHL is relapsed or refractory. In certain embodiments, the hematological cancer is Waldenstrom's macroglobulinemia (WM). In some embodiments, the WM is relapsed or refractory. In certain embodiments, the hematological cancer is marginal zone lymphoma (MZL). In some embodiments, the MZL is relapsed or refractory. In certain embodiments, the hematological cancer is chronic lymphocytic leukemia (CLL). In some embodiments, the CLL is relapsed or refractory. In certain embodiments, the hematological cancer is small lymphocytic lymphoma (SLL). In some embodiments, the SLL is relapsed or refractory. In certain embodiments, the hematological cancer is primary central nervous system lymphoma (PCNSL). In some embodiments, the PCNSL is relapsed or refractory.

In certain embodiments, the method of treating a hematological cancer comprises administering to the subject the anti-CD20 antibody protein composition as described herein by intravenous infusion. In some embodiments, the intravenous infusion comprises: a) an infusion comprising about 900 mg of the anti-CD20 antibody protein composition as described herein on days 1 and 2 (split into a 150 mg dose on day 1 and a 750 mg dose on day 2), day 8, and day 15 of Cycle 1 (each Cycle being 28 days); day 1 of Cycles 2-6; and day 1 of every 3 cycles after Cycle 6. In some embodiments, the hematological cancer is CLL.

### Subject

A subject who is treated by the present methods can be a human. The human subject can be an adult.

In some instances, the subject is diagnosed with one or more autoimmune diseases (e.g., one or more of MS, lupus, RA, myasthenia gravis, and CIDP) prior to treatment with the present methods. In some embodiments, the subject shows one or more symptoms of an autoimmune diseases (e.g., one or more of MS, lupus, RA myasthenia gravis, and CIDP) prior to treatment with the present methods. In some embodiments, the subject has experienced at least one relapse in the previous year prior to treatment or two relapses in the previous two years prior to treatment. In some embodiments, the subject has or had the presence of a T1 gadolinium (Gd)-enhancing lesion in the previous year prior to treatment with the anti-CD20 antibody protein composition as described herein. In some embodiments, the subject has an Expanded Disability Status Scale (EDSS) score of from 0 to 5.5 prior to treatment with the anti-CD20 antibody protein composition as described herein. In some embodiments, the subject is neurologically stable for >30 days prior to Day 1 (i.e., prior to start of the treatment). In some embodiments, the subject has IgG>4.0 g/L at screening (e.g., prior to start of treatment).

In certain embodiments of the method, the subject has not been treated with a non-steroid therapy for MS in the previous five years prior to treatment with the anti-CD20 antibody protein composition as described herein. In some embodiments, the subject is naïve to treatment for MS. In certain embodiments, the subject is negative for hepatitis B virus (HBV). In some embodiments, the subject is negative for hepatitis B virus surface antigen (HBsAg). In some embodiments, the subject is negative for anti-hepatitis B virus core antibodies. In certain embodiments, the subject has not been immunized with a vaccine for at least 2 weeks or at least 4 weeks prior to treatment with the anti-CD20 antibody protein composition as described herein.

In certain embodiments of the method, the subject is pre-medicated with an amount of a corticosteroid about 30 to about 60 minutes prior to administration of the anti-CD20 antibody protein composition as described herein. In some embodiments, the corticosteroid is methylprednisone or a corticosteroid bioequivalent thereto. In some embodiments, the amount of a corticosteroid is 100 mg methylprednisone. In some embodiments, the corticosteroid is dexamethasone or a corticosteroid bioequivalent thereto. In some embodiments, the amount of a corticosteroid is from 10 to 20 mg dexamethasone. In some embodiments, the corticosteroid is administered intravenously. In some embodiments, the corticosteroid is administered orally. In some embodiments, the subject is pre-medicated with a corticosteroid prior to the first administration only and not prior to subsequent administrations. In certain embodiments, the subject is pre-medicated with an amount of an antipyretic about 30 to about 60 minutes prior to administration of the anti-CD20 antibody protein composition as described herein. In some embodiments, the antipyretic is acetaminophen or an antipyretic bioequivalent thereto. In some embodiments, the antipyretic is administered intravenously. In some embodiments, the antipyretic is administered orally. In some embodiments, the subject is pre-medicated with an anti-pyretic prior to the first administration only and not prior to subsequent administrations. In some embodiments, the subject is pre-medicated with an amount of an antihistamine about 30 to about 60 minutes prior to administration of the anti-CD20 antibody protein composition as described herein. In some embodiments, the antihistamine is diphenhydramine HCl or an antihistamine bioequivalent thereof. In some embodiments, the amount of an antihistamine is from about 25 to 50 mg diphenhydramine HCl. In some embodiments, the antihistamine is a non-drowsy antihistamine. In some embodiments, the non-drowsy antihistamine is cetirizine. In some embodiments, the amount of non-drowsy antihistamine is about 10 mg cetirizine. In some embodiments, the non-drowsy antihistamine is loratadine. In some embodiments, the amount of non-drowsy antihistamine is about 10 mg loratadine. In some embodiments, the antihistamine is administered intravenously. In some embodiments, the antihistamine is administered orally. In some embodiments, the subject is pre-medicated with an antihistamine prior to the first administration only and not prior to subsequent administrations. In some embodiments, the subject is pre-medicated with an amount of a corticosteroid (as described above) and an amount of an antihistamine (as described above) about 30 to about 60 minutes prior to administration of the anti-CD20 antibody protein composition as described herein. In some embodiments, the subject is pre-medicated with an amount of a corticosteroid (as described above) and an amount of an antihistamine (as described above) prior to the first administration only and not prior to subsequent administrations.

In some embodiments, the method alleviates or delays progression of one or more symptoms of an autoimmune disease in the subject. For example, the method can alleviate or delay progression of one or more symptoms of MS, lupus, RA, myasthenia gravis, and/or CIDP in the subject. In certain embodiments, the method reduces the annualized relapse rate (ARR) in the subject following administration of the anti-CD20 antibody protein composition as described herein. In some embodiments, ARR is the total number of relapse/s for a subject divided by the sum of treatment duration. In some embodiments, ARR is number of Independent Relapse Adjudication Panel (IRAP)-confirmed relapses for a subject per year. In certain embodiments, the method reduces the total number of T1 gadolinium (Gd)-enhancing lesions in the subject following administration of the anti-CD20 antibody protein composition as described herein. In some embodiments, reduction of Gd-enhancing T1 lesions is assessed by MRI scan. In certain embodiments, the method reduces the number of new or enlarging T2 hyperintense lesions in the subject following administration of the anti-CD20 antibody protein composition as described herein. In some embodiments, reduction of the number of new or enlarging T2 hyperintense lesions is assessed by MRI scan. In certain embodiments, the method achieves confirmed disability progression in the subject following administration of the anti-CD20 antibody protein composition as described herein. In some embodiments, confirmation of disability progression comprises an increase of greater than or equal to 1.0 point from a baseline EDSS score of the subject attributable to MS, wherein said baseline EDSS score is 5.5 or less. In some embodiments, confirmation of disability progression comprises an increase of greater than or equal to 0.5 point from a baseline EDSS score of the subject attributable to MS, wherein said baseline EDSS score is greater than 5.5.

In some instances, the subject is diagnosed with one or more cancers prior to treatment with the present methods. In some instances, the subject shows one or more symptoms of a cancer prior to treatment with the present methods. In some instances, the subject has experienced at least one relapse in the previous year prior to treatment or two relapses in the previous two years prior to treatment. In some instances, the methods described herein alleviate or delay progression of one or more symptoms of a cancer in the subject.

SEQUENCE TABLE

| SEQ | | |
|---|---|---|
| ID | | |
| NO: | Description | Sequence |
| 1 | Heavy chain | QAYLQQSGAE LVRPGASVKM SCKASGYTFT SYNMHWVKQT PRQGLEWIGG |
| | | IYPGNGDTSY NQKFKGKATL TVGKSSSTAY MQLSSLTSED SAVYFCARYD |
| | | YNYAMDYWGQ GTSVTVSSAS TKGPSVFPLA PSSKSTSGGT AALGCLVKDY |
| | | FPEPVTVSWN SGALTSGVHT FPAVLQSSGL YSLSSVVTVP SSSLGTQTYI |
| | | CNVNHKPSNT KVDKKVEPKS CDKTHTCPPC PAPELLGGPS VFLFPPKPKD |
| | | TLMISRTPEV TCVVVDVSHE DPEVKFNWYV DGVEVHNAKT KPREEQYNST |
| | | YRVVSVLTVL HQDWLNGKEY KCKVSNKALP APIEKTISKA KGQPREPQVY |
| | | TLPPSRDELT KNQVSLTCLV KGFYPSDIAV EWESNGQPEN NYKTTPPVLD |
| | | SDGSFFLYSK LTVDKSRWQQ GNVFSCSVMH EALHNHYTQK SLSLSPGK |
| 2 | Light chain | QIVLSQSPAI LSASPGEKVT MTCRASSSVS YMHWYQQKPG SSPKPWIYAT |
| | | SNLASGVPAR FSGSGSGTSY SFTISRVEAE DAATYYCQQW TFNPPTFGGG |
| | | TRLEIKRTVA APSVFIFPPS DEQLKSGTAS VVCLLNNFYP REAKVQWKVD |
| | | NALQSGNSQE SVTEQDSKDS TYSLSSTLTL SKADYEKHKV YACEVTHQGL |
| | | SSPVTKSFNR GEC |
| 3 | Variable | GYTFTSY |
| | heavy chain | |
| | (VH) CDR1 | |
| 4 | Variable | YPGNGD |
| | heavy chain | |
| | (VH) CDR2 | |
| 5 | Variable | YDYNYAMDY |
| | heavy chain | |
| | (VH) CDR3 | |
| 6 | Variable | QAYLQQSGAE LVRPGASVKM SCKASGYTFT SYNMHWVKQT PRQGLEWIGG |
| | heavy chain | IYPGNGDTSY NQKFKGKATL TVGKSSSTAY MQLSSLTSED SAVYFCARYD |
| | (VH) | YNYAMDYWGQ GTSVTVSS |
| 7 | Constant | ASTKGPSVFP LAPSSKSTSG GTAALGCLVK DYFPEPVTVS WNSGALTSGV |
| | heavy chain | HTFPAVLQSS GLYSLSSVVT VPSSSLGTQT YICNVNHKPS NTKVDKKVEP |
| | | KSCDKTHTCP PCPAPELLGG PSVFLFPPKP KDTLMISRTP EVTCVVVDVS |
| | | HEDPEVKFNW YVDGVEVHNA KTKPREEQYN STYRVVSVLT VLHQDWLNGK |
| | | EYKCKVSNKA LPAPIEKTIS KAKGQPREPQ VYTLPPSRDE LTKNQVSLTC |
| | | LVKGFYPSDI AVEWESNGQP ENNYKTTPPV LDSDGSFFLY SKLTVDKSRW |
| | | QQGNVFSCSV MHEALHNHYT QKSLSLSPGK |
| 8 | Variable | RASSSVSYMH |
| | light chain | |
| | (VL) CDR1 | |
| 9 | Variable | ATSNLAS |
| | light chain | |
| | (VL) CDR2 | |
| 10 | Variable | QQWTFNPPT |
| | light chain | |
| | (VL) CDR3 | |
| 11 | Variable | QIVLSQSPAI LSASPGEKVT MTCRASSSVS YMHWYQQKPG SSPKPWIYAT |
| | light chain | SNLASGVPAR FSGSGSGTSY SFTISRVEAE DAATYYCQQW TFNPPTFGGG |
| | (VL) | TRLEIKR |
| 12 | Constant | TVAAPSVFIF PPSDEQLKSG TASVVCLLNN FYPREAKVQW KVDNALQSGN |
| | light chain | SQESVTEQDS KDSTYSLSST LTLSKADYEK HKVYACEVTH QGLSSPVTKS |
| | | FNRGEC |
| 13 | Modified | pGlu Ala Tyr Leu Gln Gln Ser Gly Ala Glu Leu Val Arg Pro |
| | heavy chain | Gly Ala Ser Val Lys Met Ser Cys Lys Ala Ser Gly Tyr Thr |
| | (substituti | Phe Thr Ser Tyr Asn Met His Trp Val Lys Gln Thr Pro Arg |
| | on of | Gln Gly Leu Glu Trp Ile Gly Gly Ile Tyr Pro Gly Asn Gly |
| | glutamine | Asp Thr Ser Tyr Asn Gln Lys Phe Lys Gly Lys Ala Thr Leu |
| | at position | Thr Val Gly Lys Ser Ser Ser Thr Ala Tyr Met Gln Leu Ser |
| | 1 with | Ser Leu Thr Ser Glu Asp Ser Ala Val Tyr Phe Cys Ala Arg |
| | pyroglutamate) | Tyr Asp Tyr Asn Tyr Ala Met Asp Tyr Trp Gly Gln Gly Thr |
| | | Ser Val Thr Val Ser Ser Ala Ser Thr Lys Gly Pro Ser Val |
| | | Phe Pro Leu Ala Pro Ser Ser Lys Ser Thr Ser Gly Gly Thr |
| | | Ala Ala Leu Gly Cys Leu Val Lys Asp Tyr Phe Pro Glu Pro |
| | | Val Thr Val Ser Trp Asn Ser Gly Ala Leu Thr Ser Gly Val |
| | | His Thr Phe Pro Ala Val Leu Gln Ser Ser Gly Leu Tyr Ser |
| | | Leu Ser Ser Val Val Thr Val Pro Ser Ser Ser Leu Gly Thr |
| | | Gln Thr Tyr Ile Cys Asn Val Asn His Lys Pro Ser Asn Thr |
| | | Lys Val Asp Lys Lys Val Glu Pro Lys Ser Cys Asp Lys Thr |
| | | His Thr Cys Pro Pro Cys Pro Ala Pro Glu Leu Leu Gly Gly |
| | | Pro Ser Val Phe Leu Phe Pro Pro Lys Pro Lys Asp Thr Leu |
| | | Met Ile Ser Arg Thr Pro Glu Val Thr Cys Val Val Val Asp |
| | | Val Ser His Glu Asp Pro Glu Val Lys Phe Asn Trp Tyr Val |
| | | Asp Gly Val Glu Val His Asn Ala Lys Thr Lys Pro Arg Glu |
| | | Glu Gln Tyr Asn Ser Thr Tyr Arg Val Val Ser Val Leu Thr |
| | | Val Leu His Gln Asp Trp Leu Asn Gly Lys Glu Tyr Lys Cys |
| | | Lys Val Ser Asn Lys Ala Leu Pro Ala Pro Ile Glu Lys Thr |
| | | Ile Ser Lys Ala Lys Gly Gln Pro Arg Glu Pro Gln Val Tyr |
| | | Thr Leu Pro Pro Ser Arg Asp Glu Leu Thr Lys Asn Gln Val |
| | | Ser Leu Thr Cys Leu Val Lys Gly Phe Tyr Pro Ser Asp Ile |
| | | Ala Val Glu Trp Glu Ser Asn Gly Gln Pro Glu Asn Asn Tyr |
| | | Lys Thr Thr Pro Pro Val Leu Asp Ser Asp Gly Ser Phe Phe |
| | | Leu Tyr Ser Lys Leu Thr Val Asp Lys Ser Arg Trp Gln Gln |
| | | Gly Asn Val Phe Ser Cys Ser Val Met His Glu Ala Leu His |
| | | Asn His Tyr Thr Gln Lys Ser Leu Ser Leu Ser Pro Gly Lys |
| 14 | Modified | pGlu Ile Val Leu Ser Gln Ser Pro Ala Ile Leu Ser Ala Ser |
| | light chain | Pro Gly Glu Lys Val Thr Met Thr Cys Arg Ala Ser Ser Ser |
| | (substituti | Val Ser Tyr Met His Trp Tyr Gln Gln Lys Pro Gly Ser Ser |
| | on of | Pro Lys Pro Trp Ile Tyr Ala Thr Ser Asn Leu Ala Ser Gly |
| | glutamine | Val Pro Ala Arg Phe Ser Gly Ser Gly Ser Gly Thr Ser Tyr |
| | at position | Ser Phe Thr Ile Ser Arg Val Glu Ala Glu Asp Ala Ala Thr |
| | 1 with | Tyr Tyr Cys Gln Gln Trp Thr Phe Asn Pro Pro Thr Phe Gly |
| | pyroglutamate) | Gly Gly Thr Arg Leu Glu Ile Lys Arg Thr Val Ala Ala Pro |
| | | Ser Val Phe Ile Phe Pro Pro Ser Asp Glu Gln Leu Lys Ser |
| | | Gly Thr Ala Ser Val Val Cys Leu Leu Asn Asn Phe Tyr Pro |
| | | Arg Glu Ala Lys Val Gln Trp Lys Val Asp Asn Ala Leu Gln |
| | | Ser Gly Asn Ser Gln Glu Ser Val Thr Glu Gln Asp Ser Lys |
| | | Asp Ser Thr Tyr Ser Leu Ser Ser Thr Leu Thr Leu Ser Lys |
| | | Ala Asp Tyr Glu Lys His Lys Val Tyr Ala Cys Glu Val Thr |
| | | His Gln Gly Leu Ser Ser Pro Val Thr Lys Ser Phe Asn Arg |
| | | Gly Glu Cys |
| 15 | Modified | QAYLQQSGAE LVRPGASVKM SCKASGYTFT SYNMHWVKQT PRQGLEWIGG |
| | heavy chain | IYPGNGDTSY NQKFKGKATL TVGKSSSTAY MQLSSLTSED SAVYFCARYD |
| | (deletion | YNYAMDYWGQ GTSVTVSSAS TKGPSVFPLA PSSKSTSGGT AALGCLVKDY |
| | of the C- | FPEPVTVSWN SGALTSGVHT FPAVLQSSGL YSLSSVVTVP SSSLGTQTYI |
| | terminal | CNVNHKPSNT KVDKKVEPKS CDKTHTCPPC PAPELLGGPS VFLFPPKPKD |
| | lysine) | TLMISRTPEV TCVVVDVSHE DPEVKFNWYV DGVEVHNAKT KPREEQYNST |
| | | YRVVSVLTVL HQDWLNGKEY KCKVSNKALP APIEKTISKA KGQPREPQVY |
| | | TLPPSRDELT KNQVSLTCLV KGFYPSDIAV EWESNGQPEN NYKTTPPVLD |
| | | SDGSFFLYSK LTVDKSRWQQ GNVFSCSVMH EALHNHYTQK SLSLSPG |
| 16 | Light chain | QIVLSQSPAI LSASPGEKVT MTCRASSSVS YMHWYQQKPG SSPKPWIYAT |
| | with one | SNLASGVPAR FSGSGSGTSY SFTISRVEAE DAATYYCQQW TFNPPTFGGG |
| | amino acid | TRLEINRTVA APSVFIFPPS DEQLKSGTAS VVCLLNNFYP REAKVQWKVD |
| | mismatch at | NALQSGNSQE SVTEQDSKDS TYSLSSTLTL SKADYEKHKV YACEVTHQGL |
| | position | SSPVTKSFNR GEC |
| | 106 | |
| 17 | Post- | MSCK |
| | translational | |
| | modification | |
| | site HC | |
| | (20-23) | |
| 18 | Post- | ASGYTFTSYNMHWVK |
| | translational | |
| | modification | |
| | site HC | |
| | (24-38) | |
| 19 | Post- | QTPRQGLEWIGGIYPGNGDTSYNQK |
| | translational | |
| | modification | |
| | site HC | |
| | (39-63) | |
| 20 | Post- | GKATLTVGK |
| | translational | |
| | modification | |
| | site HC | |
| | (66-74) | |
| 21 | Post- | SSSTAYMQLSSLTSEDSAVYFCARYDYNYAMDYWGQGTSVTVSSASTK |
| | translational | |
| | modification | |
| | site HC | |
| | (75-122) | |
| 22 | Post- | DYFPEPVTVSWNSGALTSGVHTFPAVLQSSGLYSLSSVVTVPSSSLGTQTYICNVNH |
| | translational | K |
| | modification | |
| | site HC | |
| | (149-206) | |
| 23 | Post- | PKDTLMISRTPEVTCVVVDVSHEDPEVK |
| | translational | |
| | modification | |
| | site HC | |
| | (248-275) | |
| 24 | Post- | DTLMISRTPEVTCVVVDVSHEDPEVK |
| | translational | |
| | modification | |
| | site HC | |
| | (250-275) | |
| 25 | Post- | FNWYVDGVEVHNAK |
| | translational | |
| | modification | |
| | site HC | |
| | (276-289) | |
| 26 | Post- | VSNKALPAPIEK |
| | translational | |
| | modification | |
| | site HC | |
| | (324-335) | |
| 27 | Post- | NQVSLTCLVK |
| | translational | |
| | modification | |
| | site HC | |
| | (362-371) | |
| 28 | Post- | GFYPSDIAVEWESNGQPENNYK |
| | translational | |
| | modification | |
| | site HC | |
| | (372-393) | |
| 29 | Post- | SRWQQGNVFSCSVMHEALHNHYTQK |
| | translational | |
| | modification | |
| | site HC | |
| | (416-440) | |
| 30 | Post- | VTMTCRASSSVSYMHWYQQK |
| | translational | |
| | modification | |
| | site LC | |
| | (19-38) | |
| 31 | Post- | SGTASVVCLLNNFYPREAK |
| | translational | |
| | modification | |
| | site LC | |
| | (126-144) | |
| 32 | Post- | VDNALQSGNSQESVTEQDSK |
| | translational | |
| | modification | |
| | site LC | |
| | (149-168) | |
| 33 | Peptide for | TKPREEQYNSTYRVVSVLTVLHQDWLNGK |
| | determining | |
| | glycosylation | |
| | following | |
| | Lys-C | |
| | digestion | |
| | HC (290-318) | |
| 34 | Peptide for | DIAVEWESNGQPENNYKTTPPVL |
| | determining | |
| | deamidation | |
| | following | |
| | Asn-N | |
| | digestion | |
| | HC (377-399) | |
| 35 | Nucleic | caggcttatctacagcagtctggggctgagctggtgaggcctggggcctcagtgaag |
| | acid | atgtcctgcaaggcttctggctacacatttaccagttacaatatgcactgggtaaag |
| | sequence of | cagacacctagacagggcctggaatggattggaggtatttatccaggaaatggtgat |
| | the heavy | acttcctacaatcagaagttcaagggcaaggccacactgactgtaggcaaatcctcc |
| | chain | agcacagcctacatgcagctcagcagcctgacatctgaagactctgcggtctatttc |
| | | tgtgcaagatatgactacaactatgctatggactactggggtcaaggaacctcagtc |
| | | accgtctcctcagcctccaccaagggcccatcggtcttccccctggcaccctcctcc |
| | | aagagcacctctgggggcacagcggccctgggctgcctggtcaaggactacttcccc |
| | | gaaccggtgacggtgtcgtggaactcaggcgccctgaccagcggcgtgcacaccttc |
| | | ccggctgtcctacagtcctcaggactctactccctcagcagcgtggtgaccgtgccc |
| | | tccagcagcttgggcacccagacctacatctgcaacgtgaatcacaagcccagcaac |
| | | accaaggtggacaagaaagttgagcccaaatcttgtgacaaaactcacacatgccca |
| | | ccgtgcccagcacctgaactcctggggggaccgtcagtcttcctcttccccccaaaa |
| | | cccaaggacaccctcatgatctcccggacccctgaggtcacatgcgtggtggtggac |
| | | gtgagccacgaagaccctgaggtcaagttcaactggtacgtggacggcgtggaggtg |
| | | cataatgccaagacaaagccgcgggaggagcagtacaacagcacgtaccgtgtggtc |
| | | agcgtcctcaccgtcctgcaccaggactggctgaatggcaaggagtacaagtgcaag |
| | | gtctccaacaaagccctcccagcccccatcgagaaaaccatctccaaagccaaaggg |
| | | cagccccgagaaccacaggtgtacaccctgcccccatcccgggatgagctgaccaag |
| | | aaccaggtcagcctgacctgcctggtcaaaggcttctatcccagcgacatcgccgtg |
| | | gagtgggagagcaatgggcagccggagaacaactacaagaccacgcctcccgtgctg |
| | | gactccgacggctccttcttcctctacagcaagctcaccgtggacaagagcaggtgg |
| | | cagcaggggaacgtcttctcatgctccgtgatgcatgaggctctgcacaaccactac |
| | | acgcagaagagcctctccctgtctccgggtaaa |
| 36 | Nucleic | caaattgttctctcccagtctccagcaatcctgtctgcatctccaggggagaaggtc |
| | acid | acaatgacttgcagggccagctcaagtgtaagttacatgcactggtaccagcagaag |
| | sequence of | ccaggatcctcccccaaaccctggatttatgccacatccaacctggcttctggagtc |
| | the light | cctgctcgcttcagtggcagtgggtctgggacctcttattctttcacaatcagcaga |
| | chain | gtggaggctgaagatgctgccacttattactgccagcagtggacttttaacccaccc |
| | | acgttcggaggggggaccaggctggaaataaaacggactgtggctgcaccaagtgtc |
| | | ttcatcttcccgccatctgatgagcagttgaaatctggaactgcctctgttgtgtgc |
| | | ctgctgaataacttctatcccagagaggccaaagtacagtggaaggtggataacgcc |
| | | ctccaatcgggtaactcccaggagagtgtcacagagcaggacagcaaggacagcacc |
| | | tacagcctcagcagcaccctgacgctgagcaaagcagactacgagaaacacaaagtc |
| | | tacgcctgcgaagtcacccatcagggcctgagctcgcccgtcacaaagagcttcaac |
| | | aggggagagtgt |
| 37 | Epitope on | IRAHT |
| | CD20 | |
| 38 | Epitope on | EPAN |
| | CD20 | |
| 39 | HC1-19 | QAYLQQSGAELVRPGASVK |
| 40 | HC 43-63 | QGLEWIGGIYPGNGDTSYNQK |
| 41 | HC 99-122 | YDYNYAMDYWGQGTSVTVSSASTK |
| 42 | HC 250-256 | DTLMISR |
| 43 | HC 290-302 | TKPREEQYNSTYR |
| 44 | HC 303-318 | VVSVLTVLHQDWLNGK |
| 45 | HC 324-327 | VSNK |
| 46 | HC 418-440 | WQQGNVFSCSVMHEALHNHYTQK |
| 47 | HC 441-447 | SLSLSPGK |
| 48 | LC 1-18 | QIVLSQSPAILSASPGEK |
| 49 | LC 126-141 | SGTASVVCLLNNFYPR |
| 50 | LC 77-102 | VEAEDAATYYCQQWTFNPPTFGGGTR |

The following examples are offered by way of illustration, and not by way of limitation.

### EXAMPLES

The invention is further described in the following examples, which do not limit the scope of the invention described in the claims.

### Example 1. Manufacturing Process

Manufacturing of ublituximab high concentration (200 mg/mL) begins with the drug substance intermediate (diluted UFDF Pool) from ublituximab manufacturing as the starting material. Steps to manufacture the starting material is described in detail in WO 2023/235762, the entire contents of which are incorporated herein by reference.

The X and Y manufacturing processes that were utilized for manufacturing ublituximab high concentration (200 mg/mL) are illustrated in FIG. 1. The buffers, operational parameters and performance parameters that were utilized for both processes are detailed in Tables 1-6. The differences in the processes were supported by Process Y development. Differences implemented in the GMP manufacturing of Process Y supported by this process development work included increasing the upper limit for TFF membrane loading factor from 300 to 1000 g/m² and modifying the formulation strategy to account for differences in stabilization formulation buffer composition (the drug substance formulation has not been altered). Additionally, the Acceptable Range for final protein concentration after recovery from the TFF system was changed to account for the change in stabilization buffer formulation. Differences in final formulation stabilization buffer are described in Table 1 and Table 2.

For both processes, an ultrafiltration step concentrated the product, followed by a diafiltration step to exchange the product into the formulation buffer (without polysorbate 80 (PS80)). After diafiltration, the product was concentrated via another ultrafiltration step (UF2) and recovered from the TFF system. After UFDF recovery, formulation with PS80 was completed and the product was diluted to the desired product concentration to obtain ublituximab high concentration (200 mg/mL) formulated bulk drug substance. The drug substance was nominally formulated in 225 mM Arginine, 245 mM Glutamate, 0.05% polysorbate 80, pH 5.5. The excipient concentrations in the drug substance vary from the nominal values due to the Gibbs-Donnan effect (Kannan et al., 2023). After 0.2 μm filtration and filling into Celsius bags (e.g., 6 L Celsius bags), the ublituximab high concentration (200 mg/mL) drug substance was frozen and stored at ≤−60° C.

Ublituximab high concentration (200 mg/mL) manufacturing was performed using approved batch records according to cGMPs. In addition to instructions for performance of each unit operation, the batch records also included operational ranges and controls. In-process monitoring was performed to ensure acceptable performance of the manufacturing process.

The sections below provide a more detailed description of each of the unit operations and provide a summary of operational controls and in-process monitoring that were performed.

### Buffer Preparation

Buffers were prepared with water for injection (WFI) and filtered with a 0.2 μm filter. All buffers were prepared and stored at room temperature. Buffer compositions and performance parameters for Process X and Process Y are provided in Table 1 and Table 2, respectively.

The Process Y TFF system was equilibrated with the diafiltration buffer (225 mM arginine, 245 mM glutamate, pH 5.5) instead of the equilibration buffer used in Process X (25 mM sodium citrate, 154 mM NaCl, pH 6.5). The Process Y used a stabilization formulation buffer with 10% v/v PS80 versus the 1% v/v PS80 buffer used in Process X. The target concentration of 0.05% PS80 in the final drug substance was unchanged for Process Y.

TABLE 1
Process X Buffer Performance Parameters

| | | Target (Acceptable Range) | | | | Action Limit | |
|---|---|---|---|---|---|---|---|
| | | | Cond, | Osmo., | Hold | Endotoxin, | Bioburden, |
| Purpose | Composition | pH | mS/cm | mOsm/kg | Time | EU/mL | CFU/10 mL |
| UFDF (TFF | 25 mM Sodium | 6.50 | Report | 340-380 | ≤28 d | Report | Report |
| system) | Citrate, 154 mM | (6.40-6.60) | Results | | | Results | Results |
| Equilibration | NaCl, pH 6.5 | | | | | | |
| Diafiltration | 225 mM | 5.50 | Report | Report | ≤28 d | Report | Report |
| | Arginine, 245 | (5.30-5.70) | Results | Results | | Results | Results |
| | mM Glutamate, | | | | | | |
| | pH 5.5 | | | | | | |
| Final | 225 mM | 5.50 | Report | Report | ≤28 d | Report | Report |
| Formulation | Arginine, 245 | (5.30-5.70) | Results | Results | | Results | Results |
| Stabilization | mM Glutamate, | | | | | | |
| | 1% Polysorbate | | | | | | |
| | 80, pH 5.5 | | | | | | |
| Final | 225 mM | 5.50 | Report | Report | ≤28 d | Report | Report |
| Formulation | Arginine, 245 | (5.30-5.70) | Results | Results | | Results | Results |
| Dilution | mM Glutamate, | | | | | | |
| | 0.05% | | | | | | |
| | Polysorbate 80, | | | | | | |
| | pH 5.5 | | | | | | |

Cond = conductivity;
osmo = osmolality;
d = days

TABLE 2
Process Y Buffer Performance Parameters

| | | | | | | Action Limit | |
|---|---|---|---|---|---|---|---|
| | | Target (Acceptable Range) | | | | | Bioburden, |
| | | | Cond, | Osmo., | Hold | Endotoxin, | CFU/10 |
| Purpose | Composition | pH | mS/cm | mOsm/kg | Time | EU/mL | mL |
| UFDF (TFF | 225 mM arginine, | 5.5 | Report | Report | ≤34 d^{a} | Report | Report |
| System) | 245 mM glutamate, | (5.4- | Results | Results | | Results | Results |
| Equilibration & | pH 5.5 | 5.6) | | | | | |
| Diafiltration | | | | | | | |
| Final | 225 mM arginine, | 5.5 | Report | Report | ≤14 d^{a} | Report | Report |
| Formulation | 245 mM glutamate, | (5.4- | Results | Results | | Results | Results |
| Stabilization | 10% polysorbate 80, | 5.6) | | | | | |
| | pH 5.5 | | | | | | |

^{a}Hold time is facility specific
Cond = conductivity;
osmo = osmolality;
d = days

### Thawing

The ublituximab drug substance intermediate (diluted UFDF Pool; high concentration starting material) was stored frozen at ≤−35° C. for up to 6 months. This included shipment on dry ice between manufacturers. For forward processing, the diluted UFDF Pool was thawed at room temperature and 0.2 μm-filtered. This intermediate was either used immediately for manufacturing or stored at 2-8° C. for up to an additional 6 months.

### Ultrafiltration (UF) and Diafiltration (DF)

The UFDF process used tangential flow filtration (TFF) with 30 kDa nominal molecular weight cut-off (NMWCO) membranes. Prior to product introduction, the TFF system and membranes were sanitized with 0.5M NaOH, flushed with WFI, and flushed/equilibrated with equilibration buffer. Then ublituximab drug substance intermediate (diluted UFDF Pool; high concentration starting material) was introduced into the TFF system as the UFDF Load. The product is referred to as the retentate during operations. An initial ultrafiltration step (UF1) was used to concentrate the product. After UF1 was complete, a diafiltration (DF) step was used to buffer-exchange the concentrated product into the appropriate formulation buffer for ublituximab high concentration (200 mg/mL). After DF was complete, the product was concentrated via another ultrafiltration step (UF2) and recovered from the TFF system. This allowed for formulation while maintaining the target protein concentration for drug substance.

Operational and performance parameters for this step are provided in Table 3 and Table 4, respectively.

TABLE 3
Ultrafiltration and Diafiltration (UFDF) Operational Parameters

| | Acceptable Range (AR) | |
|---|---|---|
| Process Parameter | Process X | Process Y |
| TFF Membrane Loading factor, | ≤300 | ≤1000 |
| g protein /m² membrane | | |
| Final protein concentration after | ≥210 | ≥205 |
| recovery from the TFF system, mg/mL | | |

TABLE 4
Ultrafiltration and Diafiltration (UFDF) Performance Parameters

| | Action Limit | |
|---|---|---|
| Process Parameter | Process X | Process Y |
| Membrane equilibration bioburden, | Report Results | TAMC: ≤5 |
| CFU/10 mL | | TYMC: ≤5 |
| Membrane equilibration endotoxin, | Report Results | ≤2.0 |
| EU/mL | | |
| UFDF load, bioburden, CFU/10 mL | Report Results | TAMC: ≤1 |
| | | TYMC: ≤1 |
| UFDF load, endotoxin, EU/mL | Report Results | ≤2.0 |

### Formulation, Filtration, Filling and Storage

The formulation step included the addition of concentrated polysorbate 80 (PS80) in formulation buffer to achieve the final drug substance formulation. The stabilization buffer's composition differed between Process X (1% v/v PS80 concentration) and Process Y (10% v/v PS80 concentration), but the additional volume was adjusted to ensure that resulting PS80 concentration in the DS formulation remains the same between the two processes. After the addition of the stabilization buffer, the formulated product pool was diluted to a target of 200 mg/mL with 225 mM arginine, 245 mM glutamate, 0.05% polysorbate 80, pH 5.5.

The formulated bulk drug substance was 0.2 μm-filtered into CELSIUS® FFT bags in a closed, single use system. After filling, the ublituximab high concentration (200 mg/mL) drug substance was frozen and stored at ≤−60° C.

Operational and performance parameters for this step at each site (Site X and Site Y) are provided in Table 5 and Table 6, respectively.

TABLE 5
Formulation, Filtration and Filling Operational Parameters

| | Acceptable Range (AR) | |
|---|---|---|
| Process Parameter | Site X | Site Y |
| Formulation stabilization buffer addition, | 0.05 | 0.005 |
| L buffer/L product pool | | |
| Formulated bulk (pre-filtration) drug | 180-220 | 180-220 |
| substance concentration, mg/mL | | |

TABLE 6
Formulation, Filtration and Filling Performance Parameters

| | Acceptable Range (AR) | |
|---|---|---|
| Process Parameter | Site X | Site Y |
| Filter integrity testing, post-use | Pass | Pass |
| Formulated bulk drug substance (BDS) pool | Report | TAMC: ≤1 |
| (end of hold, pre-filtration) bioburden, | Results | TYMC: ≤1 |
| CFU/10 mL | | |
| Formulated bulk (BDS) pool (end of hold, | Report | ≤2.0 |
| pre-filtration) endotoxin, EU/mL | Results | |

### Materials Used in the Purification Process

The starting material for the manufacture of ublituximab high concentration (200 mg/mL) is the ublituximab diluted UFDF pool. The specifications and results are provided in Table 7. All acceptance criteria were met.

TABLE 7
Ublituximab Diluted UFDF Pool Starting Material Specifications and Results

| | | Batch Results | | |
|---|---|---|---|---|
| | Acceptance | 3174394^{a} used in | 3174389^{a} used in | 3174391^{a} used in |
| Test | Criteria | 2VAC2401CA | 2VAC2402CA | 2VAC2403CA |
| Protein Conc, mg/mL | 27.0 to 41.0 | 31.7 | 31.0 | 32.2 |
| Size Exclusion | ≥97.6 | 98.2 | 98.1 | 98.4 |
| Chromatography (SEC) | | | | |
| Monomer, % | | | | |
| Size Exclusion | ≤2.2 | 1.7 | 1.9 | 1.6 |
| Chromatography (SEC) Total | | | | |
| Aggregates | | | | |
| (Dimer + Aggregates) | | | | |
| Size Exclusion | ≤1.6 | 0.1 | 0.1 | 0.1 |
| Chromatography (SEC) | | | | |
| Fragments, % | | | | |
| Capillary Gel Electrophoresis | ≥91.0 | 93.0 | 93.0 | 93.5 |
| (Non-Reduced) IgG, % | | | | |
| Capillary Gel Electrophoresis | ≤0.7 | 0.1 | 0.2 | 0.1 |
| (Non-Reduced) HMWS, % | | | | |
| Capillary Gel Electrophoresis | ≤2.0 | 1.4 | 1.3 | 1.3 |
| (Non-Reduced) Light Chain, % | | | | |
| Capillary Gel Electrophoresis | ≥97.1 | 97.9 | 98.0 | 98.0 |
| (Reduced) HC + LC, % | | | | |
| Imaged Capillary Isoelectric | 9.3 to 9.5 | 9.3 | 9.3 | 9.3 |
| Focusing (iCIEF) pI | | | | |
| Imaged Capillary Isoelectric | ≤26.0 | 25.4 | 24.5 | 24.4 |
| Focusing (iCIEF) Acidic Peaks, % | | | | |
| Imaged Capillary Isoelectric | ≥49.0 | 50.4 | 51.1 | 53.6 |
| Focusing (iCIEF) Main Peak, % | | | | |
| Imaged Capillary Isoelectric | ≤34.0 | 24.2 | 24.4 | 22.1 |
| Focusing (iCIEF) Basic Peaks, % | | | | |
| Host Cell Protein (HCP) by | ≤20 ng/mg | 5 | 6 | 5 |
| ELISA | protein | | | |
| Residual Protein A | ≤5 ng/mg | <0.2 | <0.2 | <0.2 |
| | protein | | | |
| Residual DNA | ≤5 pg/mg | <0.2 | <0.2 | <0.2 |
| | protein | | | |
| Bioburden, CFU/10 mL^{b} | ≤1 | 0 / 0 | 0 / 0 | 0 / 0 |
| Endotoxin, EU/mL^{b} | ≤2.0 | 0.94 / 0.71 | 0.82 / 0.79 | 0.83 / 0.82 |
| Identity by peptide mapping | Complies with | Complies with | Complies with | Complies with |
| | reference | Reference | Reference | Reference |
| | standard^{c} | | | |

^{a}Batch of Diluted UFDF Pool used to manufacture the named drug substance batch.
^{b}Bioburden and endotoxin were tested at point of use from the load of each cycle.
^{c}“Complies with Reference Standard” and “Complies with Reference” are interchangeable.

Raw materials for drug substance manufacturing operations were procured, received, quarantined, tested, and released by the Manufacturer's Quality unit prior to use in manufacturing. Raw materials for use in GMP manufacturing and quality control testing are procured through qualified vendors/suppliers. Table 8 summarizes the raw materials used in ublituximab high concentration (200 mg/mL) for concentration and formulation operations. Manufacturers are specified for non-compendial raw materials where a change in vendor would require an assessment to assure comparable process performance.

TABLE 8
Additional Raw Materials Used for Concentration
and Formulation Operations

| Raw Material | Quality Standard or Supplier |
|---|---|
| 30 kDa Millipore Pellicon 3 Ultracel | MilliporeSigma |
| (UF membrane) or equivalent | |
| L-Arginine | Ph. Eur., USP |
| L-Glutamic Acid | Ph. Eur., USP |
| Polysorbate 80 | Ph. Eur., NF |

### Example 2. Ublituximab High Concentration (200 mg/mL) Manufacturing Process Development

A detailed manufacturing history for ublituximab producing the drug substance intermediate (diluted UFDF Pool), from which ublituximab high concentration (200 mg/mL) is further processed, is provided in WO 2023/235762, the entire contents of which are incorporated herein by reference. The present Example covers process development activities that were done for ublituximab high concentration (200 mg/mL). A UFDF step concentrates and buffer exchanges the ublituximab drug substance intermediate (diluted UFDF pool). This retentate is then formulated with polysorbate 80 and diluted to a 200 mg/mL drug substance formulation, which is filtered and filled into the DS container. The GMP batches are listed in Table 9.

TABLE 9
Manufacturing Process of ublituximab
high concentration (200 mg/mL)

| Site/Bioreactor | | | Number of |
|---|---|---|---|
| Scale | Process Description | Batches | Batches |
| SBL/15000 L (until | Process intermediate | 2VAC2401CA, | 3 |
| diluted UFDF Pool), | further processed at | 2VAC2402CA, | |
| Process X | Process X to create DS | 2VAC2403CA | |
| (UFDF/Form/Fill) | | | |

### Process Development

Process development (PD) was performed by running three scale-down development batches. PD experiments used 0.11 m² Pellicon 3 Ultracel 30 kDa D screen membranes, and one UFDF cycle was completed for each batch. In comparison, GMP processing used 1.14 m² Pellicon 3 Ultracel 30 kDa D screen membranes and two cycles per batch. The membranes are treated as single use. The UFDF Pool intermediates from the two cycles were then pooled and formulated/filled as one drug substance (DS) batch.

Table 10 provides a comparison of the process parameters Acceptable Ranges (AR) for the PD and GMP runs. A comparison of the process evaluation criteria is shown in Table 11. The results confirmed that the process was successfully developed and modeled the GMP process, with similar process performance across the two steps for all batches. An additional batch (PB #1) was produced at the development-scale as representative material for a supportive stability study.

TABLE 10
Process Development and GMP Process Parameter Comparison

| Process Step | Development AR | GMP AR |
|---|---|---|
| TFF Membrane Loading factor, g | 125-300 | ≤300 |
| protein /m² membrane | | |
| Target Retentate concentration | 55-65 | 70 |
| after UF1, mg/mL | | |
| Diafiltration (DF) Volume, | ≥8 | ≥8 |
| diavolumes | | |
| Final protein concentration after | ≥200 | ≥210 |
| recovery from the TFF system, | | |
| mg/mL | | |
| Formulation Stabilization Buffer | 225 mM Arginine, 245 mM | 225 mM Arginine, 245 mM |
| | Glutamate, 1.0% PS80, pH 5.5 | Glutamate, 1.0% PS80, pH 5.5 |
| Formulation stabilization buffer | 0.05 | 0.05 |
| addition, L buffer/ L product pool | | |
| Target formulated bulk drug | 200 | 200 |
| substance concentration, mg/mL | | |

TABLE 11
Process Development and GMP Process Results Comparison

| | | | | | 2VAC2401CA | 2VAC2402CA | 2VAC2403CA |
|---|---|---|---|---|---|---|---|
| | Process | | | | (GMP1 Cycle | (GMP2 Cycle | (GMP3 Cycle |
| | Parameter | PD Run | PD Run | PD Run | 1/GMP1 | 1/GMP2 | 1/GMP3 |
| Step | or Attribute | #1 | #2 | #3 | Cycle 2) | Cycle 2) | Cycle 2) |
| UFDF^{a} | Ublituximab | 3174395 + 3174396 | 3174395 | 3174393 | 3174394 | 3174389 | 3174391 |
| | Intermediate | | | | | | |
| | Batch | | | | | | |
| | Number | | | | | | |
| | TFF | 231 | 129 | 300 | 207/170 | 206/202 | 217/203 |
| | Membrane | | | | | | |
| | Loading | | | | | | |
| | factor, g | | | | | | |
| | protein/m² | | | | | | |
| | membrane | | | | | | |
| | Retentate | 61 | 67 | 67 | 75/72 | 69/72 | 72/71 |
| | concentration | | | | | | |
| | after UF1, | | | | | | |
| | mg/mL | | | | | | |
| | Diavolumes | 8.0 | 8.0 | 8.0 | 8.4/8.3 | 8.5/8.3 | 8.4/8.5 |
| | Final protein | 211 | 202 | 244 | 230/214 | 252/231 | 242/223 |
| | concentration | | | | | | |
| | after recovery | | | | | | |
| | from the TFF | | | | | | |
| | system, | | | | | | |
| | mg/mL | | | | | | |
| Form'n/ | Formulation | 0.05 | 0.05 | 0.05 | 0.05 | 0.05 | 0.05 |
| Fill | buffer | | | | | | |
| | addition, L | | | | | | |
| | buffer/ | | | | | | |
| | L product | | | | | | |
| | pool | | | | | | |
| | Formulated | 181 | 182 | 178 | 203 | 200 | 203 |
| | bulk (pre- | | | | | | |
| | filtration) | | | | | | |
| | drug | | | | | | |
| | substance | | | | | | |
| | concentration, | | | | | | |
| | mg/mL | | | | | | |

^{a}GMP results represent 2 UFDF cycles which were processed separately and pooled together prior to formulation

### Analytical Comparability

The analytical comparability between ublituximab high concentration (200 mg/mL) and ublituximab IV has been assessed through batch release testing, characterization, and stability. The data demonstrate that ublituximab high concentration (200 mg/mL) is comparable to the ublituximab IV drug substance. In cases where there are concentration- and formulation-related differences such as total protein, pH, and osmolality, all results remain within acceptable levels and control strategies are in place to adequately minimize their potential contribution.

### Batch Release Data Comparison

A comparison of release data for ublituximab high concentration (200 mg/mL) GMP DS batches and commercial ublituximab DS batches is summarized in Table 12. The batch release data show that the ublituximab high concentration (200 mg/mL) is comparable to the ublituximab IV.

TABLE 12
DS Batch Release Data for Ublituximab High Concentration (200 mg/mL) and Ublituximab IV

| | | | Ublituximab High | | Uublituximab IV | |
|---|---|---|---|---|---|---|
| | | | Concentration (200 mg/mL) | | (25 mg/mL) | |
| | | | (N = 3) | | (N = 11) | |
| Test Method | Acceptance Criteria | | Average | Range | Average | Range |
| Opalescence | Slightly opalescent to very | | Opalescent | Opalescent | Opalescent | Opalescent |
| | opalescent (≤Reference | | | | | |
| | Suspension IV) | | | | | |
| Color | Slightly yellow to yellow | | Slightly | Slightly | Slightly | Slightly |
| | (≤Y3) | | yellow | yellow | yellow | yellow |
| Total Protein (A280) | 180-220 | mg/mL | 205 | 202-211 | 25.6 | 25.3-25.8 |
| pH | 5.2-5.7 | | 5.4 | 5.4-5.4 | 6.5 | 6.5-6.6 |
| Osmolality | 400-500 | mOsm/kg | 430 | 429-432 | 358 | 254-361 |
| Peptide Mapping | Complies with reference | | Complies | | Complies | |
| | standard | | | | | |
| Size exclusion- | Monomer, ≥95.0% | | 97.7 | 97.1-98.0 | 98.4^{a} | 98.2-98.5 |
| chromatography | Total Aggregates | | 2.3 | 2.0-2.9 | 1.5^{a} | 1.4-1.8 |
| (SEC) | (Dimer + Aggregates) ≤5.0% | | | | | |
| | Fragments, ≤1.6% | | 0.1 | 0.1 | 0.0^{a} | 0.0-0.0 |
| Capillary gel | Purity, ≥85.0% | | 93.0 | 92.8-93.1 | 94.5 | 93.6-95.9 |
| electrophoresis, | LC, ≤4.0% | | 1.3 | 1.2-1.3 | 1.0 | 0.7-1.3 |
| non-reduced (CGE) | HMWS, ≤3.0% | | 0.4 | 0.3-0.4 | 0.2 | 0.1-0.3 |
| Capillary gel | Purity, ≥94.0% | | 97.9 | 97.9-98.0 | 97.7 | 97.4-98.0 |
| electrophoresis, | | | | | | |
| reduced (CGE) | | | | | | |
| Imaged capillary | pI 9.3-9.5 | | 9.3 | 9.3-9.3 | 9.4 | 9.4-9.4 |
| isoelectric focusing | Acidic, ≤32.0% | | 25.6 | 24.5-26.3 | 23 | 20-25 |
| (iCIEF) | Main, ≥40.0% | | 50.6 | 49.2-53.4 | 54 | 50-56 |
| | Basic, ≤40.0% | | 23.8 | 22.1-24.8 | 23 | 20-27 |
| ADCC potency | 63-177% | | 89 | 84-97 | 120 | 90-163 |
| assay | | | | | | |
| CDC activity by | 74-127% | | 81^{b} | 81^{b} | 93 | 78-116 |
| cell-based bioassay | | | | | | |
| Residual Protein A ^{c} | ≤5 ng/mg protein | | <0.2 | <0.2-<0.2 | <1 | <1-<1 |
| Host cell protein by | ≤20 ng/mg protein | | 5 | 5-6 | 5 | 3-8 |
| ELISA ^{c} | | | | | | |
| Residual DNA ^{c} | ≤5 pg/mg protein | | <0.2 | <0.2-<0.2 | <1 | <1-<1 |
| Bacterial Endotoxins | ≤2.0 | EU/mL | 0.9 | 0.4-1.5 | <0.8 | <0.8-<0.8 |

| | | | | | |
|---|---|---|---|---|---|
| Bioburden | TAMC, ≤1 CFU/10 mL | 0 | 0-0 | <1 | <1-<1 |
| | TYMC, ≤1 CFU/10 mL | 0 | 0-0 | <1 | <1-<1 |

NA = not applicable
^{a}Mean and range for ublituximab are calculated from n = 5 batches analyzed by current version of SEC method
^{b} Two batches are pending
^{c} Controlled in-process data

### Characterization Comparison

The structural and functional activity of ublituximab high concentration (200 mg/mL) was further compared to that of ublituximab IV by characterization. A high level-summary of the structural and functional characterization results for ublituximab high concentration (200 mg/mL) is provided in Table 13. Further discussion of the comparability is provided below.

TABLE 13
Structural and Functional Characterization Results for Ublituximab High
Concentration (200 mg/mL)

| Attribute | Method | Result |
|---|---|---|
| Primary Structure | Intact Mass by LC-MS | Consistent with theoretical |
| | | molecular weights for |
| | | ublituximab |
| | Sequence Coverage and Post- | Consistent with ublituximab IV |
| | Translational Modifications | |
| | (PTMs) by Peptide | |
| | Mapping/LC-MS | |
| Higher Order Structure | Thermal Unfolding by | Differences observed in profile. |
| | Differential Scanning | Not unexpected with change in |
| | Calorimetry (DSC) | formulation. |
| | Size Distribution and | Consistent with ublituximab IV |
| | Oligomerization State by | |
| | Analytical Ultracentrifugation | |
| | (AUC) | |
| Glycosylation Profile | N-Linked Glycan Analysis | Consistent with ublituximab IV |
| Functional Activity | CD20 Cell-Based Binding | 123% potency relative to |
| | Assay | ublituximab IV reference |
| | | standard |
| | FcgRIIIa-158V Binding Assay | 121% potency relative to |
| | | ublituximab IV reference |
| | | standard |
| | C1q Binding ELISA | 95% potency relative to |
| | | ublituximab IV reference |
| | | standard |

### LC-MS Peptide Mapping Data Comparison

Primary sequence and post-translational modifications (PTMs) were characterized by LC-MS peptide mapping analysis. The primary sequence is comparable, as expected. Additionally, the relative abundance of PTMs for ublituximab high concentration (200 mg/mL) is consistent with those for ublituximab IV, as shown in Table 14.

TABLE 14
Summary of Sequence Coverage and Relative Abundance of Post-translational
Modification in Trypsin/Lys-C Peptide Mapping

| | | | Ublituximab | |
|---|---|---|---|---|
| | | | High | Ublituximab IV |
| | | | Concentration | Reference |
| | | | (200 mg/mL), | Standard STD- |
| Site | Sequence | Modification | PD Run #3 | B7A-01 |
| HC1-19 | QAYLQQSGAELVRPGASVK | Pyro-Glu | 100 | 100 |
| | (SEQ ID NO: 39) | | | |
| HC 24- | ASGYTFTSYNMHWVK | Oxidation | 0.2 | 0.2 |
| 38 | (SEQ ID NO: 18) | | | |
| HC 43- | QGLEWIGGIYPGNGDTSYNQK | Deamidated | 0.1 | 0.1 |
| 63 | (SEQ ID NO: 40) | | | |
| HC 43- | QGLEWIGGIYPGNGDTSYNQK | Oxidation | 0.1 | 0.1 |
| 63 | (SEQ ID NO: 40) | | | |
| HC 99- | YDYNYAMDYWGQGTSVTVSSASTK | Oxidation | 0.4 | 0.4 |
| 122 | (SEQ ID NO: 41) | | | |
| HC | DTLMISR | Oxidation | 3.6 | 2.6 |
| 250- | (SEQ ID NO: 42) | | | |
| 256 | | | | |
| HC | FNWYVDGVEVHNAK | Iso D | 0.3 | 0.3 |
| 276- | (SEQ ID NO: 25) | | | |
| 289 | | | | |
| HC | TKPREEQYNSTYR | Glycosylation: | 1.0 | 1.2 |
| 290- | (SEQ ID NO: 43) | G0-Gn | | |
| 302 | | | | |
| HC | TKPREEQYNSTYR | Glycosylation: | 0.3 | 0.4 |
| 290- | (SEQ ID NO: 43) | G0F-Gn | | |
| 302 | | | | |
| HC | TKPREEQYNSTYR | Glycosylation: | 58.4 | 55.4 |
| 290- | (SEQ ID NO: 43) | G0 | | |
| 302 | | | | |
| HC | TKPREEQYNSTYR | Glycosylation: | 18.6 | 21.1 |
| 290- | (SEQ ID NO: 43) | G0F | | |
| 302 | | | | |
| HC | TKPREEQYNSTYR | Glycosylation: | 11.8 | 11.8 |
| 290- | (SEQ ID NO: 43) | G1 | | |
| 302 | | | | |
| HC | TKPREEQYNSTYR | Glycosylation: | 4.2 | 4.6 |
| 290- | (SEQ ID NO: 43) | G1F | | |
| 302 | | | | |
| HC | TKPREEQYNSTYR | Glycosylation: | 0.6 | 0.6 |
| 290- | (SEQ ID NO: 43) | G2 | | |
| 302 | | | | |
| HC | TKPREEQYNSTYR | Glycosylation: | 2.2 | 2.0 |
| 290- | (SEQ ID NO: 43) | G1B | | |
| 302 | | | | |
| HC | TKPREEQYNSTYR | Glycosylation: | 0.4 | 0.5 |
| 290- | (SEQ ID NO: 43) | G2F | | |
| 302 | | | | |
| HC | TKPREEQYNSTYR | Glycosylation: | 1.6 | 1.8 |
| 290- | (SEQ ID NO: 43) | G1FB | | |
| 302 | | | | |
| HC | VVSVLTVLHQDWLNGK | Deamidated | 1.5 | 1.8 |
| 303- | (SEQ ID NO: 44) | | | |
| 318 | | | | |
| HC324- | VSNKALPAPIEK | Glycation | 0.2 | 0.2 |
| 335 | (SEQ ID NO: 26) | | | |
| HC | VSNK | Deamidated | 0.2 | 0.2 |
| 324- | (SEQ ID NO: 45) | | | |
| 327 | | | | |
| HC | NQVSLTCLVK | Deamidated | 0.4 | 0.5 |
| 362- | (SEQ ID NO: 27) | | | |
| 371 | | | | |
| HC | GFYPSDIAVEWESNGQPENNYK | Deamidated | 0.8 | 0.8 |
| 372- | (SEQ ID NO: 28) | | | |
| 393 | | | | |
| HC | WQQGNVFSCSVMHEALHNHYTQK | Deamidated | 0.4 | 0.4 |
| 418- | (SEQ ID NO: 46) | | | |
| 440 | | | | |
| HC | WQQGNVFSCSVMHEALHNHYTQK | Oxidation | 0.8 | 0.5 |
| 418- | (SEQ ID NO: 46) | | | |
| 440 | | | | |
| HC | SLSLSPGK | Cterm K | 30.4 | 34.3 |
| 441- | (SEQ ID NO: 47) | | | |
| 447 | | | | |
| LC | QIVLSQSPAILSASPGEK | Gln->pyro-Glu | 98.6 | 98.7 |
| 1-18 | (SEQ ID NO: 48) | | | |
| LC | SGTASVVCLLNNFYPR | Deamidated | 0.5 | 0.5 |
| 126- | (SEQ ID NO: 49) | | | |
| 141 | | | | |
| LC | VDNALQSGNSQESVTEQDSK | Iso D | 0.1 | 0.2 |
| 149- | (SEQ ID NO: 32) | | | |
| 168 | | | | |
| LC 77- | VEAEDAATYYCQQWTFNPPTFGGGTR | Oxidation | 2.1 | 1.5 |
| 102 | (SEQ ID NO: 50) | | | |

Note:
Site modifications shown in bold.

### Glycan Species Comparison

The relative abundance and retention time of glycan species for ublituximab high concentration (200 mg/mL) and ublituximab IV Reference Standard are shown in Table 15. The values are comparable between ublituximab high concentration (200 mg/mL) and ublituximab IV. This includes the batch release variants of total fucosylated glycan species and afucosylated glycan species, GO.

TABLE 15
Retention Time and Relative Abundance of Glycan
Species Detected in Ublituximab High Concentration
(200 mg/mL) and Ublituximab IV

| | Ublituximab High | | Ublituximab IV | |
|---|---|---|---|---|
| | Concentration | | Reference Standard | |
| | (200 mg/mL), PD Run #3 | | STD-B7A-01 | |
| | Retention | | Retention | |
| | Time | Abundance | Time | Abundance |
| Glycan Species | (min.) | (%) | (min.) | (%) |
| G0-GN | 11.7 | 0.8 | 11.7 | 0.8 |
| G0F-GN | 13.3 | 0.6 | 13.3 | 0.6 |
| G0 | 13.9 | 45.9 | 14.0 | 43.6 |
| G1-GN | 14.3 | 0.4 | 14.3 | 0.3 |
| G0B | 15.1 | 11.8 | 15.1 | 11.1 |
| G0F | 15.5 | 15.6 | 15.5 | 18.1 |
| Mannose 5 | 16.0 | 0.4 | 16.0 | 0.3 |
| G0FB | 16.6 | 6.2 | 16.6 | 7.3 |
| G1 | 16.8 | 6.5 | 16.8 | 6.3 |
| G1′ | 17.1 | 3.1 | 17.1 | 3.0 |
| G1B | 17.7 | 1.9 | 17.7 | 1.7 |
| G1F | 18.2 | 3.3 | 18.2 | 3.3 |
| G1F′ | 18.6 | 1.2 | 18.6 | 1.1 |
| G1FB | 18.9 | 1.5 | 19.0 | 1.6 |
| G2 | 19.8 | 0.5 | 19.8 | 0.5 |
| G2F | 21.1 | 0.4 | 21.1 | 0.4 |

### Higher Order Structure Comparison

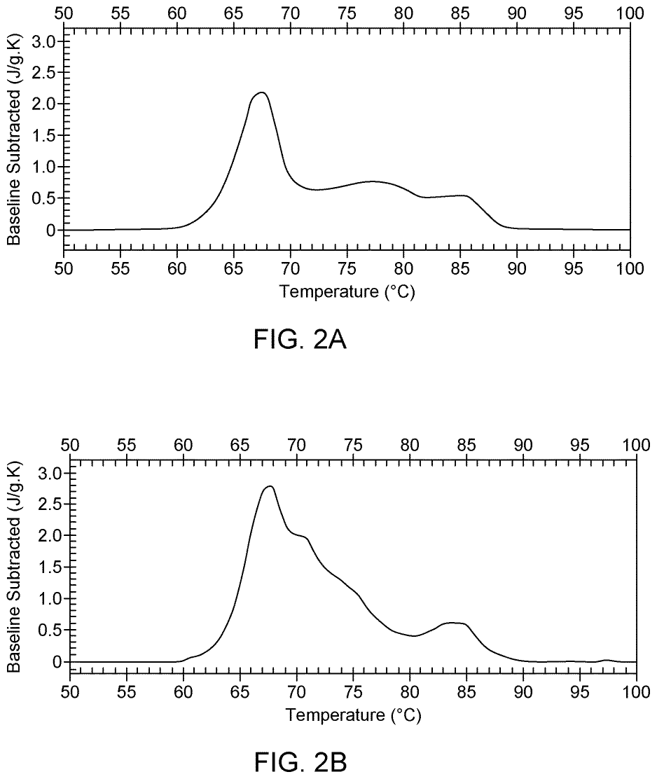

Thermal unfolding of ublituximab high concentration (200 mg/mL) was measured using differential scanning calorimetry. The DSC thermograms of ublituximab high concentration (200 mg/mL) and ublituximab IV are comparable, as shown in FIGS. 2A-2B. The deconvoluted Tₘ values for ublituximab high concentration (200 mg/mL) and ublituximab IV are comparable as shown in Table 16. There are multiple transition temperatures I detected, representing unfolding of Fab and CH domains in ublituximab.

TABLE 16
Summary of Transition Temperatures

| | Tₘ₁ | Tₘ₂ | Tₘ₃ | Tₘ₄ | Tₘ₅ |
|---|---|---|---|---|---|
| Description | (° C.) | (° C.) | (° C.) | (° C.) | (° C.) |
| Ublituximab High Concentration | 67.1 | — | 77.0 | — | 85.4 |
| (200 mg/mL) | | | | | |
| Ublituximab IV Reference Standard | 67.1 | 70.0 | 76.2 | 83.9 | — |

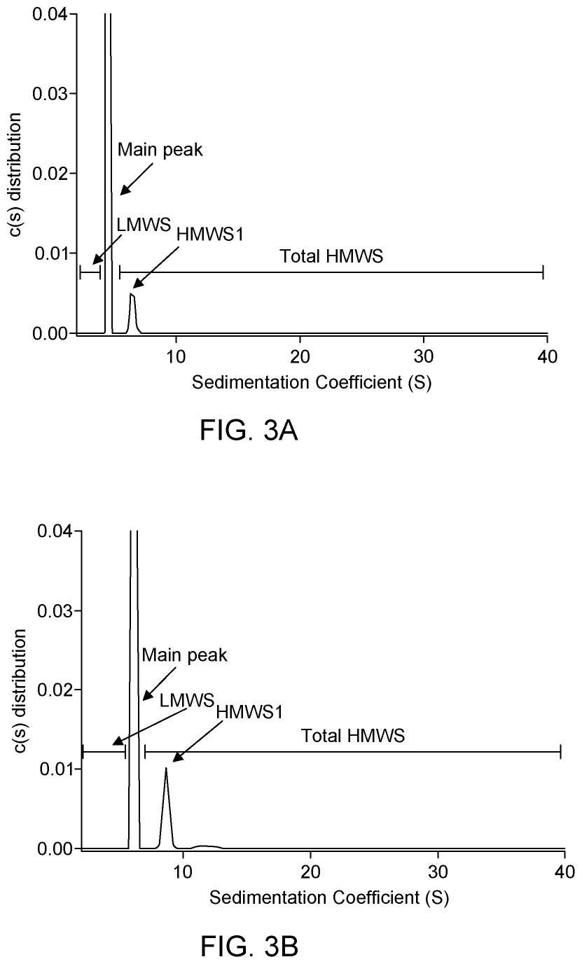

Analytical ultracentrifugation (AUC) was used as an orthogonal technique to SEC to measure size distribution and oligomerization state of ublituximab high concentration (200 mg/mL). The sedimentation coefficient distribution plots for ublituximab high concentration (200 mg/mL) and ublituximab IV are shown in FIGS. 3A-3B. The percentages of dimer and monomer are comparable for ublituximab high concentration (200 mg/mL) and ublituximab IV as summarized in Table 17 and will continue to be monitored during product development. Dimer and aggregate levels of ublituximab high concentration (200 mg/mL) are monitored through SEC acceptance criteria applied at product release.

TABLE 17
Percentage Main Peak (Monomer) and Dimer by AUC

| | Monomer | Dimer |
|---|---|---|
| Description | (%) | (%) |
| Ublituximab High Concentration (200 mg/mL) | 96.9 | 2.1 |
| Ublituximab IV Reference Standard STD-B7A-01 | 96.2 | 3.0 |

### Functional Activity Data Comparison

The functional activity of ublituximab high concentration (200 mg/mL) was compared to that of ublituximab IV by three characterization assays, namely a CD20 binding cell-based bioassay, an FcγRIIIa-158V binding assay, and a C1q ELISA. The results are shown in Table 18 and indicate that ublituximab high concentration (200 mg/mL) is functionally comparable to ublituximab IV.

TABLE 18
Functional Activity Data for Ublituximab High Concentration
(200 mg/mL) and Ublituximab IV Drug Substance Batches

| | Ublituximab High | | |
|---|---|---|---|
| | Concentration | | |
| | (200 mg/mL) | Ublituximab | |
| | (N = 1) | IV (N = 11) | |
| Test Method | Result | Average | Range |
| CD20 binding cell-based | 123% | 115% | 92%-130% |
| bioassay | | | |
| FcγRIIIa-158V binding | 121% | 115% | 106%-122% |
| assay | | | |
| C1q binding by ELISA | 95% | 101% | 88%-113% |

### Stability Data Comparison

Stability data obtained thus far for representative lots of ublituximab high concentration (200 mg/mL) drug substance and drug product are comparable to stability data obtained for ublituximab IV. The sponsor will continue to monitor product stability and its comparability to that of ublituximab IV as product development progresses.

### Conclusions

For purity, heterogeneity and functional activity attributes, ublituximab high concentration (200 mg/mL) is comparable to ublituximab IV. In addition, it shows comparable primary and higher order structure, and process- and product-related impurity profiles. Content-based attributes for ublituximab high concentration (200 mg/mL) are different from ublituximab IV including higher total protein and osmolality, and lower pH than ublituximab IV. These attributes fall within acceptable ranges and are controlled at product release. The sponsor will continue to monitor and control these attributes during product development.

### Example 3. Description and Composition of Ublituximab Solution for Injection (200 mg/ml)

Ublituximab solution for injection (200 mg/ml) is a sterile solution containing 200 mg/mL of ublituximab. The product is a single use vial designed only for subcutaneous injection.

### Composition

Each vial of drug product contains 400 mg of ublituximab in a 2 mL solution in a single-use vial. Excipient composition is based on the average of quantitation data from development and GMP drug substance batches to date. Base used for pH adjustment is also included in the composition for reference; however, amount is noted as quantum sufficit (QS), as the need for and extent of pH adjustment varies batch-to-batch. The composition is provided in Table 19.

TABLE 19
Composition of Ublituximab, Solution for Injection

| INGREDIENTS | FORMULA | | REFERENCE TO |
|---|---|---|---|
| Active substance: | per vial | FUNCTION | STANDARDS |
| Ublituximab (mg) | 400 | Active substance | Sponsor standard |
| L-Arginine (mg) | 57.8 | Buffering agent, | USP/Ph. Eur. (0806) |
| | | protein stabilization, | |
| | | tonicity modification, | |
| | | viscosity reduction | |
| L-Glutamic acid (mg) | 65.9 | Buffering agent, | USP/Ph. Eur. (0750) |
| | | stabilizer, tonicity | |
| | | modification | |
| Polysorbate 80 (mg) | 1.1 | Stabilizer | NF/Ph. Eur. (0428) |
| Sodium hydroxide, 10N | QS | pH adjustment | NF/Ph. Eur. (5110) |
| Water for injections (mL) | QS to 2 | Solvent | USP/Ph. Eur. (0169) |
| Excipient(s): | | | |
| No additional excipient is added during | | | |
| manufacture of the finished product | | | |

### Container Closure

Ublituximab solution for injection (200 mg/ml) is filled in Type I, colorless glass vials with siliconized chlorobutyl rubber stoppers that contain a barrier film and sealed with aluminum caps with a plastic button.

### Example 4. Description and Composition of Ublituximab, Injection for IV Administration

Ublituximab injection for IV administration, is a sterile solution containing 25 mg/mL of ublituximab. The product is a single use vial designed only for intravenous use after dilution in 0.9% sodium chloride.

### Composition for IV Administration

Each vial of drug product contains 150 mg/6 mL solution in a single-use vial. The composition is provided in Table 20. This composition is described in detail in WO 2023/235764, the entire contents of which are incorporated herein by reference.

TABLE 20
Composition of Ublituximab, Injection for IV Administration

| INGREDIENTS | FORMULA | | REFERENCE TO |
|---|---|---|---|
| Active substance: | per vial | FUNCTION | STANDARDS |
| Ublituximab (mg) | 150.0 | Active | Sponsor standard |
| Excipient(s): | | substance | |
| Sodium chloride (mg) | 54.0 | Tonicity agent | USP/Ph. Eur. |
| | | | (0193) |
| Sodium citrate¹ (mg) | 44.1 | Buffer and | USP/Ph. Eur. |
| | | tonicity agent | (0412) |
| Polysorbate 80 (mg) | 4.2 | Stabilizer | USP/NF/Ph. Eur. |
| | | | (0428) |
| Hydrochloric acid, | 1.2 | pH adjustment | NF/Ph. Eur. |
| concentrated² (mg) | | | (0002) |
| Water for | QS to 6.0 | Solvent | USP/Ph. Eur. |
| injections (mL) | | | (0169) |

¹Formula weight is based on dihydrate form
²Contains 2.4 mg hydrochloric acid 6M

### Container Closure

Ublituximab drug product for IV administration is filled in Type I, colorless glass vials with siliconized chlorobutyl rubber stoppers that contain a barrier film and sealed with aluminum caps with a plastic button.

### Example 5. Pharmaceutical Development of Ublituximab Solution for Injection (200 mg/mL)

### Formulation Development

A drug product formulation was developed for subcutaneous injection of ublituximab at 25 mg/mL to ensure protein structural integrity and stability. The ublituximab solution for injection (200 mg/mL) (166 mM Arginine, 224 mM Glutamate, and 0.05% polysorbate 80, pH 5.5) uses a different formulation to achieve a more highly concentrated product while avoiding injection irritating excipients such as citrate buffer and minimizing solution viscosity increases due to increased antibody concentration.

The objective of the 200 mg/mL formulation was to provide a higher product concentration more suitable for subcutaneous administration that balances the increased excipient tonicity with viscosity response minimization. Development studies included assessment of ublituximab solution for injection (200 mg/mL) quality at higher product concentrations after multiple freeze/thaw cycles, agitation assessment, and in a stressed stability study performed at higher storage temperatures. Results from these studies provide confirmation that the 200 mg/mL formulation is suitable for use at the drug substance and drug product intended storage conditions.

### Development Studies of High Concentration Clinical Formulation

Initial formulation development was performed to assess the feasibility of a higher concentration of ublituximab in an Arginine/Glutamate buffer at varying buffer composition and pH. Ublituximab was concentrated from a UFDF Diluted Pool using a Pellicon Biomax XL cartridge. In general, samples were concentrated to ~250 mg/mL for each variant and diluted as checked using Solo VPE until four target concentrations were reached (~150, 180, 225, and 250 mg/mL). Table 21 summarizes the measured concentrations, viscosity, and osmolality through the formulation preparation phases.

TABLE 21
Formulation Feasibility- Concentration, Viscosity, and Osmolality

| Formulation | | | | |
|---|---|---|---|---|
| (Nominal excipient | | [Ublituximab] | Viscosity | Osmolality |
| concentration) | Variant | (mg/mL) | (cP) | (mOsm/kg) |
| 225 mM Arg/245 | 1A | 153.9 | 8.66 | 443 |
| mM Glu, pH 5.5 | 1B | 186.5 | 15.32 | 445 |
| | 1C | 230.7 | 31.33 | 465 |
| | 1D | 262.5 | 65.18 | 471 |
| 225 mM Arg/245 | 2A | 155.1 | 7.95 | 436 |
| mM Glu, pH 6.5 | 2B | 186.5 | 15.99 | 439 |
| | 2C | 234.0 | 39.48 | 450 |
| | 2D | 254.5 | 59.68 | 456 |
| 300 mM Arg/325 | 3A | 154.2 | 8.13 | 584 |
| mM Glu, pH 5.5 | 3B | 185.1 | 15.69 | 592 |
| | 3C | 232.1 | 36.24 | 607 |
| | 3D | 264.6 | 77.96 | 614 |
| 300 mM Arg/325 | 4A | 168.5 | 10.05 | 577 |
| mM Glu, pH 6.5 | 4B | 203.7 | 21.29 | 583 |
| | 4C | 255.4 | 48.84 | 590 |
| | 4D | 284.0 | 107.40 | 600 |
| 450 mM Arg/485 | 5A | 155.8 | 9.799 | 900 |
| mM Glu, pH 5.5 | 5B | 184.0 | 17.62 | 927 |
| | 5C | 235.0 | 45.49 | 950 |
| | 5D | 272.2 | 92.05 | 976 |
| 450 mM Arg/485 | 6A | 160.1 | 11.6 | 910 |
| mM Glu, pH 6.5 | 6B | 191.0 | 26.62 | 931 |
| | 6C | 222.2 | 61.59 | 950 |
| | 6D | 255.3 | 132.4 | 992 |
| No Buffer pH 6.5 | 7A | 133.9 | 6.28 | 2 |
| | 7B | 159.9 | 12.28 | 4 |
| | 7C | 201.3 | 31.01 | 6 |
| | 7D | 218.5 | 55.48 | 27 |
| No Buffer + 150 | 8A | 128.0 | 5.268 | 283 |
| mM NaCl pH 6.5 | 8B | 150.7 | 9.46 | 290 |
| | 8C | 183.2 | 20.65 | 292 |
| | 8D | 201.9 | 30.96 | 296 |

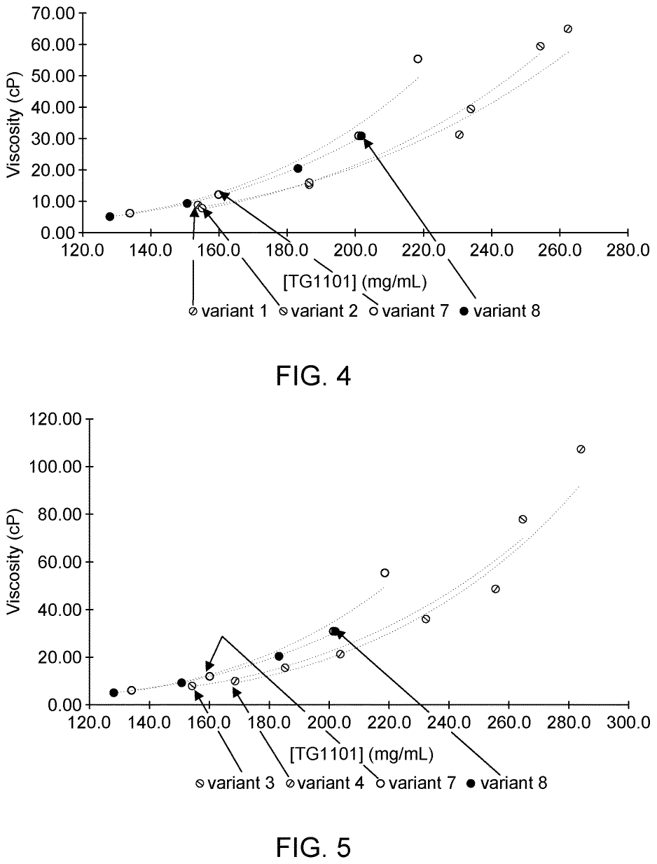

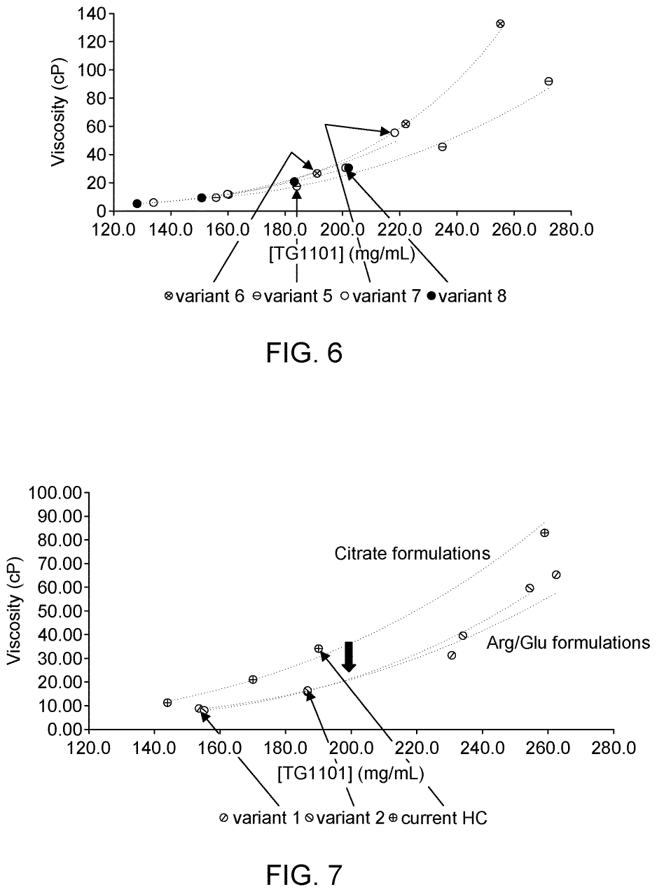

All formulations were slightly opalescent and visible particle/precipitate-free. The samples were then subjected to a viscosity and osmolality assessment. The viscosity in the formulation increases exponentially with ublituximab concentration as illustrated in FIG. 4, FIG. 5, and FIG. 6. For each buffer system/pH the viscosity response was lowered as compared to the no buffer and no buffer +150 mM NaCl controls (variants 7 and 8), with the lowest buffer system (nominally 225/245 mM Arginine/Glutamate) producing the best viscosity reduction. Osmolality was also measured via vapor pressure osmometry. Additionally, size exclusion chromatography was performed to establish an understanding of the concentration effects on the dimer species and iCIEF performed to understand effects on charge heterogeneity. There was a minimal viscosity effect across the Arginine/Glutamate buffer systems due to pH.

From this initial assessment the lower buffer formulations produced the most effective viscosity enhancements at protein concentrations above 150 mg/mL (FIG. 4).

### Excipient Concentration

One challenge associated with high-concentration formulations is increased electrostatic interaction between proteins and excipients, resulting in an offset between excipient levels in final products from ultrafiltration (UF) process buffers. The Gibbs-Donnan effect (also referred to as the Donnan effect) is the result of unequal partitioning of permeable charged species across a semi-permeable membrane due to the retention of non-permeable charged species on one side of the membrane (Kannan et al., 2023).

The diafiltration (DF) buffer used to prepare this high concentration formulation DS is composed of 225 mM Arginine and 245 mM glutamate. Subsequent quantitation of these excipients in the formulated DS (and DP given this is a ready-to-fill formulation) of development and GMP batches are captured below in Tables 22A-22C. The putative Donnan effect for this buffer system and UFDF process was demonstrated and in the expected range of DF buffer excipient reduction.

TABLE 22A
DS Excipient Quantitation

| | | Arginine | Glutamate |
|---|---|---|---|
| Sample | Type | (mM) | (mM) |
| PD Run#1 | Development | 182 | 237 |
| PD Run#2 | Development | 159 | 222 |
| PD Run#3 | Development | 162 | 218 |
| 2VAC2401CA | GMP | 164 | 225 |
| 2VAC2402CA | GMP | 163 | 225 |
| 2VAC2403CA | GMP | 166 | 219 |
| Mean ± SD | NA | 166 ± 8 | 224 ± 7 |

TABLE 22B
Concentration Ranges for Arginine (Arg) and Glutamate (Glu)

| | “Cen”ers” = Avgs | | | |
|---|---|---|---|---|
| | | | Glu (mM) | |
| | Arg (mM) | | | % of historical |
| | 153 | | 213 | batches used |
| | Range | % of historical | Range | in calc (not |
| BASIS | (mM) | batches used in | (mM) | including |
| FOR | MIN | calc that are in | MIN | outliers) that |
| RANGES | MAX | range | MAX | are in range |
| 1 SD | 140 | 12/16 = 75% | 203 | 8/11 = 73% |
| RANGE | 166 | | 223 | |
| 2 SD | 127 | 100% | 194 | 100% |
| RANGE | 179 | | 232 | |
| 3 SD | 114 | 100% | 184 | 100% |
| RANGE | 192 | | 242 | |

TABLE 22C
Concentration Ranges for Arginine (Arg) and Glutamate (glu)

| | | Arg | | Glu |
|---|---|---|---|---|
| | Arg | (mg per | Glu | (mg per |
| | (mM) | 2 mL) | (mM) | 2 mL) |
| Centers | 153 | 53.3 | 213 | 62.7 |

| | | | | | |
|---|---|---|---|---|---|
| −2 | SD | 127 | 44.2 | 194 | 57.1 |
| +2 | SD | 179 | 62.4 | 232 | 68.3 |
| −3 | SD | 114 | 39.7 | 184 | 54.1 |
| +3 | SD | 192 | 66.9 | 242 | 71.2 |

Development of the 200 mg/mL Clinical Formulation

To expand further on the studies described above, additional development work on the 200 mg/mL formulation were performed to optimize the clinical configuration for subcutaneous administration. The development studies included assessment of ublituximab product quality, after multiple freeze/thaw cycles, after 72 h agitation, and in a stressed stability study performed at higher storage temperatures. Results are discussed below.

### Freeze/Thaw Studies

In this study, formulations prepared as described above were subjected to five freeze-thaw cycles. A cycle consisted of freezing the formulation at −80° C. overnight and thawing on the benchtop at RT for at least 3 hours. Appearance, soluble aggregate by SEC, concentration by A280, charged variants by iCIEF, and subvisible particles by microflow imaging (MFI) were performed.

Physical appearance was noted for samples from vials that underwent five freeze/thaw cycles. The freeze/thaw study showed no change in the appearance of all samples as they all have a similar light-yellow color and opalescence to the t=0 samples.

The samples were analyzed using the MFI to quantitate and identify particles. Briefly, the MFI was purged with 0.19 mL followed by a 0.30 mL run. The particle count data is shown in Table 23. The particles observed have the typical morphology of protein aggregate and precipitate.

Results are shown for the selected 200 mg/mL formulation concentration at varying PS80 levels in Table 23. The results show no significant changes are observed in the product quality attributes tested, except for subvisible particles. The results for subvisible particles show differing responses with cycle number; however, all results remain within acceptable levels for release test acceptance criteria through the five freeze/thaw cycles tested. The freeze/thaw data results provided acceptable support for the 200 mg/mL formulation and long-term storage of ublituximab high concentration (200 mg/mL) at ≤−60° C.

### Agitation

Prepared formulations were placed on an orbital shaker at 500 rpm at room temperature for 72 hours. Appearance and soluble aggregates were monitored at 24, 48 and 72 hours. Each formulation underwent further analytics at the 72-hour timepoint: concentration by A280, charged variants by icIEF, and size exclusion chromatography, and subvisible particles by MFI (Table 24).

TABLE 23
Pilot Freeze/Thaw Study for Ublituximab Solution for Injection
Concentrated to 200 mg/mL at Varying PS80 Levels

| | | | | | Freeze/ | Freeze/ | Freeze/ |
|---|---|---|---|---|---|---|---|
| | | Control | Control | Control | Thaw | Thaw | Thaw |
| | | 0 F./T, | 0 F./T, | 0 F./T, | 5 cycles, | 5 cycles, | 5 cycles, |
| Test | Target | 0.0% PS80 | 0.02% PS80 | 0.05% PS80 | 0.0% PS80 | 0.02% PS80 | 0.05% PS80 |
| Concentration | 200 | 199 | 200 | 201 | 198 | 199 | 196 |
| | mg/mL | | | | | | |
| Isoform | Acidic | 28 | 29 | 29 | 28 | 29 | 27 |
| distribution | ≤32% | | | | | | |
| by iCIEF | Main | 52 | 51 | 51 | 52 | 51 | 54 |
| | ≥40% | | | | | | |
| | Basic | 20 | 20 | 20 | 20 | 20 | 19 |
| | ≤40% | | | | | | |
| SEC | Monomer | 98.1 | 98.1 | 98.2 | 98.3 | 98.1 | 98.0 |
| | ≥95.0% | | | | | | |
| | Dimer | 2.0 | 1.9 | 1.8 | 1.7 | 1.9 | 2.0 |
| | ≤5.0% | | | | | | |
| Subvisible | 25-50 μm: | 84 | 31 | 61 | 23 | 8 | 15 |
| particles | ≤600 | | | | | | |
| | particles/mL | | | | | | |
| | 10-25 μm: | 801 | 390 | 603 | 580 | 229 | 435 |
| | ≤6000 | | | | | | |
| | particles/mL | | | | | | |

TABLE 24
Pilot Agitation Study for Ublituximab Solution for Injection
Concentrated to 200 mg/mL at Varying PS80 Levels

| | | Control | Control | Control | Agitation | Agitation | Agitation |
|---|---|---|---|---|---|---|---|
| | | 0 H, | 0 H, | 0 H, | 72 H, | 72 H, | 72 H, |
| | | 0.0% | 0.02% | 0.05% | 0.0% | 0.02% | 0.05% |
| Test | Target | PS80 | PS80 | PS80 | PS80 | PS80 | PS80 |
| Concentration | 200 mg/mL | 199 | 200 | 201 | 199 | 201 | 201 |
| | Acidic | 28 | 29 | 29 | 28 | 28 | 29 |
| | ≤32% | | | | | | |
| Isoform | Main | 52 | 51 | 51 | 50 | 54 | 52 |
| distribution | ≥40% | | | | | | |
| by iCIEF | Basic | 20 | 20 | 20 | 20 | 19 | 19 |
| | ≤40% | | | | | | |
| SEC | Monomer | 98.1 | 98.1 | 98.2 | 97.9 | 98.1 | 98.1 |
| | ≥95.0% | | | | | | |
| | Dimer | 2.0 | 1.9 | 1.8 | 2.1 | 2.0 | 1.9 |
| | ≤5.0% | | | | | | |
| Subvisible | 25-50 μm: | 84 | 31 | 61 | 153 | 8 | 15 |
| particles | ≤600 | | | | | | |
| | particles/mL | | | | | | |
| | 10-25 μm: | 801 | 390 | 603 | 2221 | 351 | 366 |
| | ≤6000 | | | | | | |
| | particles/mL | | | | | | |

### Accelerated Stability Study

The ublituximab solution for injection (200 mg/mL) formulations were evaluated in a short-term stability study across differing antibody concentrations. Formulations were assessed after storage at stressed (40° C.) conditions for up to 1 month. The results are shown in Table 25.

Stability trends were more pronounced for dimer (SEC) and % acidic species (iCIEF) at the 1-month timepoint as shown in Table 25. The results are comparable to the results seen during testing of the 25 mg/mL formulation stored at 40° C. of the ublituximab IV IMPD.

The stability of ublituximab solution for injection (200 mg/mL), including real time developmental stability testing under the recommended storage conditions for both drug substance and drug product are described later.

TABLE 25
Stability Study Results at 40° C.

| | Formulation | | | | | | | | |
|---|---|---|---|---|---|---|---|---|---|
| | (Nominal | | | SEC- % | SEC- % | SEC- % | iciEF-% | iciEF-% | iciEF-% |
| | excipient | | [mAb], | Dimer | Dimer | Dimer | Acidic | Acidic | Acidic |
| Variant | concentration) | pH | mg/mL | 0 W | 2 W | 4 W | 0 W | 2 W | 4 W |
| 1a | 225 mM Arg, | 5.5 | 154 | 1.3 | 1.3 | 1.6 | 31 | 39 | 47 |
| | 245 mM Glu | | | | | | | | |
| 1b | 225 mM Arg, | 5.5 | 187 | 1.3 | 1.3 | 1.6 | 31 | 38 | 47 |
| | 245 mM Glu | | | | | | | | |
| 1c | 225 mM Arg, | 5.5 | 231 | 1.4 | 1.4 | 1.7 | 32 | 38 | 48 |
| | 245 mM Glu | | | | | | | | |
| 1d | 225 mM Arg, | 5.5 | 263 | 1.4 | 1.4 | 1.8 | 31 | 38 | 47 |
| | 245 mM Glu | | | | | | | | |
| 2a | 225 mM Arg, | 6.5 | 155 | 1.4 | 1.3 | 1.8 | 31 | 40 | 50 |
| | 245 mM Glu | | | | | | | | |
| 2b | 225 mM Arg, | 6.5 | 187 | 1.4 | 1.4 | 1.8 | 31 | 39 | 51 |
| | 245 mM Glu | | | | | | | | |
| 2c | 225 mM Arg, | 6.5 | 234 | 1.4 | 1.5 | 2.0 | 32 | 40 | 49 |
| | 245 mM Glu | | | | | | | | |
| 2d | 225 mM Arg, | 6.5 | 255 | 1.4 | 1.6 | 2.0 | 31 | 39 | 50 |
| | 245 mM Glu | | | | | | | | |

Conclusion for the Clinical Formulation (200 mg/mL)

The freeze/thaw and agitation study results support the product concentration for the drug substance and drug product formulation at 200 mg/mL. Acceptable results were observed after multiple freeze/thaw cycles and support a frozen drug substance storage condition. Stressed stability studies were conducted, and the results confirmed that the 200 mg/mL at pH 5.5 condition is suitable for use. Results from the real time stability study further support the recommended storage of the drug substance at ≤−60° C. and the drug product at 2-8° C., respectively.

### Overages

There is no overage in ublituximab solution for injection (200 mg/mL).

### Physicochemical and Biological Properties

Ublituximab solution for injection (200 mg/mL) contains ublituximab drug substance at a pH of 5.5. The selected pH condition and formulation excipients were designed to ensure the stability, quality, and purity of the antibody. The specification and test methods were selected to ensure the identity, quality, purity, biological activity, and safety of the production and consider the physiochemical and biological properties of the product. Physical properties based on the formulation are provided in Table 26.

TABLE 26
Physical properties of Ublituximab
Solution for Injection (200 mg/mL)

| Property | Target Value | |
|---|---|---|
| Specific gravity | 1.075 | |
| pH | 5.5 | |
| Osmolality | 430 | mOsm/kg |
| Viscosity (23° C.) | 20-30 | cP |

| | |
|---|---|
| Appearance | Clarity: Slightly to very |
| | opalescent ≤ Reference |
| | suspension IV |
| | Color: Slightly yellow |
| | to yellow ≤ Y3 |

### Example 6. Controls of Critical Steps and Intermediates for Ublituximab High Concentration (200 mg/mL)

Summarized here are additional process parameters controlled during the manufacture of drug substance (DS) from the intermediate including the ultrafiltration (UF), diafiltration (DF) and formulation, fill and storage steps during the ublituximab high concentration (200 mg/mL) process.

### Controls

The acceptable ranges (AR) for the in-process control parameters of the critical steps are shown in Table 27.

Bioburden and endotoxin testing are performed routinely throughout the process using qualified assays. The action limits for excipient buffers are shown in Table 28. The microbial limits for steps and limits for process intermediates are shown in Table 29 and Table 30. Bioburden sampling is conducted before 0.2 μm filtration steps (pre-filtration where possible. Bioburden and endotoxin testing are also routinely conducted to confirm membrane equilibration effluents are acceptable. Criteria are provided in Table 31 and Table 32, respectively.

TABLE 27
In-Process Controls for Critical Steps

| Critical Step | Parameter | AR |
|---|---|---|
| Ultrafiltration and | Concentration after recovery from | ≥210 |
| Diafiltration (UFDF) | the TFF system, mg/mL | |
| Formulation | Formulation stabilization buffer | 0.05 |
| | addition, L buffer/L product pool | |
| | Formulated bulk (pre-filter) drug | −80-220 |
| | substance concentration, mg/mL | |

TABLE 28
Bioburden and Endotoxin Controls for Excipient Buffers

| Composition | Attribute | Action Limit |
|---|---|---|
| 225 mM Arginine, 245 mM Glutamate, | Bioburden | Report Results |
| pH 5.5 | Endotoxin | Report Results |
| 225 mM Arginine, 245 mM Glutamate, | Bioburden | Report Results |
| 1% Polysorbate 80, pH 5.5 | Endotoxin | Report Results |
| 225 mM Arginine, 245 mM Glutamate, | Bioburden | Report Results |
| 0.05% Polysorbate 80, pH 5.5 | Endotoxin | Report Results |

TABLE 29
Bioburden Controls from Process Intermediates

| | Bioburden, CFU/10 mL, |
|---|---|
| Process Intermediate | Action Limit |
| UFDF load (Thawed diluted | Report Results |
| UFDF pool intermediate) | |
| Formulated bulk drug | Report Results |
| substance pool (pre-filtration) | |

TABLE 30
Endotoxin Controls from Process Intermediates

| | Endotoxin, EU/mL, |
|---|---|
| Process Intermediate | Action Limit |
| UFDF load (Thawed diluted | Report Results |
| UFDF pool intermediate) | |
| Formulated bulk drug | Report Results |
| substance pool (pre-filtration) | |

TABLE 31
Bioburden Controls from Membrane Equilibration

| | Bioburden, CFU/10 mL, |
|---|---|
| Process Intermediate | Action Limit |
| UFDF membrane equilibration effluent | Report Results |

TABLE 32
Endotoxin Controls from Membrane Equilibration

| | Endotoxin, EU/mL, |
|---|---|
| Process Intermediate | Action Limit |
| UFDF membrane equilibration effluent | Report Results |

### Example 7. Specification for Ublituximab High Concentration (200 mg/mL)

Described herein are analytical test procedures used to test the specification of ublituximab high concentration (200 mg/mL). Because the active pharmaceutical ingredient in ublituximab high concentration (200 mg/mL) is the same as in ublituximab injection for IV administration, the methods between the two are essentially the same with the exception of additional predilution to achieve the correct concentrations for the methods. In addition, supplemental validation was performed for certain methods to demonstrate that the change in formulation did not impact assay performance.

### Appearance

Opalescence and color of ublituximab high concentration (200 mg/mL) were tested according to a method compliant with Ph. Eur. 2.2.1 for opalescence and Ph. Eur. 2.2.2 for color.

### Protein Concentration

Protein concentration of ublituximab high concentration (200 mg/mL) was determined using absorbance at 280 nm measured by the Solo VPE method or by UV Spectroscopy as described below.

### Protein Concentration by Solo VPE

Protein concentration of ublituximab high concentration (200 mg/mL) drug substance and drug product was measured by SoloVPE.

In the SoloVPE method, the pathlength was varied to collect multiple absorbance readings at 280 nm. Where needed, samples were diluted into the linear range of the method. The slope of the sample absorbance with pathlength was calculated; sample concentration was then determined based on the Beer-Lambert law and division of the slope of the absorbance by the extinction coefficient. The protein concentration value was reported as the mean of the triplicate determinations in mg/mL to one decimal place.

TABLE 33
Protein Concentration by Solo VPE System Suitability

| System Suitability Type | Suitability Criteria |
|---|---|
| Scan of Bovine Serum | R² value ≥0.999 for each replicate |
| Albumin Control | |
| RSD for triplicate Bovine | RSD ≤3.0% |
| Serum Albumin Control | |
| concentration measurement | |
| Bovine Serum Albumin Control | BSA concentration must be within |
| Concentration | the qualified range of the |
| | COA value |
| Bovine Serum Albumin Control | Both pre-read and post-read |
| | measurements must pass |
| | suitability criteria |
| Scan of sample | R² value ≥0.999 for each replicate |
| RSD for triplicate protein | RSD ≤3.0% |
| concentration measurement | |

RSD = relative standard deviation

### Protein Concentration by UV Spectroscopy

Protein concentration of ublituximab high concentration (200 mg/mL) drug substance was measured using UV spectroscopy at 280 nm with a light scattering correction at 320 nm. The concentration was calculated based on the Beer-Lambert law. The extinction coefficient of ublituximab high concentration (200 mg/mL) at 280 nm was 1.61 mL*mg-1*cm-1 as determined by the amino acid composition and used for all ublituximab high concentration (200 mg/mL) manufactured. Samples were diluted gravimetrically with 0.9% sodium chloride to a target protein concentration of 0.5 mg/mL. Samples were loaded into a quartz cuvette with a 1 cm pathlength and the absorbances at 280 and 320 nm were measured for protein concentration calculation. Individual measurements of ublituximab high concentration (200 mg/mL) were recorded to three decimal places. Protein concentration was reported as the mean of the triplicate determinations in mg/mL to one decimal place.

TABLE 34
Protein Concentration by UV Spectroscopy System Suitability

| | |
|---|---|
| System Suitability Type | Suitability Criteria |

| Blank absorbance | 0.000 ± 0.015 | AU |
|---|---|---|
| Bovine Serum Albumin | BSA concentration must be within | |
| Concentration | 0.08 mg/mL of the COA value | |
| Sample absorbance | 0.4-1.3 | AU |

| | |
|---|---|
| CV for triplicate protein | RSD ≤2.0% |
| concentration measurement | |

AU = absorbance unit;
RSD = relative standard deviation

### Peptide Mapping

The Peptide Mapping test procedure for ublituximab high concentration (200 mg/mL) was unchanged from that of ublituximab IV.

pH

pH was tested according to USP <791> and Ph. Eur. 2.2.3.

### Osmolality

The osmolality was measured using vapor pressure depression due to the high concentration of material inhibiting effective freezing for freezing point depression measurements.

Vapor osmometer testing is a passive technique based upon the measurement of vapor pressure depression made by thermocouple hygrometry. The thermocouple hygrometer sensor operates using a thermal energy balancing principle to measure the dew point temperature depression within the chamber. For standards and samples, 10 μL of material are reverse-pipetted on to the sample disc loaded into the instrument. Table 35 captures the system suitability acceptance criteria prior sample analyses.

All individual standard and sample results for system and assay suitability were recorded.

TABLE 35
Vapor Pressure Osmolality System Suitability

| Standard | Acceptance Criteria | |
|---|---|---|
| (mOsm/kg | (mOsm/kg) | |
| 290 | ±6 units | (284-296) |
| 1000 | ±10 units | (990-1010) |
| 100 | ±4 units | (96-104) |
| 400 | ±4 units | (396-404) |

### Size Exclusion Chromatography

The Size Exclusion Chromatography (SEC) method was used to determine the molecular size distribution and purity of ublituximab high concentration (200 mg/mL). The method was used for batch release and stability testing for ublituximab high concentration (200 mg/mL) drug substance and drug product. The method has been shown to be stability-indicating using thermally degraded samples.

Ublituximab high concentration (200 mg/mL) samples were diluted with formulation buffer initially from 200 mg/mL to 25 mg/mL. The diluted 25 mg/mL high concentration samples and the ublituximab IV reference standard were diluted to a final protein concentration of 1 mg/mL with the mobile phase, 0.3 M sodium phosphate pH 7.2. Test samples were separated on a TSKgel G3000SWXL column (Tosoh) or equivalent. The run time was 40 minutes at a flow rate of 0.5 mL/minute and column temperature of 25±3° C. The peaks were monitored at 214 nm. The peaks on the chromatogram were integrated and the amount of each peak (monomer, dimer, aggregate, and fragments) was expressed as the percentage of its area. Blanks, samples, reference standard, and molecular weight standards injections were performed to assess system suitability.

TABLE 36
Size Exclusion Chromatography System Suitability

| System Suitability | |
|---|---|
| Type | Suitability Criteria |
| Blank | Must have a stable baseline with no |
| | interfering peaks |
| Molecular Weight | Chromatograms must be comparable to |
| Standards (MWS) | example in test protocol |
| | Relative resolution of gamma globulin and |
| | ovalbumin must be ≥1.2 |
| | MWS the plate count of vitamin B12 peak must |
| | be ≥10000 |
| Reference Standard | Reference standard chromatograms must be |
| (initial 5 injections) | comparable to example in test protocol % RSD |
| | for retention time of monomer peak must |
| | be ≤2% % RSD for monomer peak area |
| | must be ≤2% |
| | Relative resolution between dimer and |
| | monomer must be ≥2.0 |
| Reference Standard | All injections must be comparable to the initial |
| (Bracketing | five suitability injections. % RSD for |
| injections) | retention time of the Monomer peak must |
| | be ≤3%. % RSD |
| | for Monomer peak area must be ≤3%. |
| Sample | % difference between monomer peak areas of |
| | duplicate injections must be ≤5% |

RSD: Relative Standard Deviation

### Capillary Gel Electrophoresis

The Capillary Gel Electrophoresis (CGE) method is a quantitative test method for monitoring product purity as well as product-related impurities. The method was performed with both reduced and non-reduced sample preparation conditions. The method was used for batch release and stability testing for ublituximab high concentration (200 mg/mL) drug substance and drug product. The method has been shown to be stability indicating using thermally degraded samples.

### CGE Reduced Method

The reduced method quantitated the light chain (LC), heavy chain (HC) and the summation of LC and HC.

Test samples were prepared by an initial dilution of ublituximab high concentration (200 mg/mL) samples with formulation buffer from 200 mg/mL to 25 mg/mL and then with SDS sample buffer from 25 mg/mL to 5 mg/mL. Samples (including test samples and reference standard) were prepared by dilution in sample buffer (SDS-MW sample buffer, 100 mM Tris-HCl, pH 9.0, 1% SDS) to a final concentration of 1 mg/mL. A 10 kDa internal standard was added to the reference standard. Samples were reduced by the addition of 2-mercaptoethanol (BME) followed by incubation at 70±2° C. for 8 to 12 minutes. The method was conducted using a bare fused silica capillary and a Beckman PA800 plus system or equivalent. Detection was by a photodiode array (PDA) detector at 220 nm wavelength. The samples were separated under 15.0 kV for 24 minutes. All samples and reference standard were tested in duplicate.

TABLE 37
CGE Reduced Method System Suitability

| System Suitability | |
|---|---|
| Type | Suitability Criteria |
| Reference | Each electropherogram must be comparable |
| Standard | to the provided electropherogram in the |
| | test method |
| | CPA for the Light Chain in each injection |
| | must be ≥3000. |
| | CPA for the Heavy Chain in each injection |
| | must be ≥6000. |
| | The % difference for the purity of the |
| | duplicate injections must be ≤2% |
| | Heavy chain % CPA must be within 60.0% |
| | to 66.3% |
| | Light chain % CPA must be within 32.8% |
| | to 37.7% |
| | Sum of Light chain and Heavy chain % |
| | CPA must be within 95.9% to 100% |
| Sample | Visual confirmation of the heavy and light |
| | chain peaks must be established in both |
| | sample injections |
| | CPA for the Light Chain in each injection |
| | must be ≥3000.CPA for the Heavy Chain |
| | in each injection must be ≥6000. |
| | The % difference for the purity of the |
| | duplicate injections must be ≤1% |

CPA: Corrected Peak Area

### CGE Non-Reduced Method

The non-reducing method of CGE measures IgG, free light chain (LC) and high molecular weight species (HMWS).

Test samples were prepared by an initial dilution of ublituximab high concentration (200 mg/mL) samples with formulation buffer from 200 mg/mL to 25 mg/mL and then with SDS sample buffer from 25 mg/mL to 5 mg/mL. Samples (including test samples and reference standard) were prepared by dilution in sample buffer (SDS-MW sample buffer, 100 mM Tris-HCl, pH 9.0, 1% SDS) to a final concentration of 1 mg/mL. A 10 kDa internal standard was added to the reference standard. Samples were alkylated by the addition of iodoacetamide followed by incubation at 63±2° C. The separation was conducted with the same equipment and method as described for the CGE reduced method above.

TABLE 38
CGE Non-reduced Method System Suitability

| System Suitability | |
|---|---|
| Type | Suitability Criteria |
| Reference | Each electropherogram must be comparable |
| Standard | to the provided electropherogram in the |
| | test method |
| | The % difference for the purity of the |
| | duplicate injections must be ≤3% |
| | IgG % CPA must be within 91.1% to 97.1% |
| | Light chain % CPA must be ≤1.9% |
| | High Molecular Species (HMWS) |
| | must be ≤1.5% |
| | Total corrected area for each reference |
| | material injection must be ≥10,000 |
| Sample | Visual confirmation of the heavy and light |
| | chain peaks must be established in both |
| | sample injections |
| | CPA for the Light Chain in each injection |
| | must be ≥3000. |
| | CPA for the Heavy Chain in each injection |
| | must be ≥6000. |
| | The % difference for the purity of the |
| | duplicate injections must be ≤1% |

### Imaged Capillary Isoelectric Focusing

The Imaged Capillary Isoelectric Focusing (iCIEF) method was performed for the determination of the distribution of charge-based variants and for the identification of ublituximab high concentration (200 mg/mL). The method was performed for drug substance and drug product batch release and stability.

Ublituximab high concentration (200 mg/mL) samples were diluted with ublituximab IV (25 mg/mL) formulation buffer initially from 200 mg/mL to 25 mg/mL. Test samples were prepared by using the diluted 25 mg/mL high concentration samples with the test sample solution of 0.15% methylcellulose, 1.2 M urea, 8% ampholytes pH 3 to 10.5, and pH 10.10 and 8.40 pI markers. Method parameters included focusing for 1 minute at 1500 V followed by 8 min at 3000 V. A blank sample and the ublituximab IV (25 mg/mL) reference standard were also run to evaluate system suitability.

The distribution of charge-based variants by iCIEF was determined by the results of the pI of the main peak, the % peak area of the main peak, pooled % peak area for the basic peaks and acidic peaks are determined. Identification of ublituximab high concentration (200 mg/mL) by iCIEF was confirmed with comparable profile to that of the reference standard.

### ADCC Potency Assay

The ADCC potency assay for ublituximab high concentration (200 mg/mL) was unchanged from that of ublituximab IV except for additional predilution steps.

### CDC Activity by Cell-Based Bioassay

The CDC potency assay for ublituximab high concentration (200 mg/mL) was unchanged from that of ublituximab IV (25 mg/mL) except for additional predilution steps.

### Host Cell Protein by ELISA

The Host Cell Protein by ELISA test procedure was used for measuring in-process impurities of the drug substance intermediate (diluted UFDF pool). The UFDF pool was similar in concentration and buffer composition to ublituximab IV (25 mg/mL), therefore the test procedure was unchanged from that of ublituximab IV (25 mg/mL).

### Residual Protein A

The Residual Protein A test procedure was used for measuring in-process impurities of the drug substance intermediate (diluted UFDF pool). The UFDF pool was similar in concentration and buffer composition to ublituximab IV (25 mg/mL), therefore the test procedure was unchanged from that of ublituximab IV (25 mg/mL).

### Residual DNA

The Residual DNA test procedure was used for measuring in-process impurities of the drug substance intermediate (diluted UFDF pool). The UFDF pool was similar in concentration and buffer composition to ublituximab IV (25 mg/mL), therefore the test procedure was unchanged from that of ublituximab IV (25 mg/mL).

### Bacterial Endotoxins

Bacterial endotoxin was tested according to a method compliant with USP <85> and Ph. Eur. 2.6.14.

The bacterial endotoxin test procedure for ublituximab high concentration (200 mg/mL) was the same as that used for ublituximab IV (25 mg/mL), except that a 1:100 dilution was performed for the high concentration formulation, compared to a 1:20 for the IV formulation, and the LAL reagent which was used in the method for from that of ublituximab IV (25 mg/mL) except for a 1:100 dilution being performed for ublituximab high concentration (200 mg/mL) rather than a 1:20 dilution that is performed for ubilituximab IV (25 mg/mL).

### Bioburden

Bioburden was tested according to a method compliant with USP <61> and Ph. Eur. 2.6.12. The bioburden test procedure for ublituximab high concentration (200 mg/mL) was the same as that used for ublituximab IV (25 mg/mL) using a 10 mL volume.

The specification for ublituximab high concentration (200 mg/mL) is provided in Table 39 below.

TABLE 39
Specification for Ublituximab High Concentration (200 mg/mL) Drug Substance

| | | Analytical | | |
|---|---|---|---|---|
| Attribute | Test | Procedure | Acceptance Criteria | |
| Appearance | Color | Ph. Eur. 2.2.2 | Slightly yellow to yellow (≤Y3) | |
| | Opalescence | USP <855> | Slightly to very opalescent | |
| | | Ph. Eur. 2.2.1 | (≤Reference suspension IV) | |
| Protein Content | Protein Concentration | ATM-1019 | 180-220 | mg/mL |
| Identity | Peptide | ATM-1021 | Complies with reference | |
| | Mapping | | standard | |
| General | pH | USP <791> | 5.2-5.7 | |
| | | Ph. Eur. 2.2.3 | | |
| | Osmolality | ATM-1025 | 400-500 | mOsm/kg |
| Purity | Size Exclusion | ATM-1010 | Monomer-≥95.0% | |
| | Chromatography | | Total Aggregates (Dimer + | |
| | | | Aggregates)- ≤5.0% | |
| | | | Fragments- ≤1.6% | |
| | Capillary Gel | ATM-1009 | ≥94.0% HC + LC | |
| | Electrophoresis | | | |
| | (Reduced) | | | |
| | Capillary Gel | ATM-1009 | ≥85.0% IgG ≤3.0% | |
| | Electrophoresis | | HMWS ≤4.0% | |
| | (Non-Reduced) | | Light Chain | |
| Heterogeneity | Imaged Capillary | ATM-1011 | pI Main: −.3-9.5 % Acidic | |
| | Isoelectric Focusing | | Peaks: ≤32.0% % Main | |
| | | | Peak: >40.0% % Basic | |
| | | | Peaks: ≤40.0% | |
| Potency | ADCC Potency Assay | ATM-1005 | 63-177% | |
| | CDC Activity by | ATM-1002 | 74-127% | |
| | Cell-based Bioassay | | | |
| Impurities^{a} | Host Cell Protein by | ATM-1014 | ≤20 | ng/mg protein |
| | ELISA | | | |
| | Residual Protein A | ATM-1022 | ≤5 | ng/mg protein |
| | Residual DNA | ATM-1013 | ≤5 | pg/mg protein |
| Safety | Bacterial Endotoxins | USP <85>; | ≤2.0 | EU/mL |
| | | Ph. Eur. 2.6.14 | | |

| | | |
|---|---|---|
| Bioburden | USP <61>; | TAMC: ≤1 CFU/10 mL |
| | Ph. Eur. 2.6.12 | TYMC: ≤1 CFU/10 mL |

Ph. Eur. = European Pharmacopeia;
USP = United States Pharmacopeia;
mOsm = milliosmoles;
EU = endotoxin units;
HMWS = high molecular weight species;
pI = isoelectric point;
ADCC = antibody dependent cellular cytotoxicity;
CDC = complement dependent cytotoxicity;
N/A = Not Applicable

### Example 8. Batch Analysis for Ublituximab High Concentration (200 mg/mL)

Batch analysis results for drug substance batches (2VAC2401CA, 2VAC2402CA, 2VAC2403CA, 2VAC2404CA, 3VAC2501CA, and 3VAC2502CA) manufactured are provided in Table 40. Batch analysis results for drug substance batches (2VAC2503CA, 2VAC2504CA, 2VAC2505CA, 2VAC2506CA, 2VAC2507CA, 2VAC2508CA, and 3VAC2509CA) manufactured are provided in Table 41. Batch analysis results for drug substance batches (656A25-01, 656A25-02, 656A25-03, and 656A25-04) manufactured are provided in Table 42. All batches met their batch release acceptance criteria.

TABLE 40
Drug Substance Batch Analysis for Batches Manufactured at Process X

| | | Batch Reference/Batch Number | | | | | |
|---|---|---|---|---|---|---|---|
| Date of Manufacture | | 2VAC2401CA | 2VAC2402CA | 2VAC2403CA | 2VAC2404CA | 3VAC2501CA | 3VAC2502CA |
| | Acceptance | 29 JUN. | 2 JUL. | 9 SEP. | 21 NOV. | 8 APR. | 19 APR. |
| Test Method | Criteria | 2024 | 2024 | 2024 | 2024 | 2025 | 2025 |
| Opalescence, | Slightly | =Reference | =Reference | =Reference | =Reference | =Reference | =Reference |
| Ph. Eur. | Opalescent to | Suspension I | Suspension I | Suspension II | Suspension II | Suspension II | Suspension I |
| 2.2.1 | Very | | | | | | |
| | Opalescent | | | | | | |
| | (≤Reference | | | | | | |
| | suspension | | | | | | |
| | IV) | | | | | | |
| Color, | Slightly | =Y4 | =Y4 | =Y4 | =Y4 | <Y3 | =Y4 |
| Ph. Eur. | Yellow | | | | | | |
| 2.2.2 | to | | | | | | |
| | Yellow | | | | | | |
| | (≤Y3) | | | | | | |
| Peptide | Complies | Complies | Complies | Complies | Complies | Complies | Complies |
| Mapping or | with | with | with | with | with | with | with |
| iCIEF | reference | reference | reference | reference | reference | reference | reference |
| | standard | standard | standard | standard | standard | standard | standard |
| pH, | 5.2-5.7 | 5.4 | 5.4 | 5.4 | 5.6 | 5.5 | 5.5 |
| Ph. Eur. | | | | | | | |
| 2.2.3 | | | | | | | |
| Osmolality | 400-500 | 429 | 429 | 432 | 436 | 419 | 429 |
| | mOsm/kg | | | | | | |
| Imaged | pI, | 9.3 | 9.3 | 9.3 | 9.3 | 9.3 | 9.3 |
| Capillary | 9.3-9.5 | | | | | | |
| Isoelectric | % Acidic | 26.3 | 26.0 | 24.5 | 25.2 | 26.4 | 26.9 |
| Focusing | Peaks, | | | | | | |
| | ≤32.0% | | | | | | |
| | % Main | 49.2 | 49.2 | 53.4 | 53.1 | 53.4 | 52.9 |
| | Peak, | | | | | | |
| | ≥40.0% | | | | | | |
| | % Basic | 24.5 | 24.8 | 22.1 | 21.7 | 20.2 | 20.3 |
| | Peaks, | | | | | | |
| | ≤40.0% | | | | | | |
| cGE - | Purity, | 97.9 | 98.0 | 97.9 | 97.3 | 98.3 | 98.2 |
| Reduced | ≥94.0% | | | | | | |
| | HC + LC | | | | | | |
| cGE - | Purity, | 93.1 | 92.8 | 93.1 | 95.3 | 94.3 | 93.9 |
| Non- | ≥85.0% | | | | | | |
| reduced | IgG | | | | | | |
| | ≤4.0% | 1.2 | 1.3 | 1.3 | 0.8 | 1.1 | 1.2 |
| | Light | | | | | | |
| | Chain | | | | | | |
| | ≤3.0% | 0.3 | 0.4 | 0.4 | 0.7 | 0.4 | 0.4 |
| | HMWS | | | | | | |
| Size | Monomer, | 97.1 | 98.0 | 98.0 | 97.8 | 98.1 | 98.1 |
| Exclusion | ≥95.0% | | | | | | |
| Chromatography | Total | 2.9 | 2.0 | 2.0 | 2.1 | 1.8 | 1.9 |
| | Aggregates | | | | | | |
| | (Dimer + | | | | | | |
| | Aggregates), | | | | | | |
| | ≤5.0% | | | | | | |
| | Fragments, | 0.1 | 0.1 | 0.1 | 0.1 | 0.1 | 0.1 |
| | ≤1.6% | | | | | | |
| Residual | ≤5 | <0.2 | <0.2 | <0.2 | <0.2 | 0 | 0 |
| Protein A^{a} | ng/mg | | | | | | |
| | protein | | | | | | |
| Host Cell | ≤20 | 5 | 6 | 5 | 7 | 5 | 5 |
| Protein by | ng/mg | | | | | | |
| ELISA^{a} | protein | | | | | | |
| Residual | ≤5 | <0.2 | <0.2 | <0.2 | <0.4 | <0.08 | <0.08 |
| DNA^{a} | pg/mg | | | | | | |
| | protein | | | | | | |
| Bacterial | ≤2.0 | 0.4 | 1.5 | 0.9 | 0.2 | 0.3 | <0.2 |
| Endotoxins, | EU/mL | | | | | | |
| Ph. Eur. | | | | | | | |
| 2.6.14 | | | | | | | |
| Bioburden, | TAMC: ≤1 | 0 | 0 | 0 | 0 | 0 | 0 |
| Ph. Eur. | CFU/10 | | | | | | |
| 2.6.12 | mL | | | | | | |
| | TYMC: ≤1 | 0 | 0 | 0 | 0 | 0 | 0 |
| | CFU/10 | | | | | | |
| | mL | | | | | | |
| Protein | 180-220 | 211 | 202 | 203 | 203 | 199 | 208 |
| Concentration | mg/mL | | | | | | |
| CDC | 74- | 93 | 92 | 81 | 97 | 104 | 111 |
| Activity by | 127% | | | | | | |
| Cell-Based | | | | | | | |
| Bioassay | | | | | | | |
| ADCC | 63- | 86 | 84 | 96 | 116 | 109 | 85 |
| Potency | 177% | | | | | | |
| Assay | | | | | | | |

^{a}Controlled in-process data
cGE, Capillary Gel Electrophoresis;
LC, light chain;
HMWS, high molecular weight species;
HCP, Host cell protein

TABLE 41
Drug Substance Batch Analysis for Batches Manufactured at Site X

| | | Batch Reference/Batch Number | | | | | | |
|---|---|---|---|---|---|---|---|---|
| Date of Manufacture | | 2VAC2503CA | 2VAC2504CA | 2VAC2505CA | 2VAC2506CA | 2VAC2507CA | 2VAC2508CA | 3VAC2509CA |
| Test | Acceptance | 14 JUN. | 25 JUN. | 27 JUN. | 26 JUL. | 28 AUG. | 2 SEP. | 19 SEP. |
| Method | Criteria | 2025 | 2025 | 2025 | 2025 | 2025 | 2025 | 2025 |
| Opalescence, | Slightly | =Reference | =Reference | =Reference | =Reference | =Reference | =Reference | =Reference |
| Ph. Eur. | Opalescent | Suspension II | Suspension II | Suspension II | Suspension II | Suspension II | Suspension II | Suspension II |
| 2.2.1 | to Very | | | | | | | |
| | Opalescent | | | | | | | |
| | (≤Reference | | | | | | | |
| | suspension | | | | | | | |
| | IV) | | | | | | | |
| Color, | Slightly | =Y4 | =Y4 | =Y4 | =Y4 | =Y4 | =Y4 | =Y4 |
| Ph. Eur. | Yellow | | | | | | | |
| 2.2.2 | to | | | | | | | |
| | Yellow | | | | | | | |
| | (≤Y3) | | | | | | | |
| Peptide | Complies | Complies | Complies | Complies | Complies | Complies | Complies | Complies |
| Mapping | with | with | with | with | with | with | with | with |
| or iCIEF | reference | Reference | Reference | Reference | Reference | Reference | Reference | Reference |
| | standard | Standard | Standard | Standard | Standard | Standard | Standard | Standard |
| pH, | 5.2- | 5.5 | 5.4 | 5.4 | 5.5 | 5.5 | 5.5 | 5.6 |
| Ph. Eur. | 5.7 | | | | | | | |
| 2.2.3 | | | | | | | | |
| Osmolality | 400- | 425 | 430 | 427 | 430 | 432 | 435 | 425 |
| | 500 | | | | | | | |
| | mOsm/kg | | | | | | | |
| Imaged | pI, | 9.3 | 9.3 | 9.3 | 9.3 | 9.3 | 9.3 | 9.3 |
| Capillary | 9.3-9.5 | | | | | | | |
| Isoelectric | % Acidic | 26.6 | 26.5 | 27.1 | 25.9 | 27.8 | 28.4 | 28.6 |
| Focusing | Peaks, | | | | | | | |
| | ≤32.0% | | | | | | | |
| | % Main | 53.0 | 53.6 | 52.9 | 53.9 | 48.0 | 47.3 | 48.1 |
| | Peak, | | | | | | | |
| | ≥40.0% | | | | | | | |
| | % Basic | 20.4 | 19.9 | 20.0 | 20.2 | 24.2 | 24.3 | 23.3 |
| | Peaks, | | | | | | | |
| | ≤40.0% | | | | | | | |
| cGE - | Purity, | 97.8 | 98.0 | 97.8 | 98.1 | 98.1 | 98.0 | 98.1 |
| Reduced | ≥94.0% | | | | | | | |
| | HC + LC | | | | | | | |
| cGE - | Purity, | 96.0 | 95.9 | 95.7 | 96.5 | 95.3 | 95.2 | 94.6 |
| Non- | ≥85.0% | | | | | | | |
| reduced | IgC | | | | | | | |
| | ≤4.0% | 0.8 | 0.8 | 0.9 | 0.6 | 0.7 | 0.8 | 0.9 |
| | Light | | | | | | | |
| | Chain | | | | | | | |
| | ≤3.0% | 0.2 | 0.2 | 0.2 | 0.2 | 0.4 | 0.5 | 0.3 |
| | HMWS | | | | | | | |
| Size | Monomer, | 98.1 | 98.1 | 98.1 | 98.2 | 97.9 | 97.9 | 97.8 |
| Exclusion | ≥95.0% | | | | | | | |
| Chromatography | Total | 1.8 | 1.8 | 1.8 | 1.7 | 2.0 | 2.0 | 2.1 |
| | Aggregates | | | | | | | |
| | (Dimer + | | | | | | | |
| | Aggregates), | | | | | | | |
| | ≤5.0% | | | | | | | |
| | Fragments, | 0.1 | 0.1 | 0.1 | 0.1 | 0.1 | 0.1 | 0.1 |
| | ≤1.6% | | | | | | | |
| Residual | ≤5 | 0 | 0 | 0 | 0 | 0 | 0 | 0 |
| Protein | ng/mg | | | | | | | |
| A^{a} | protein | | | | | | | |
| Host Cell | ≤20 | 5 | 5 | 5 | 5 | 4 | 4 | 6 |
| Protein by | ng/mg | | | | | | | |
| ELISA^{a} | protein | | | | | | | |
| Residual | ≤5 | <0.08 | <0.08 | <0.08 | <0.08 | <0.08 | <0.08 | <0.08 |
| DNA^{a} | pg/mg | | | | | | | |
| | protein | | | | | | | |
| Bacterial | ≤2.0 | <0.2 | <0.2 | <0.2 | 0.2 | 1.3 | 1.0 | 0.7 |
| Endotoxins, | EU/mL | | | | | | | |
| Ph. Eur. | | | | | | | | |
| 2.6.14 | | | | | | | | |
| Bioburden, | TAMC: ≤1 | 0 | 0 | 0 | 0 | 0 | 0 | 0 |
| Ph. Eur. | CFU/ | | | | | | | |
| 2.6.12 | 10 mL | | | | | | | |
| | TYMC: ≤1 | 0 | 0 | 0 | 0 | 0 | 0 | 0 |
| | CFU/ | | | | | | | |
| | 10 mL | | | | | | | |
| Protein | 180-220 | 210 | 200 | 203 | 210 | 203 | 197 | 212 |
| Concentration | mg/mL | | | | | | | |
| CDC | 74- | 82 | 82 | 87 | 85 | 89 | 102 | 98 |
| Activity | 127% | | | | | | | |
| by Cell- | | | | | | | | |
| Based | | | | | | | | |
| Bioassay | | | | | | | | |
| ADCC | 63- | 98 | 95 | 86 | 92 | 103 | 105 | 88 |
| Potency | 177% | | | | | | | |
| Assay | | | | | | | | |

^{a}Controlled in-process data
cGE, Capillary Gel Electrophoresis;
LC, light chain;
HMWS, high molecular weight species;
HCP, Host cell protein

TABLE 42
Drug Substance Batch Analysis for Batches Manufactured at Site Y

| | | Batch Reference/Batch Number | | | |
|---|---|---|---|---|---|
| | | 656A25-01 | 656A25-02 | 656A25-03 | 656A25-04 |
| | Acceptance | Date of Manufacture | | | |
| Test Method | Criteria | 26 JUL. 2025 | 22 SEP. 2025 | 29 SEP. 2025 | 3 OCT. 2025 |
| Opalescence, | Slightly | Slightly | Opalescent | Opalescent | Opalescent |
| Ph. Eur. 2.2.1 | Opalescent | Opalescent | (≤Reference | (≤Reference | (≤Reference |
| | to Very | (≤Reference | Suspension | Suspension | Suspension |
| | Opalescent ≤ | Suspension | III) | III) | III) |
| | Reference | II) | | | |
| | suspension IV) | | | | |
| Color, | Slightly Yellow | Yellow | Slightly | Slightly | Yellow |
| Ph. Eur. 2.2.2 | to Yellow | (<Y3) | Yellow | Yellow | (<Y3) |
| | (≤Y3) | | (<Y4) | (<Y4) | |
| Peptide | Complies with | Complies | Complies | Complies | Complies |
| Mapping or | reference | with | with | with | with |
| iCIEF | standard | reference | reference | reference | reference |
| | | standard | standard | standard | standard |
| pH, | 5.2-5.7 | 5.4 | 5.5 | 5.6 | 5.6 |
| Ph. Eur. 2.2.3 | | | | | |
| Osmolality | 400-500 | 442 | 436 | 447 | 458 |
| | mOsm/kg | | | | |
| Imaged | pI, 9.3-9.5 | 9.3 | 9.3 | 9.3 | 9.3 |
| Capillary | | | | | |
| Isoelectric | % Acidic | 29.0 | 27.1 | 27.3 | 28.0 |
| Focusing | Peaks, ≤32.0% | | | | |
| | % Main | 47.0 | 49.1 | 52.6 | 48.7 |
| | Peak, ≥40.0% | | | | |
| | % Basic | 24.0 | 23.7 | 20.1 | 23.4 |
| | Peaks, ≤40.0% | | | | |
| cGE - Reduced | Purity, ≥94.0% | 98.1 | 98.1 | 98.0 | 98.2 |
| | HC + LC | | | | |
| cGE - Non- | Purity, ≥85.0% | 93.5 | 94.9 | 95.3 | 94.0 |
| reduced | IgG | | | | |
| | ≤4.0% Light | 1.3 | 0.9 | 0.8 | 1.2 |
| | Chain | | | | |
| | ≤3.0% HMWS | 0.2 | 0.2 | 0.2 | 0.2 |
| Size Exclusion | Monomer, ≥95.0% | 97.9 | 98.1 | 98.3 | 98.1 |
| Chromatography | Total Aggregates | 2.0 | 1.8 | 1.6 | 1.9 |
| | (Dimer + | | | | |
| | Aggregates), ≤5.0% | | | | |
| | Fragments, ≤1.6% | 0.1 | 0.1 | 0.1 | 0.1 |
| Residual Protein | ≤5 ng/mg | 0 | 0 | 0 | 0 |
| A^{a} | protein | | | | |
| Host Cell | ≤20 ng/mg | 6 | 4 | 5 | 6/4 |
| Protein by | protein | | | | |
| ELISA^{a} | | | | | |
| Residual DNA^{a} | ≤5 pg/mg | <0.08 | <0.08 | <0.08 | <0.08/<0.08 |
| | protein | | | | |
| Bacterial | ≤2.0 EU/mL | <0.1 | <1.0 | <1.0 | <1.0 |
| Endotoxins, | | | | | |
| Ph. Eur. 2.6.14 | | | | | |
| Bioburden, | TAMC: ≤1 | 0 | 0 | 0 | 0 |
| Ph. Eur. 2.6.12 | CFU/10 mL | | | | |
| | TYMC: ≤1 | 0 | 0 | 0 | 0 |
| | CFU/10 mL | | | | |
| Protein | 180-220 | 205 | 208 | 207 | 207 |
| Concentration | mg/mL | | | | |
| CDC Activity by | 74-127% | 96 | 95 | 119 | 117 |
| Cell-Based | | | | | |
| Bioassay | | | | | |
| ADCC Potency | 63-177% | 98 | 98 | 95 | 103 |
| Assay | | | | | |

^{a}Controlled in-process data
cGE, Capillary Gel Electrophoresis;
LC, light chain;
HMWS, high molecular weight species;
HCP, Host cell protein

### Example 9. Arg/Glu vs. Citrate Formulations of Ublituximab

Viscosity of the new Arginine/Glutamate formulation of Ublituximab was compared to a previously used Citrate formulation of the same (described in WO 2025/199155). In particular, the viscosity of Variant 1/2 Arginine/Glutamate formulation (138 mM Arginine and 204 mM Glutamate) was compared to Current HC Citrate formulation (25 mM Citrate, 154 mM NaCl). The results are shown in FIG. 7.

As shown in FIG. 7, as compared to the Citrate formulations, the Arginine/Glutamate formulations reduced viscosity. The reduced viscosity of the Arginine/Glutamate formulations enabled delivery of 400 mg doses of Ublituximab in a volume of 2 mL in autoinjectors (e.g., in 200 mg/mL solution).

### Example 10. Stability of Ublituximab Solution for Injection (200 mg/mL)

The stability data for ublituximab solution for injection (200 mg/mL), as described here, includes 3-month data for the supportive stability study of the drug product (Table 44). All available results met stability acceptance criteria, with no trend seen in stability indicating results. All stability results met the acceptance criteria at the intended storage condition. The shelf-life will be based on the complete 3-month dataset. The supportive stability studies support 6 months of shelf life for ublituximab solution for injection (200 mg/mL) at the intended storage condition of 2-8° C. The product should be protected from light. The shelf life may be extended based on additional data.

The maximum shelf-life after the extension should not be more than double, or more than twelve months longer than the period covered by real time stability data obtained with representative batch(es). Table 43 below provides data availability and the assignable shelf-life to support clinical studies. Future extensions will be submitted as non-substantial amendments.

TABLE 43
Data Availability and Assignable Shelf Life

| Available Stability | Maximum Assignable |
|---|---|
| Data (months) | Shelf-Life (months) |
| 3 | 6 |
| 6 | 12 |
| 12 | 24 |
| 24 | 36 |

### Clinical Stability Studies

One clinical batch has been placed on stability to support the intended storage of ublituximab solution for injection (200 mg/mL) (Table 44). The primary stability study was designed to evaluate the stability for ublituximab solution for injection (200 mg/mL) under intended storage condition of 2-8° C. (5±3° C.) as well as accelerated condition (25±2° C.). The stability testing schedules for the clinical batches are listed in Table 45.

The stability samples are filled into 2R glass vials fitted with 13 mm stopper and cap that represent the same container closure material for the final drug product presentation. The results demonstrate that the ublituximab solution for injection (200 mg/mL) batch met the release acceptance criteria.

TABLE 44
Summary of Clinical Ublituximab Solution for
Injection (200 mg/mL) Stability Studies

| | Drug | | | Available | |
|---|---|---|---|---|---|
| | Substance | Storage | | Data | |
| Batch | Type | Condition | | (Months) | Status |
| 218-003-001 | GMP | 2-8° | C. | Initial | Ongoing |
| 218-003-001 | GMP | 25° | C. | Initial | Ongoing |

TABLE 45
Clinical Stability Schedule for Drug Product

| | | 2-8° C. | | | 25° C. |
|---|---|---|---|---|---|
| Test | Initial | 6 M | 12 M | 24 M | 3 M |
| Appearance, | X | X | X | X | NT |
| Opalescence | | | | | |
| Appearance, Color | X | X | X | X | NT |
| pH | X | X | X | X | X |
| Osmolality | X | X | X | X | X |
| Protein Concentration | X | X | X | X | X |
| CCIT | NT | NT | NT | X | NT |
| Sterility | X | NT | NT | NT | NT |
| Visible Particulates | X | X | X | X | X |
| Particulate Matter | X | X | X | X | NT |
| SEC | X | X | X | X | X |
| iCIEF | X | X | X | X | X |
| CGE (reduced and | X | X | X | X | X |
| non-reduced) | | | | | |
| Polysorbate 80 | X | X | X | X | X |
| Content | | | | | |
| ADCC Activity | X | X | X | X | X |
| CDC Activity | X | X | X | X | X |

### Supportive Stability Studies

In addition to the clinical stability study, a supportive stability study for ublituximab solution for injection (200 mg/mL) is also being conducted to support the shelf-life determination (Table 46). The stability testing schedules for the supportive stability batch are listed in Table 47. The results demonstrate that the supportive ublituximab solution for injection (200 mg/mL) batch met the acceptance criteria at the intended long-term storage and accelerated conditions evaluated and is representative of the clinical formulation.

TABLE 46
Summary of Supportive Ublituximab Solution
for Injection (200 mg/mL) Stability Studies

| | | | Available | |
|---|---|---|---|---|
| Batch | Storage | | Data | |
| Number | Condition | | (Months) | Status |
| PB#1 | 2-8° | C. | 3 | Ongoing |
| PB#1 | 25° | C. | 3 | Ongoing |

TABLE 47
Supportive Stability Schedule

| | | 2-8° C. | | | | | | | 25° C. | | |
|---|---|---|---|---|---|---|---|---|---|---|---|
| Test | Initial | 2 W | 1 M | 3 M | 6 M | 12 M | 18 M | 24 M | 1 M | 3 M | 6 M |
| pH | X | X | X | X | X | X | X | X | X | X | X |
| Protein | X | X | X | X | X | X | X | X | X | X | X |
| Concentration | | | | | | | | | | | |
| Visible | X | X | X | X | X | X | X | X | X | X | X |
| Particulates | | | | | | | | | | | |
| Particulate | X | X | X | X | X | X | X | X | X | X | X |
| Matter | | | | | | | | | | | |
| SEC | X | X | X | X | X | X | X | X | X | X | X |
| iCIEF | X | X | X | X | X | X | X | X | X | X | X |
| CGE | X | X | X | X | X | X | X | X | X | X | X |
| (reduced | | | | | | | | | | | |
| and non- | | | | | | | | | | | |
| reduced) | | | | | | | | | | | |
| ADCC | X | NT | NT | X | X | X | NT | X | NT | X | X |
| Activity | | | | | | | | | | | |
| CDC | X | NT | NT | X | X | X | NT | X | NT | X | X |
| Activity | | | | | | | | | | | |

### Stability Data Discussion and Conclusion

Stability for ublituximab solution for injection (200 mg/mL) is being monitored throughout clinical development. A subset of batch release assays was selected for the stability study based on their stability indicating properties. During development the test method as well as acceptance criteria evolved.

The results demonstrate that the ublituximab solution for injection (200 mg/mL) batch on stability met the acceptance criteria at the intended storage condition to date. As shown in the trending graphs, minimum rate of changes are observed for purity by SEC and CGE, and charge profile by iCIEF, which demonstrate the stability of the drug product for the intended storage condition.

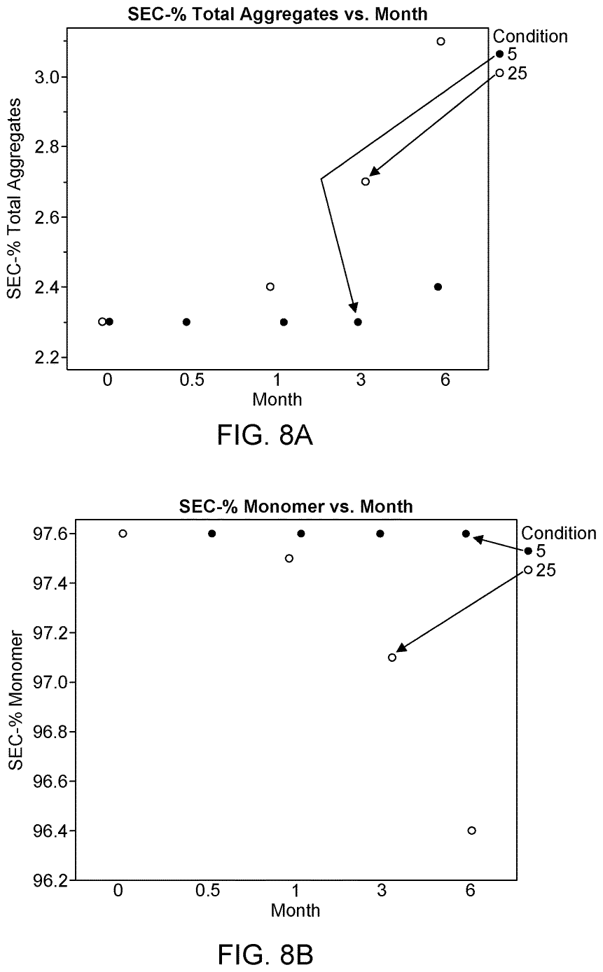

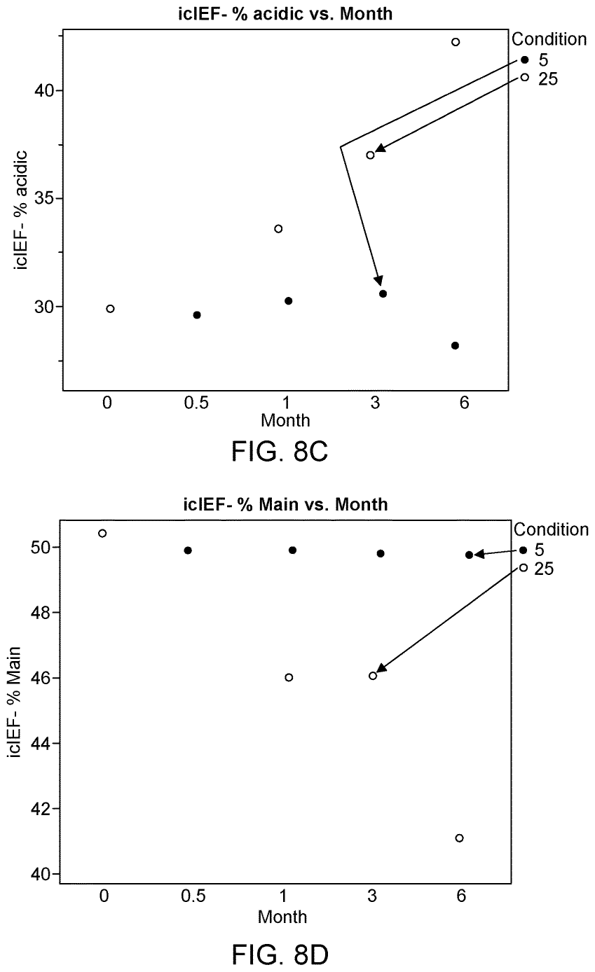

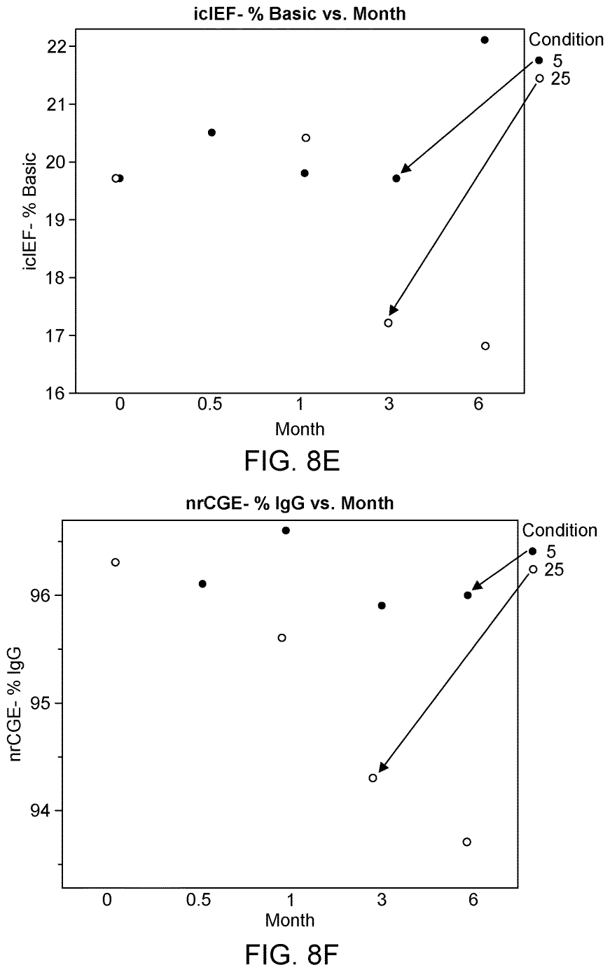

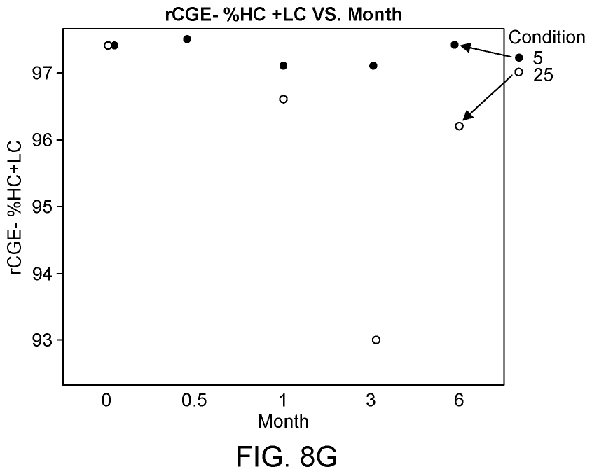

The stability for ublituximab solution for injection (200 mg/mL) was also studied under the accelerated condition of 25° C. As the data in Table 45 and Table 47, and the trending graphs in FIGS. 8A-8G show, no significant changes in product quality were observed for up to 3 months at the intended storage condition, demonstrating the stability of the drug product in the container closure system. Based on these data, an expiry of 6 months is assigned. The clinical stability studies are scheduled for up to 24 months.

### Example 11. Batch Analysis for Ublituximab Solution for Injection (200 mg/mL)

The drug substance batch numbers of ublituximab high concentration processed into drug product are summarized in Table 48. Batch analysis for the ublituximab solution for injection (200 mg/mL) batches are provided in Table 49. The batches met acceptance criteria.

TABLE 48
Lot Analysis Summary and Lineage

| Drug Substance | Drug Product | Batch Usage of |
|---|---|---|
| Batch Number | Lot Number | Drug Product |
| 2VAC2401CA | 218-003-001 | Stability/ |
| 2VAC2402CA | | Clinical |
| 2VAC2403CA | 218-003-002 | Clinical |
| 2VAC2404CA | 218-003-003 | Clinical |
| 3VAC2501CA | 218-003-004 | Clinical |
| 2VAC2503CA | 218-003-005 | Clinical |
| 2VAC2504CA | | |
| 2VAC2505CA | | |

TABLE 49
Batch Analysis Results for Drug Product

| | | Lot Number | | | | |
|---|---|---|---|---|---|---|
| | | 218-003- | 218-003- | 218-003- | 218-003- | 218-003- |
| | | 001 | 002 | 003 | 004 | 005 |
| | | Manufacturing Date | | | | |
| | | November | 3 Dec. | 9 Jan. | 4 Aug. | 19 Sep. |
| | | 2024 | 2024 | 2025 | 2025 | 2025 |
| | Acceptance | Batch Size | | | | |
| Test method | Criteria | 0.95 kg | 1.1 kg | 0.86 kg | 1.2 kg | 2.9 kg |
| Appearance, | Slightly | =Y4 | =Y4 | <Y4 | ≤Y5 | ≤Y5 |
| Color | yellow to | | | | | |
| Ph. Eur. 2.2.2 | yellow | | | | | |
| | (≤Y3) | | | | | |
| Appearance, | Slightly | =Reference | <Reference | <Reference | <Reference | <Reference |
| Opalescence | opalescent | suspension I | Suspension I | suspension II | Suspension III | Suspension II |
| Ph. Eur. 2.2.1, | to very | | | | | |
| USP<855> | opalescent | | | | | |
| | (≤Reference | | | | | |
| | suspension IV) | | | | | |
| Appearance, | Free of | Free of | Free of | Free of | Free of | Free of |
| Visible | visible | visible | visible | visible | visible | visible |
| Particulates | particulates | particulates | particulates | Particulates | particulates | particulates |
| Ph. Eur. 2.9.20, | | | | | | |
| USP<790> | | | | | | |
| pH | 5.2-5.7 | 5.3 | 5.3 | 5.3 | 5.4 | 5.3 |
| Ph. Eur. 2.2.3, | | | | | | |
| USP<791> | | | | | | |
| Osmolality | 400-500 | 431 | 438 | 443 | 421 | 429 |
| | mOsm/kg | | | | | |
| Protein | 180-220 | 193 | 199 | 199 | 197 | 208 |
| Concentration | mg/mL | | | | | |
| Peptide | Complies | Complies | Complies | Complies | Complies | Complies |
| Mapping or | with | | | | | |
| iCIEF | reference | | | | | |
| Extractable | 2.0-2.5 mL | 2.2 | 2.2 | 2.2 | 2.2 | 2.2 |
| Volume | | | | | | |
| Ph. Eur. 2.9.17, | | | | | | |
| USP<697> | | | | | | |
| Particulate | ≥25 μm: | 92 | 169 | 479 | 395 | 402 |
| Matter | ≤600 | | | | | |
| Ph. Eur. 2.9.19 | particles/vial | | | | | |
| Method 1 | ≥10 μm: | 131 | 369 | 490 | 531 | 444 |
| USP<787> | ≤6000 | | | | | |
| | particles/vial | | | | | |
| | ≥5 μm: | 153 | 181 | 219 | 257 | 213 |
| | Report result | | | | | |
| | ≥2 μm: | 297 | 331 | 313 | 313 | 302 |
| | Report result | | | | | |
| Bacterial | ≤2.0 | Beginning | Beginning | Beginning | Beginning | Beginning |
| Endotoxins | EU/mL | <0.5 EU/mL | <0.5 EU/mL | <0.5 EU/mL | <0.5 EU/mL | <0.5 EU/mL |
| Ph. Eur. 2.6.14, | | Middle | Middle | Middle | Middle | Middle |
| USP<85> | | <0.5 EU/mL | <0.5 EU/mL | <0.5 EU/mL | <0.5 EU/mL | <0.5 EU/mL |
| | | End | End | End | End | End |
| | | <0.5 EU/mL | <0.5 EU/mL | <0.5 EU/mL | <0.5 EU/mL | <0.5 EU/mL |
| Sterility | No growth | No growth | No growth | No growth | No growth | No growth |
| Ph. Eur. 2.6.1, | | | | | | |
| USP<71> | | | | | | |
| Capillary Gel | ≥94.0% | 97.8 | 97.5 | 97.8 | 98 | 97.9 |
| Electrophoresis | HC + LC | | | | | |
| (Reduced) | | | | | | |
| Capillary Gel | ≥85.0% IgG | 92.2 | 95.6 | 95.4 | 93.4 | 95 |
| Electrophoresis | ≤3.0% | 0.7 | 0.8 | 0.7 | 0.3 | 0.3 |
| (Non-reduced) | HMWS | | | | | |
| | ≤4.0% | 1.3 | 0.7 | 0.7 | 1.3 | 0.9 |
| | Light Chain | | | | | |
| Imaged | pI main = | 9.3 | 9.3 | 9.3 | 9.3 | 9.3 |
| Capillary | 9.3-9.5 | | | | | |
| Isoelectric | % Acidic | 26.0 | 25.8 | 25.3 | 27.8 | 27.1 |
| Focusing | peaks | | | | | |
| | ≤32.0% | | | | | |
| | % Main | 49.6 | 53.0 | 52.8 | 51.7 | 52.7 |
| | peak | | | | | |
| | ≥40.0% | | | | | |
| | % Basic | 24.3 | 21.1 | 21.8 | 20.5 | 20.3 |
| | peaks | | | | | |
| | ≤40.0% | | | | | |
| Size Exclusion | % Monomer, | 97.2 | 97.8 | 97.8 | 98.1 | 98.1 |
| Chromatography | ≥95.0% | | | | | |
| | Total | 2.7 | 1.9 | 2.2 | 1.9 | 1.8 |
| | Aggregates | | | | | |
| | (% Dimer + | | | | | |
| | Aggregates), | | | | | |
| | ≤5.0% | | | | | |
| | % Fragments, | 0.1 | 0.1 | 0.1 | 0.1 | 0.1 |
| | ≤1.6% | | | | | |
| Polysorbate 80 | 310-742 | 438 | 440 | 484 | 493 | 603 |
| Content | μg/mL | | | | | |
| CDC Activity | 74-127% | 94 | 98 | 97 | 84 | 91 |
| by Cell-Based | | | | | | |
| Bioassay | | | | | | |
| ADCC Potency | 63-177% | 119 | 108 | 106 | 108 | 93 |
| Assay | | | | | | |

### Example 12. Analysis of Ublituximab Solution for Injection (200 mg/mL) Measurement of Viscosity for Ublituximab Solution for Injection (200 mg/mL)

### Method of Measurement

Instrument: DVNext Rheometer by AMETEK Brookfield with CPA-40Z spindle; Speeds (RPM): 1.5, 2.0, 2.5. Samples were bracketed with 29 cP standard (31.45 cP from CoA). About 500 μL of sample was used. Data for each sample was collected at 1.5, 2.0, 2.5 rpm, which corresponds to a 15-25% torque range for the spindle. Samples and standards were run consistently.

TABLE 50
Data range for different subcutaneous DP lots - range of 23-30 cP

| | Manufacturing | Average Viscosity, |
|---|---|---|
| DS Lot# | Site | All Levels (cP) |
| 2VAC2401CA | Process X | 26.15 |
| 2VAC2402CA | | 23.31 |
| 2VAC2403CA | | 27.53 |
| 2VAC2404CA | | 30.07 |
| 2VAC2503CA | | 23.96 |
| 2VAC2504CA | | 22.85 |
| 2VAC2505CA | | 22.92 |
| 2VAC2506CA | | 26.77 |
| 2VAC2507CA | | 26.07 |
| 2VAC2508CA | | 24.32 |
| 3VAC2509CA | | 28.50 |
| 656A25-01 (PPQ1) | Process Y | 26.48 |
| 656A25-02(PPQ2) | | 24.35 |
| 656A25-03(PPQ3) | | 25.75 |
| 656A25-04(PPQ4) | | 27.41 |

Analysis of Arginine and Glutamate in Ublituximab Solution for Injection (200 mg/mL)

### Method of Measurement for Arginine and Glutamate

Arginine and glutamate were measured by liquid chromatography with tandem mass spectrometry. See, Example 13.

### Range of Arginine and Glutamate Measured

Process X manufactured batches displayed a range of Arginine=166±8 mM, Glutamate=224±7 mM.

TABLE 51
Range of Arginine and Glutamate

| Sample | Arginine (mM) | Glutamate (mM) |
|---|---|---|
| PD Run #1 | 182 | 237 |
| PD Run #2 | 159 | 222 |
| PD Run #3 | 162 | 218 |
| 2VAC2401CA | 164 | 225 |
| 2VAC2402CA | 163 | 225 |
| 2VAC2403CA | 166 | 219 |
| Mean ± SD | 16 ± 8 | 224 ± 7 |

Analysis of PS80 excipients in Ublituximab Solution for Injection (200 mg/mL)

Polysorbate 80 Content in Ublituximab was determined by HPLC-CAD. See, Example 14. Data experience for the range of DS manufactured at Process Y and Process X were 370-631 μg/mL (0.04-0.06%).

TABLE 52
Range of PS80 Content

| Samples | Manufacturing Site | Lot# | PS80 (μg/mL) |
|---|---|---|---|
| Clinical drug | Y | 656A25-01 BDS | 525 |
| substance (DS) | | 656A25-02 BDS | 534 |
| | | 656A25-03 BDS | 559 |
| | | 656A25-04 BDS | 511 |
| | X | 2VAC2401CA | 479 |
| | | 2VAC2402CA | 422 |
| | | 2VAC2403CA | 468 |
| | | 2VAC2404CA | 370 |
| | | 3VAC2501CA | 410 |
| | | 3VAC2502CA | 390 |
| | | 2VAC2503CA | 573 |
| | | 2VAC2504CA | 591 |
| | | 2VAC2505CA | 631 |
| Clinical drug | Z | 218-003-001 | 543 |
| product (DP) | | 218-003-002 | 440 |
| | | 218-003-003 | 484 |
| | | 218-003-004 | 493 |
| | | 218-003-005 | 603 |

### Example 13. Quantitation of Arginine and Glutamic Acid Excipients

Provided herein is an analytical test method (ATM) to describe the procedure used for the quantification of Arginine and Glutamic acid excipients in the formulation buffer for the Ublituximab solution for injection (200 mg/mL) using liquid chromatography with tandem mass spectrometry.

### Solution Preparation

All solutions were scaled proportionally.

Mobile Phase A1 (95/5/0.3 (v/v) of ACN/Water/Formic Acid)

Measure 950 mL ACN using a graduated cylinder.

Measure 50 mL of Water (LCMS grade) using a graduated cylinder.

Transfer both solvents into an HPLC bottle, add 3.0 mL of formic acid and mix.

Label the bottle appropriately and assign a 3-month expiration and store at room temperature.

### Mobile Phase B1 (100 mM Ammonium Formate)

Add approximately 500 mL of Water (LCMS Grade) to a 1 L volumetric flask.

Add 10 mL of 5050 M Ammonium Formate to the flask.

QS (Quantum satis, i.e., bring a solution up to a specified volume) to the mark with Water (LCMS Grade), and then transfer to an HPLC bottle.

Label the bottle appropriately and assign a 3-month expiration and store at room temperature.

Mobile Phase A2, Wash, and Purge Solvents (50% (v/v) Water in ACN)

Measure 500 mL of Acetonitrile (ACN) using a 1 L measuring cylinder.

Measure 500 mL of water (LCMS grade) using a 1 L measuring cylinder.

Transfer both solvents to an appropriate container, mix and label bottle appropriately. Assign a 3-month expiration and store at room temperature.

### Seal Wash Solvent (10% Methanol)

Use a graduated cylinder to transfer 450 mL of water and 50 mL of methanol to a 500-mL bottle.

Transfer the solution to a glass bottle, replace the cap, and mix well by inverting approximately 10 times.

Assign a 3-month expiration and store at room temperature.

### 0.1 N Hydrochloric Acid Solution

Measure 90 mL of Water (LCMS grade) using a 100 mL measuring cylinder.

Transfer to a suitable container.

Add 10 mL of 1 N HCl and mix.

Label the bottle appropriately and assign an expiry of six months and store at room temperature.

### 1.35 M Trichloroacetic Acid Solution

Add 3900 μL of water to an appropriately sized container

Add 1100 μL of 6.12 M TCA solution, mix well.

Label appropriately and assign an expiry date of six months. Store at room temperature.

### Preparation of Amino Acid Stock Solutions (2.25 mM Arg, 2.45 mM Glu)

Prepare amino acid stock solutions in 0.1N HCl by using the general procedure below:

Weigh approximately 4 mg of the amino acid into an appropriately sized container. Record the weight accurately.

Add the appropriate volume of 0.1N HCl to reach the concentration shown in Table 53.

Mix thoroughly by vortexing. If necessary, sonicate until the amino acid is fully dissolved in solution. Label appropriately. Assign an expiry of 6 months. Store at 2-8° C.

Prepare separate Arg and Glu stocks for calibration standards and quality control (QC) samples.

### Preparation of Labeled Amino Acid Stock Solutions (112.5 mM 13C6 15N4 Arg, 61.25 mM 13C5 15N Glu)

Prepare ~2 mL of stock solution for each labeled amino acid in 0.1N HCl by using the procedure below:

Weigh approximately 50 mg of 13C615N4 Arg or 19 mg 13C515N Glu into an appropriately sized container. Record the weight accurately.

Add the appropriate volume of 0.1N HCl, to reach the concentration specified in Table 53.

Mix thoroughly by vortexing. If necessary, sonicate until the amino acid is fully dissolved in solution. Label appropriately. Assign an expiry of 6 months. Store at 2-8° C.

¹³C₆¹⁵N₄ Arg and ¹³C₅¹⁵N Glu stocks require only one preparation.

TABLE 53
Amino Acid Stock Target Concentrations

| | | Target Concentration | Target Concentration |
|---|---|---|---|
| Amino Acid | Formula Weight | (mM) | (mg/mL) |
| Arginine | 174.20 | 2.25 | 0.3920 |
| Glutamic Acid | 147.13 | 2.45 | 0.3605 |
| ¹³C₆¹⁵N₄ Arginine | 220.59 | 112.5 | 24.8164 |
| ¹³C₅¹⁵N Glutamic Acid | 153.09 | 61.25 | 9.3768 |

### Preparation of Mixed Amino Acid Stock Solution (MAA)

Prepare separate mixed amino acid stocks for calibration standards and QC samples as described below:

Add 800 μL of 0.1N HCl diluent to a suitable container.

Add 100 μL of both Arginine and Glutamic acid stocks to the diluent.

Mix thoroughly by vortexing. Label appropriately. Assign an expiry of 1 month. Store at 2-8° C.

### Preparation of Mixed Labeled Amino Acid (MLAA) Stock Solution

Prepare a mixed labeled amino acid stock solution as described below:

Add 960 μL of 0.1N HCl diluent to a suitable container.

Add 20 μL of 112.5 mM 13C6 15N4 Arg and 20 μL 61.25 mM 13C5 15N Glu stocks to the diluent.

Mix thoroughly by vortexing. Label appropriately. Assign an expiry of 6 months. Store at 2-8° C.

### Preparation of Internal Standard Working Stock Solution for Standards and QCs (ISWSS)

Prepare 10 mL of ISWSS in 0.1N HCL using the following procedure. Scale preparation as needed.

Add 9900 μL of 0.1 N HCl to an appropriately sized container.

Add 100 μL of MLAA stock solution to the container to achieve final concentrations of 22.5 μM 13C6 15N4 Arg and 12.25 μM 13C5 15N Glu.

Mix thoroughly by vortexing. Label appropriately. Assign an expiry of 6 months. Store at 2-8° C.

### Preparation of Internal Standard Spiking Solution for Samples (ISSS)

Prepare 1 mL of ISSS by combining:

500 μL of 112.5 mM 13C6 15N4 Arg

500 μL of 61.25 mM 13C5 15N Glu.

Mix thoroughly by vortexing. Label appropriately. Assign an expiry of 6 months. Store at 2-8° C.

### Preparation of Calibration Standards

Prepare the calibration standards from the calibration standard MAA stock solution, ISWSS, and 0.1N HCl. The pipetting scheme for the preparation of the calibration standards and their target concentrations are displayed in Table 54. Label the calibration solutions appropriately.

TABLE 54
Calibration Standards

| | MAA | 0.1N | | Total | | |
|---|---|---|---|---|---|---|
| Calibration | Stock | HCL | ISWSS | Volume | [Arg] | [Glu] |
| Standard | (μL) | (μL) | (μL) | (μL) | (uM) | (uM) |
| 150% | 150 | 350 | 500 | 1000 | 33.8 | 36.8 |
| 125% | 125 | 375 | 500 | 1000 | 28.1 | 30.6 |
| 100% | 100 | 400 | 500 | 1000 | 22.5 | 24.5 |
| 75% | 75 | 425 | 500 | 1000 | 16.9 | 18.4 |
| 50% | 50 | 450 | 500 | 1000 | 11.3 | 12.3 |
| Diluent Blank | — | 1000 | 500 | 1000 | 0.0 | 0.0 |
| Blank | — | 500 | 500 | 1000 | 0.0 | 0.0 |
| System Suitability | 100 | 400 | 500 | 1000 | 22.5 | 24.5 |

### Preparation of QC Samples

Prepare QC samples from the QC MAA stock solution, ISWSS, and 0.1N HCl. The pipetting scheme used for the preparation of the Quality Control Samples are displayed in Table 55 below.

TABLE 55
Quality Control Samples

| Quality | MAA | 0.1N | | Total | | |
|---|---|---|---|---|---|---|
| Control | Stock | HCL | ISWSS | Volume | [Arg] | [Glu] |
| Sample | (μL) | (μL) | (μL) | (μL) | (uM) | (uM) |
| QCH | 150 | 350 | 500 | 1000 | 33.8 | 36.8 |
| QCM | 100 | 400 | 500 | 1000 | 22.5 | 24.5 |
| QCL | 50 | 450 | 500 | 1000 | 11.3 | 12.3 |

### Sample Preparation

Prepare a formulation buffer control sample alongside product B formulation samples as described below. The expected starting concentrations of arginine and glutamic acid in the buffer control and the product b formulation samples are 225 mM and 245 mM respectively. Due to the high viscosity of DS samples, a positive displacement pipette is used for transferring the 10 μL volume.

Combine 10 μL of sample with 20 μL of ISSS and 70 μL of 0.1N HCl to a microcentrifuge tube.

Vortex mixture for 30 s.

Transfer 25 μL of sample mixture to a tube containing 225 μL of 10% TCA.

Vortex for 1 minute, followed by 10 min centrifugation at 4° C., 20,000 RCF.

Transfer 10 μL of supernatant into an HPLC vial containing 990 μL of 0.1N HCl, mix by briefly vortexing.

Place vials in 10° C. autosampler and submit for analysis.

### Instrument Setup

Use the parameters shown in Table 56 and Table 57 below to create an instrument method. Place selected mobile phase lines (A-D) in the mobile phases. Place Seal Wash in 10% Methanol (Seal Wash Solvent). Place Purge line and Wash lines in 50% acetonitrile (Purge and Wash Solvent).

TABLE 56
UPLC Parameters

| Parameter | Setting | |
|---|---|---|
| Instrument | Water's Acquity UPLC | |
| Column | Intrada Amino Acid 50 × 3 mm (P/N WAA32) | |
| Mobile Phase A1 | 95/5/0.3 ACN/Water/Formic Acid | |
| Mobile Phase B1 | 100 mM Ammonium Formate | |
| Mobile Phase A2, | 50% Acetonitrile | |
| Wash and Purge | | |
| Solvents | | |
| Seal Wash Solvent | 10% Methanol | |
| Flow Rate | 0.6 | mL/min |
| Run Time | 8 | minutes |
| Injection Volume | 3 | μL |
| Sample Temp | 10 ± 3° C. | |
| Column Temp | 35 ± 3° C. | |
| Detection | UV 210 | nm |
| UV sample rate | 20 | points/second |

| | Step | Time (min) | % MPA1 | % MPB1 | Curve |
|---|---|---|---|---|---|
| Gradient | 1 | 0.00 | 90.0 | 10.0 | N/A |
| | 2 | 1.00 | 90.0 | 10.0 | 6 |
| | 3 | 5.00 | 0.0 | 100.0 | 6 |
| | 4 | 7.00 | 0.0 | 100.0 | 6 |
| | 5 | 7.10 | 90.0 | 10.0 | 6 |
| | 6 | 8.00 | 90.0 | 10.0 | 6 |

TABLE 57
Xevo G2-XS QTof Parameters

| Parameter | Setting | |
|---|---|---|
| Source Type | ESI | |
| Polarity | Positive | |
| Analyzer mode | Resolution | |
| Capillary voltage | 0.50 | kV |
| Sampling cone voltage | 35 | V |
| Source temperature | 130° | C. |
| Desolvation temperature | 600° | C. |
| Cone gas | 50 | L/h |
| Desolvation gas | 1000 | L/h |

| | |
|---|---|
| Acquisition time | Custom, 1.0-6.9 min |

| | | |
|---|---|---|
| Scan time | 0.05 | s |

| | |
|---|---|
| Collision energy | Off (6.0 V) |
| Intelligent Data Capture Intensity | Low (5) |
| Threshold | |

| | | | | 13C6 15N |
|---|---|---|---|---|
| | | 13C6 15N4 | | Glutamic |
| Components | Arginine | Arginine | Glutamic Acid | Acid |
| MRM Transition | 175.1190 > | 185.1278 > | 148.0604 > | 154.0748 > |
| | 84.0443 | 75.0761 | 84.0443 | 89.0554 |
| Expected RT (min) | 2.0 | 2.0 | 6.0 | 6.0 |
| Cone (V) | 35 | 35 | 35 | 35 |
| Collision (V) | 10-15 V | 10-15 V | 25-30 V | 25-30 V |

| | |
|---|---|
| LockSpray correction options | Mode: Custom |
| | Single reference mass: m/z 716.45795 or 734.46852 |
| | Single reference charge state: 1 |
| | Interval: 0.5 min |
| | Sample time: 1.00 s |
| | Attenuation: Custom pDRE setting determined during setup |
| | Collision energy: 6 V |
| | Flow rate: Flow rate determined during setup |
| | Capillary voltage: 3.00 kV |
| | Cone voltage: 30 V |
| Events | Time initial: Flow state = Waste, Sample |
| | Time initial: Reservoir = B, Reference |
| | Time initial: Refill = Refill Always, Reference |
| | Time initial: Infusion = Start, Reference |
| | Time 1.5 min: Flow state = LC, Sample |
| | Time 6.9 min: Flow state = Waste, Sample |

### Sequence

Include 7 injections of Diluent Blank (0.1N HCl) in the beginning of the sequence, followed by a Blank (SIL-spiked) injection. Include three system suitability injections, followed by a Diluent Blank injection. Bracketing injections must be included after every 10 samples. Use the 200 μmol/μL standard for bracketing.

TABLE 58
Recommended Sequence

| | | Injection Volume |
|---|---|---|
| Injection | # of Injections | (μL) |
| Diluent Blank | 7 | 1 |
| Blank | 1 | 1 |
| System Suitability Injections | 3 | 1 |
| Diluent Blank | 1 | 1 |
| Calibration Standards (50-150%) | 1 each level | 1 |
| Diluent Blank | 1 | 1 |
| QC Standards (L-H) | 1 each level | 1 |
| Diluent Blank | 1 | 1 |
| Test Samples (up to 10) | 1 per sample | 1 |
| Diluent Blank | 1 | 1 |
| QC Standards (L-H) | 1 each level | 1 |
| Diluent Blank | 1 | 1 |
| Calibration Standards (50-150%) | 1 each level | 1 |
| Diluent Blank | 1 | 1 |
| System Suitability Injections | 3 | 1 |
| Diluent Blank | 1 | 1 |

Continue cycles of Bracketing Control, Samples (n = 10), then Bracketing Control until all samples have been injected.

### Post Analysis Procedure

Include injections at the end of the sequence to: wash the column with 100% mobile phase B for 60 minutes; and flush the column with 50/50 ACN/Water for 30 minutes. Omission of the post-analysis procedure does not invalidate the assay.

### Data Processing

Data processing is completed fully independently for each amino acid. Results for one, or both, amino acids can be processed and reported from each data set. Failure of system suitability, or other criteria, for one amino acid does not impact reportability of the other amino acid.

### Example 14. Determination of Polysorbate 80 Content

Provided herein is a method for determination of Polysorbate 80 content in the ublituximab solution for injection (200 mg/mL) by reversed-phase chromatography with CAD detection.

### Solution Preparation

All solutions were scaled proportionally.

### Mobile Phase A (2% Formic Acid in Water)

Add 20 mL of formic acid to 980 mL of water. Mix with stir bar until complete dissolution.

Filter solution through a 0.2 μm filter

Store at room temperature. Expiration time: 1 week

### Mobile Phase B (2% Formic Acid in 2-Propanol)

Add 20 mL of formic acid to 980 mL of 2-propanol. Mix with stir bar until complete dissolution.

Filter solution through a 0.2 μm filter.

Store at room temperature. Expiration time: 1 week.

### Needle Wash (50% Acetonitrile in Water)

Mix 25 mL of water and 25 mL of acetonitrile

Mix well

Store at room temperature. Expiration time: 1 month

### Column Storage Solution (50% Methanol in Water)

Mix 500 mL of water and 500 mL of methanol.

Mix well and sonicate.

Store at room temperature. Expiration time: 1 month

10 mg/mL PS80 Stock Solution

In a 25-mL volumetric flask, weigh out 250±12.5 mg of PS80.

QS to 25 mL volume using water. Add slowly along the side to prevent bubble formation. Mix thoroughly.

Store at 2-8° C. Expiration time: 1 week

1 mg/mL PS80, 25% ACN in Water Intermediate Stock Solution (IM STK)

Ensure PS80 is completely dissolved in the 10 mg/mL PS80 stock solution before proceeding.

Add 400 μL 10 mg/mL PS80 stock solution to an appropriate glass container

Add 1000 μL of ACN

Add 2600 μL of water. Vortex until homogenous.

Prepare fresh on day of use.

### Diluent (25% ACN in Water)

Combine 10 mL of ACN and 30 mL of water. Mix until homogenous

Store in a well-sealed glass bottle. Prepare fresh on day of use.

### Blank, Needle Wash, Standard, and Sample Preparation

### Blank Preparation

Place an aliquot of Diluent into an HPLC vial

### Needle Wash

Place an aliquot of Needle Wash into an HPLC vial

### Calibration Standard Prep

Prepare 7 levels of calibration standards according to the Table 59 below.

TABLE 59
Calibration Standard Prep

| Level | | | Target PS80 |
|---|---|---|---|
| (IM STK, μL) | 1 mg/mL PS80 | Diluent (μL) | (μg/mL) |
| 1 | 20 | 980 | 20 |
| 2 | 30 | 970 | 30 |
| 3 | 40 | 960 | 40 |
| 4 | 50 | 950 | 50 |
| 5 | 60 | 940 | 60 |
| 6 | 80 | 920 | 80 |
| 7 | 100 | 900 | 100 |

Store calibration standards at 2-8° C. Expiration time: 24 hours.

Calculate actual PS80 concentration (to be used for calibration curve calculation) for each level using Equation 1.

$$Actual PS80 concentration ( \frac{μg}{mL} ) = ( \frac{M \times 1000}{{V}_{T1}} ) \times ( \frac{{V}_{1}}{{V}_{T2}} ) \times ( \frac{{V}_{2}}{{V}_{T3}} ) \qquad Equation 1$$

Where:
M=mass (mg) of PS80 used in preparation of 10 mg/mL PS80 stock solution
V₁=volume (μL) of 10 mg/mL PS80 used in preparation of 1 mg/mL PS80 (IM STK) solution
V₂=volume (μL) of 1 mg/mL PS80 (IM STK) used in preparation of calibration standard
V_{T1}=total volume (mL) of 10 mg/mL PS80 stock solution prepared
V_{T2}=total volume (μL) of 1 mg/mL PS80 (IM STK) prepared
V_{T3}=total volume (μL) of calibration standard prepared

### System Suitability Test (SST) Solution

Use the calibration standard solution prepared at 50 μg/mL target concentration as the SST solution.

### Reference Standard and Test Sample Preparation

If the PS80 concentration in the test sample is expected to be out of the highest point of the calibration standard solution, the test sample can be pre diluted with water If a pre-dilution is performed, the final dilution factor shall be calculated in Excel to reflect this. Pull all samples to be tested and store on benchtop to ensure samples are equilibrated to room temperature. Due to higher viscosity of HC-B, the following sample handling instructions are to be followed:
a) Samples are equilibrated for at least 60 min at room temperature.
b) Samples are mixed well by gentle inversion or by pipetting up and down gently: tap the sides to collect the sample at the bottom of the tube.
c) If condensate or solution is observed on top or sides of the test sample vial, it is recommended to briefly spin down the vial on a benchtop centrifuge to ensure all contents are mixed to homogeneity.

Use reverse pipetting or a positive displacement pipette when preparing samples and sample dilutions. Use of a positive displacement pipette is strongly recommended for the initial transfer of the undiluted RS and test sample to ensure accurate volume is transferred.

Prepare reference standard and samples in duplicate.

Dilute the reference standard and test sample(s) 1:3 in ACN. For example, add 600 μL of ACN to 200 μL of RS or test sample.

Vortex samples at high setting for about 5 seconds.

Centrifuge for 20 minutes at 16,000 rcf.

Dilute the supernatant 1:2 in water.
a) Transfer supernatant to a new tube. Be careful to pull volume from the top of the tube, avoiding contact with the pellet.
b) Add water to the transferred material.
c) Mix by pipetting up and down.

For example, transfer 100 μL of supernatant and add 200 μL of water. (Final Dilution Factor: 12).

### HPLC Setup

TABLE 60
Operating Parameters
CAD Settings

| Control Evaporator Temperature | Yes |
|---|---|
| Control Evaporator Temperature: Nominal | Low |
| Evaporator Wait for Temp Ready | Yes ± 5.0K |
| Data collection rate | 10 |
| Filter | 3.6 |
| Power function | 1.0 |

HPLC Settings

| Sampler | Injection volume | 20 | μL |
|---|---|---|---|
| | Enable Needle Wash | Yes | |
| | Sample | 5.0 ± 3.0° C. | |
| | Temperature | | |
| Column | Column | 35.0 ± 5.0° C. | |
| Compartment | temperature | | |
| | Timetable | See Section 6.3.2 | |
| Pump | Flow Rate | 1.0 | mL/min |
| | Run Time | 10 | min |

| | Time (min) | Mobile Phase A (%) | Mobile Phase B (%) |
|---|---|---|---|
| Gradient | 0.0 | 90 | 10 |
| Program | 1.0 | 80 | 20 |
| | 3.4 | 80 | 20 |
| | 3.5 | 0 | 100 |
| | 5.0 | 0 | 100 |
| | 5.1 | 90 | 10 |
| | 10.0 | 90 | 10 |

### Sample Set Acquisition

Generate a method set for blank subtraction. Generate a method set for conditioning blanks and subtraction blank. Injections for the assay should be run in the following sequence:

TABLE 60
Sequence for Injections for Assay

| Sample | Number of Injections | Dilution |
|---|---|---|
| Conditioning (Blank) ¹ | ~10 | 1 |
| Blank (Subtraction) ² | 1 | 1 |
| System Suitability Blank ³ | 2 | 1 |
| SST Solution | 1 | 1 |
| 20 μg/mL Standard Solution | 1 | 1 |
| 30 μg/mL Standard Solution | 1 | 1 |
| 40 μg/mL Standard Solution | 1 | 1 |
| 50 μg/mL Standard Solution | 1 | 1 |
| 60 μg/mL Standard Solution | 1 | 1 |
| 80 μg/mL Standard Solution | 1 | 1 |
| 100 μg/mL Standard Solution | 1 | 1 |
| System Suitability Blank | 1 | 1 |
| Reference Standard Rep 1 | 2 | 12 |
| Reference Standard Rep 2 | 2 | 12 |
| Test Sample 1 Rep 1 | 2 | 12 ⁵ |
| Test Sample 1 Rep 2 | 2 | 12 ⁵ |
| Test Samples 2 - N ⁴ | 2 per replicate | 12 ⁵ |
| System Suitability Blank | 1 | 1 |
| SST Solution | 1 | 1 |

¹ Approximately ten conditioning run blanks will be run. However, they may be run separately as a conditioning run prior to the sample sequence.
² Ensure that the Blank (subtraction) injection is assigned the Label “B01” that matches the injection label for blank subtraction in the method set. Refer to section 6.4.1.3c
³ Check the “Blank” checkbox in the Blank Column for both blanks following the subtraction blank
⁴ A bracketing System Suitability Blank and SST Solution (50 μg/mL standard solution) should be run after every 6 RS and/or test sample replicates (after total of 12 injections) and at the end of the sequence. Bracketing blanks and SST Solution may be run after fewer than 12 injections if desired.
⁵ Adjust the dilution factor to match the actual final dilution factor of each sample if a pre-dilution was performed

### Data Analysis

Create an appropriate processing method to perform automatic integration. If there is an issue associated with the use of automatic integration, manual integration can be performed. Refer to Table 61 below for example integration parameters.

TABLE 61
Example Integration Parameters

| Integration Algorithm | Traditional |
|---|---|
| Peak Width | 150 seconds |
| Threshold | 2000 |
| Minimum Area | 0 |
| Minimum Height | 0 |
| Inhibit Integration | 0 to 3.8 and 6 to 10.0 minutes |
| Components | PS80 |
| X values | Concentration |
| Y value | Area |
| Fit | Log-Log Linear |

Generate a final Results Set based on the calibration curve for the concentration (μg/mL) of each calibration standard, plotting the log of PS80 peak area (y) vs. the log of PS80 concentration (x) in μg/mL. The coefficient of determination (R2) for the curve must be recorded to at least 2 decimal places. The % Deviation for each standard must be recorded to the nearest whole number. Record the USP signal-to-noise value of the 20 μg/mL Standard Solution to a whole number. Record the peak area of any peaks in the blanks if observed, recorded to maximum precision. Record the concentration of PS80 (μg/mL) in each test sample and SST injection to maximum precision. Record the PS80 peak area of all Calibration Curve and Test Sample injections to a whole number and to greatest precision. Record the PS80 peak area of all SST injections, recorded to maximum precision. Calculate the mean PS80 peak area of all SST injections and record to maximum precision. Calculate the mean concentration (μg/mL) from duplicate sample injections and record to maximum precision and to at least 4 decimal places. If peaks were observed in any blanks in the region where the PS80 peak elutes (~4.5 minutes), calculate the % area relative to the average PS80 peak area of the SST PS80 solution peak areas using Equation 2. Record to a whole number. Note that only the PS80 peak can be integrated in SST Solution injections for proper custom field calculation. Use unrounded values to calculate:
% Difference of concentrations from duplicate injections using Equation 3; record to a whole number;
% Difference between PS80 concentration (μg/mL, average of both injections) obtained from duplicate preparations of each sample using Equation 4; record to a whole number; and
Average of both replicates' PS80 concentration for reference standard and test samples; record to at least one decimal place.

$$Blank Peak \% Area Relative to SST = \frac{Blank Peak Area}{Average SST Solution PS80 Peak Area} \times 100 \% \qquad Equation 2$$

$$\% Difference = \frac{\vert {C}_{2} - {C}_{1} \vert}{Average of {C}_{1} and {C}_{2}} \times 100 \% \qquad Equation 3$$

Where:
C₁=Concentration of injection 1
C₂=Concentration of injection 2

$$\% Difference = \frac{\vert {C}_{2} - {C}_{1} \vert}{Average of {C}_{1} and {C}_{2}} \times 100 \% \qquad Equation 4$$

Where:
C₁=Concentration of replicate 1
C₂=Concentration of replicate 2

### System Suitability Criteria

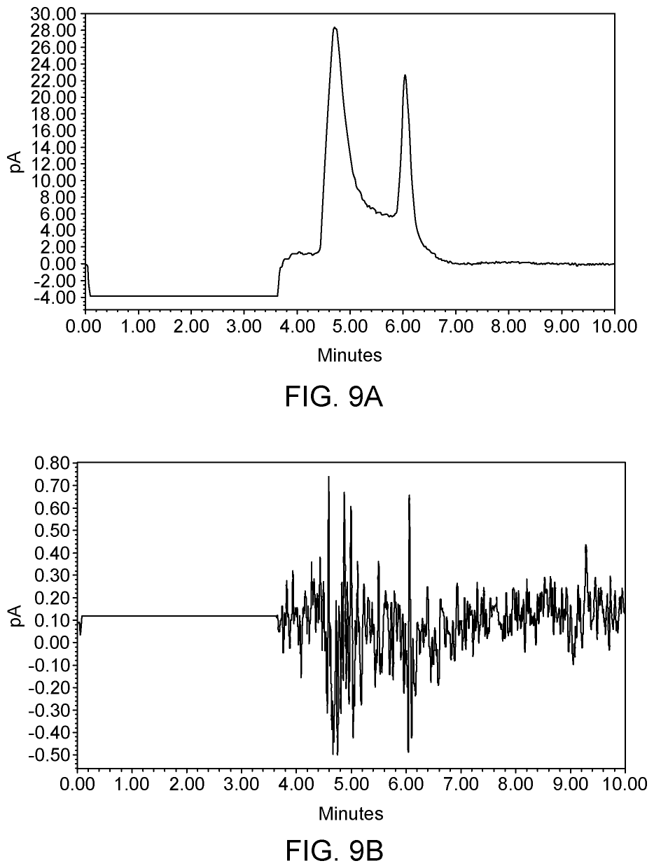

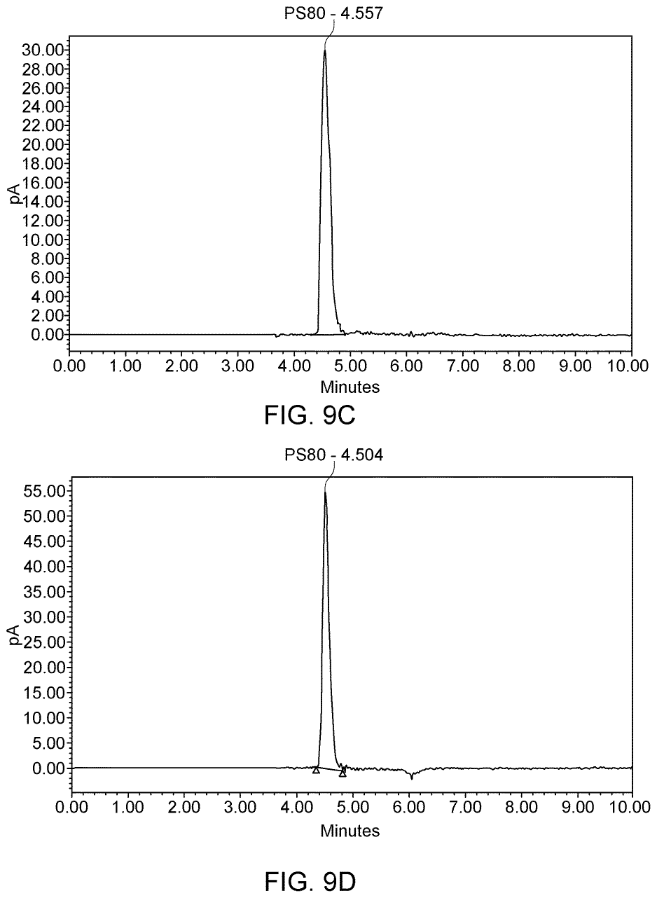

The blank-subtracted chromatograms corresponding to the system suitability injections must be visually comparable to the blank chromatogram shown in FIG. 9A, with no interfering peaks in the region where the PS80 peak elutes. All blanks injected later than the subtraction blank are considered system suitability injections. A peak is considered to be interfering if the peak area is greater than 10% of the SST Solution average peak area for all SST Solution injections (rounded to a whole number) and the signal-to-noise value for the peak is ≥3.

The coefficient of determination value (R2) for the calibration curve must be greater than or equal to 0.99. % Deviation of each standard must be ±12%. The USP signal-to-noise value for the 20 μg/mL Standard Solution must be ≥10.

PS80 concentration for every System Suitability Test (SST) injection must be in the range of 45 to 55 μg/mL.

### Ublituximab HC-B Reference Standard

Record the PS80 concentration result for the Reference Standard.

% Difference between PS80 concentration (μg/mL) obtained from duplicate injections must be less than or equal to 10%.

% Difference between PS80 concentration (μg/mL, average of both injections) obtained from duplicate preparations of a sample must be less than or equal to 12%.

### Sample Acceptance Criteria

% Difference between PS80 concentration (μg/mL) obtained from duplicate injections must be less than or equal to 10%. % Difference between PS80 concentration (μg/mL, average of both injections) obtained from duplicate preparations of a sample must be less than or equal to 12%. The PS80 peak area in each test sample injection must be between the PS80 peak area of the lowest (20 μg/mL) and highest (100 μg/mL) concentration Calibration Standards. If the measured peak area of PS80 in the test sample solution is out of range of the calibration standard solutions, the sample results are invalid. The assay will be repeated with an adjusted dilution factor by adjusting the water pre-dilution step. If the sample is analyzed without water pre-dilution and the measured peak area of PS80 in the test sample solution is lower than the lowest point of the calibration standard solutions, the result is not treated as invalid and should be reported.

### Example 15. Autoinjectors Comprising Ublituximab Solution for Injection (200 mg/mL)

Ublituximab solution for subcutaneous injection utilizes a ready-to-fill biologic drug substance at a target protein concentration of 200 mg/mL. The concentration allows a suitable volume fit for the primary packaging (the syringe) and the composition of buffering excipients achieves an appropriately low viscosity to enable dose delivery. The 2.25 mL staked-needle glass syringe, specifically the 27-gauge special thin wall needle, was chosen to facilitate dose delivery of the drug which has a viscosity of 25-30 centipoise. The fill volume was set to ensure a delivered dose of no less than 400 mg per 2.0 mL. The filled syringe was closed with a rubber plunger stopper to form an integral primary package, referred to as a staked-needle assembly (SNA).

SNAs were subsequently assembled into the SHL MOLLY® 2.25, a platform autoinjector that has a front sub-assembly paired with a rear sub-assembly. The front sub-assembly houses the SNA, a cap with a gripper to remove the rigid needle shield, and a clear viewing window. The rear sub-assembly or “power pack” provides the spring force mechanism, which depresses the plunger to expel the drug once activated. The MOLLY® 2.25 autoinjector is a robust device that, once assembled with the SNA, provides patients with an intuitive 2-step self-administration process. Through a robust design assessment and verification process, the device demonstrated the ability to deliver the 400 mg per 2.0 mL dose over 10 seconds once equilibrated to room temperature. This delivery performance was primarily enabled by the product composition, specifically the protein concentration and viscosity.

### Example 16. Pharmacokinetic/Pharmacodynamic Evaluation of Ublituximab in Patients with Autoimmune Diseases

Described herein is a Phase 1 study designed to evaluate the pharmacokinetics/pharmacodynamics (PK/PD) of ublituximab in two autoimmune disorders: Multiple Sclerosis (MS) and Myasthenia Gravis (MG).

### Rationale for the Study

Two identical, randomized, active-controlled studies (ULTIMATE I and ULTIMATE II) demonstrated significant efficacy outcomes of ublituximab versus teriflunomide in relapsing form of MS (RMS) (Steinman et al. Ublituximab versus Teriflunomide in Relapsing Multiple Sclerosis. N Engl J Med. 2022, 387(8):704-714). Particularly, ublituximab treatment was associated with a 54% relative reduction in annualized relapse rate (ARR) compared to teriflunomide, and approximately 96% and 91% relative reductions in T1 gadolinium (Gd)-enhancing lesions and new or enlarging T2 lesions (all significant), respectively. Based on these studies, the FDA and European Medicines Agency (EMA) approved administration schedule for ublituximab includes a 150 mg initial dose on Day 1 (4 hour IV infusion) followed by a 450 mg dose on Day 15 (1 hour IV infusion), and 450 mg (1 hour IV infusion) every 24 weeks thereafter (TG Therapeutics. Briumvi (ublituximab-xiiy) [Summary of Product Characteristics]. Published Jul. 13, 2023; TG Therapeutics. BRIUMVI (ublituximab-xiiy) [package insert]. Published Dec. 28, 2022).

In line with recent efforts to provide patients and healthcare providers with alternative options for drug delivery, a subcutaneous formulation of ublituximab has been developed. Subcutaneous delivery of monoclonal antibodies have proven to be a safe and effective option significantly alleviating burden on the healthcare system for infusion time and a preference for some patients (Bittner et al. Subcutaneous Administration of Biotherapeutics: An Overview of Current Challenges and Opportunities. BioDrugs. 2018, 32(5):425-440).

The current phase 1 study seeks to evaluate the bioavailability of ublituximab administered subcutaneously by evaluating PK, PD, tolerability, and safety. In Part 1 of the present study, participants in the subcutaneous (SC) cohorts received a single dose of ublituximab subcutaneously followed by subsequent doses of ublituximab administered intravenously in accordance with the approved labeling. In Part 2 of the present study, full SC regimens was evaluated with multiple doses of ublituximab administered subcutaneously in 8 or 12-week intervals following Day 1 and/or Day 15 subcutaneous doses.

To increase treatment options available to MG patients, this study also evaluated ublituximab administered to MG patients with moderate to severe muscle weakness. In addition to characterizing the PK and PD, safety and clinical activity in the MG study population was assessed by various clinical endpoints (e.g., MG-ADL).

### Part 1 Dose Selection

The current study leverages prior comparative bioavailability studies of monoclonal antibodies administered subcutaneously and intravenously. These studies demonstrated an approximate bioavailability of 50-85% for subcutaneous administration compared to an intravenous route of administration. For example, a recent Phase 3 study of a subcutaneous formulation of the anti-CD20 monoclonal antibody ocrelizumab demonstrated that 920 mg administered subcutaneously achieved similar exposure to 600 mg administered IV, representing ~65% relative bioavailability (Newsome et al. Subcutaneous Ocrelizumab in Patients With Multiple Sclerosis: Results of the Phase 3 OCARINA II Study. Neurology. 2025 May 13, 104(9):e213574). Therefore, subcutaneous doses between 195 mg and 300 mg were selected as potential candidates to match exposure of 150 mg ublituximab administered intravenously as they represent 77% and 50% bioavailability, respectively.

Prior to evaluating these target dose levels, and in order to establish tolerability of the subcutaneous route of administration, a starting dose of 50 mg was selected, which provides over a 10× fold margin of safety compared to the monkey No Observable Adverse Effect Level (NOAEL) of 50 mg/kg (equivalent to approximately 1130 mg in humans on a body surface area conversion) identified in a single-dose toxicity study using the subcutaneous route of administration in monkeys which was well tolerated. Further, assuming 50% bioavailability for the SC route of administration compared to the IV route of administration, the 50 mg dose level was selected as the dose that would result in 6-fold lower exposure compared to the approved 150 mg IV dose. Following successful clearance of the 50 mg and 100 mg dose levels using data collected from a minimum of 3 subjects, subsequent dose levels of 150 mg (SC3), 225 mg (SC4) and 300 mg (SC5) was evaluated. PK data was evaluated continually and additional intermediate dose levels were enrolled based on PK/PD and tolerability data review.

Lastly, while ublituximab dose levels below 195 mg administered subcutaneously are not expected to match the exposure of 150 mg administered IV, these initial dose levels were selected as doses that can produce a pharmacodynamic effect of attenuating peripheral B cells.

### Part 2 Dose Selection

The current approved therapeutic dose regimen of ublituximab is 450 mg administered intravenously every 24 weeks after a 150 mg dose on Day 1 and a 450 mg dose on Day 15. The dose level in Part 1 which were selected as pharmacokinetically equivalent to 150 mg administered intravenously was used as the dose administered initially on Day 1 in Part 2.

In the full SC regimen, SC doses administered every 12 weeks was evaluated to enable lower injection volumes that match the exposure (AUC) of 450 mg IV every 24 weeks. Assuming dose linearity and no accumulation, two 225 mg IV doses administered 12 weeks apart were selected as doses that can match the exposure of a single 450 mg IV dose across 24 weeks. Therefore, SC dose regimens determined to be bioequivalent to q12 week 225 mg IV are proposed for evaluation in Part 2. Assuming the bioavailability of subcutaneous ublituximab administration is between 50-85%, the model-informed bioequivalent dose administered every 12 weeks subcutaneously would fall between 265 and 450 mg. Generally, bioequivalence is established when the 90% CI of the geometric mean ratio between the reference regimen and the tested regimen falls between 0.8 to 1.25, which is the target for the doses selected in this study. Therefore, selection of doses is based off the anticipated overall exposure during the period of observation rather than matching individual doses.

In the study described herein, 400 mg SC was chosen for further evaluation in Part 2 dosed in a q12 week regimen starting at Week 12 after varying initial doses on Day 1 and/or Day 15. Additionally, a q8 week regimen can be evaluated based on emerging PK/PD data.

### Study Objectives of Part 1: Initial Dose SC Replacement PK/PD

### Primary Objectives

To assess the pharmacokinetics and relative bioavailability of IV and SC ublituximab
To assess the pharmacodynamics of IV and SC ublituximab

### Secondary Objectives

To assess the safety and tolerability of IV and SC ublituximab
To assess the clinical efficacy of ublituximab (MG patients only)

### Study Objectives of Part 2: Full Subcutaneous Regimen PK/PD

### Primary Objective

To assess the pharmacokinetics of ublituximab when administered subcutaneously

### Secondary Objectives

To assess the pharmacodynamics of ublituximab when administered subcutaneously
To assess the safety and tolerability of ublituximab when administered subcutaneously
To assess the clinical efficacy of ublituximab when administered subcutaneously

### Endpoints of Part 1: Initial Dose SC Replacement PK/PD

### Primary Endpoints

1. AUC_{(0-D14)} following Dose 1 SC or IV
2. Percentage of participants with CD19+ B cell counts≤10 cells/μL at Day 15 pre-dose

### Secondary Endpoints

1. Other PK parameters following Dose 1 and Dose 2
2. Percentage of patients with CD19+ B cell counts≤10 cells/μL at Week 24
3. Incidence of all grades and Grade ≥3 adverse events (AEs) during the 120-week study duration
4. Percentage of patients with all grades and Grade ≥3 injection/infusion related reactions (IRR; local or systemic) following SC or IV administration at Day 1 and IV administration on Day 15

### Additional Secondary Endpoints for MG Participants

1. Change from Baseline in MG-ADL total score at Week 24
2. Change from Baseline in QMG total score at Week 24
3. Change from Baseline in the Revised 15-Component Myasthenia Gravis Quality of Life (MG-QOL15r) at Week 24
4. Change from Baseline in the Myasthenia Gravis Composite (MGC) score at Week 24
5. Autoantibody levels AchR and MuSK at Week 24
6. Incidence of MG-related hospitalizations
7. Incidence of QMG score ≤4 AND a daily dose of prednisone of ≤10 mg AND no rescue treatment at Week 24 and Week 48

### Endpoints of Part 2: Full Subcutaneous Regimen PK/PD

### Primary Endpoint

1. AUC_{(0-W24)}

### Secondary Endpoints

1. Other PK parameters throughout the study
2. Percentage of participants with CD19+ B cell counts≤10 cells/μL at prespecified timepoints during the 120-week study duration
3. Incidence of all grades and Grade ≥3 adverse events (AEs) during the 120-week study duration
4. Percentage of participants with all grades and Grade ≥3 injection related reactions (local injection site reactions or systemic injection reactions)
   Additional Secondary Endpoints for Participants with RMS
1. The proportion of participants with no change or reduction in number of T1 Gd-enhancing lesions from baseline to Week 48
2. The proportion of participants free of T1 Gd-enhancing lesions at Week 48
   Additional Secondary Endpoints for Participants with MG
1. Change from Baseline in MG-ADL total score at Week 24
2. Change from Baseline in QMG total score at Week 24
3. Change from Baseline in the Revised 15-Component Myasthenia Gravis Quality of Life (MG-QOL15r) at Week 24
4. Change from Baseline in the Myasthenia Gravis Composite (MGC) score at Week 24
5. Autoantibody levels AchR and MuSK at Week 24
6. Incidence of MG-related hospitalizations
7. Incidence of QMG score ≤4 AND a daily dose of prednisone of ≤10 mg AND no rescue treatment at Week 24 and Week 48

### RMS Inclusion and Exclusion Criteria

### RMS Inclusion Criteria

MS participants must meet all of the following inclusion criteria to be eligible for participation in this study:
5. 18-65 years old
6. Diagnosis of RMS (2017 Revised McDonald criteria)
7. EDSS score ≤5.5 at screening
8. Neurologically stable for >30 days prior to Day 1
9. IgG>4.0 g/L at screening
10. Female participants of childbearing potential must consent to use an effective method of contraception from consent and for 6 months after the last dose of ublituximab.

### RMS Exclusion Criteria

MS participants who meet any of the following exclusion criteria are not eligible to be enrolled to this study:
1. Primary-progressive MS (PPMS) or inactive Secondary Progressive MS (SPMS)
2. Active chronic (or stable but treated with immune therapy) disease of the immune system other than RMS (e.g., rheumatoid arthritis, scleroderma, Sjögren's syndrome, Crohn's disease, ulcerative colitis, etc.) or immunodeficiency syndrome (hereditary immune deficiency, drug-induced immune deficiency, etc.)
3. Participants with CD19+ B-cell count below 40 cells/μL at screening
4. Participants who previously received anti-CD20 therapy and discontinued treatment due to safety reasons or lack of efficacy
5. Participants who previously received any approved therapy to treat RMS within 5 half-lives of the medication prior to Day 1; or who have ever received ublituximab, alemtuzumab, cladribine, cyclophosphamide, mitoxantrone, or daclizumab
6. Treatment with any investigational agent within 5 half-lives of the investigational drug prior to Day 1, or treatment with any experimental procedure for RMS (e.g., treatment for chronic cerebrospinal venous insufficiency)
7. History of life-threatening injection/infusion related reaction (IRR), hypersensitivity, or anaphylactic reaction with anti-CD20 therapy, components of ublituximab solution or pre-treatment medications
8. Current evidence or known history of clinically significant infection, including: chronic, recurrent, or ongoing active viral, bacterial, or fungal infectious disease requiring long term systemic treatment such as, but not limited to chronic urinary tract infection, chronic pulmonary infection with bronchiectasis, tuberculosis, or active hepatitis C virus (HCV)
9. History of serious opportunistic or atypical infections, including human immunodeficiency virus (HIV)
10. History of active hepatitis B virus (HBV) as evidenced by a detectable hepatitis B surface antigen (HBsAg) or positive hepatitis B core antibody (HBcAb), or chronic hepatitis C infection. Participants with positive hepatitis C virus antibody (HCV Ab) are eligible only if polymerase chain reaction (PCR) is negative for HCV ribonucleic acid (RNA)
11. History or evidence (clinical, radiological, or biomarker) of suspected or confirmed progressive multifocal leukoencephalopathy (PML)
12. Receipt of any live or live-attenuated vaccines (including vaccines for varicella-zoster virus or measles) within 4 weeks prior to first study drug administration
13. Participants exposed to corticosteroids (not including steroids used for premedication, topical steroids or inhaled steroids) received within 30 days prior to Day 1.
14. Participants requiring treatment with intravenous immunoglobulin (IVIG) for decreased immunoglobulins within the 12 months prior to Day 1
15. Any severe or uncontrolled medical condition that could affect the participant's ability to participate
16. Females who are pregnant or nursing
17. History of cancer except:
   a. If considered likely to be cured (with supporting documentation from the treating oncologist if possible)
   b. Is not being actively treated with anti-cancer therapy or radiotherapy and, in the opinion of the Investigator, is not likely to require treatment in the ensuing 3 years
   c. Considered to have low probability of recurrence (with supporting documentation from the treating oncologist if possible)
   d. Adequately treated and/or resolved basal or in situ squamous carcinomas of the skin are permitted
18. Unwillingness or inability to comply with study and/or follow-up procedures outlined in the protocol.

### MG Inclusion and Exclusion Criteria

### MG Inclusion Criteria

MG participants must meet all of the following inclusion criteria to be eligible for this study:
1. Oculobulbar, bulbar or generalized MG (gMG) ≥18 years of age at the time of signing the informed consent
2. Diagnosed with MG at least 6 months (180 days) prior to the date of signing the informed consent
3. Confirmation of eligibility by:
   i. Positive serologic test for anti-AChR Abs or anti-MuSK Abs as confirmed at screening,
AND
One of the following (either historical or during screening):
   a. Abnormal neuromuscular transmission test demonstrated by single-fiber electromyography or repetitive nerve stimulation
   b. Positive anticholinesterase test (e.g., edrophonium chloride test)
   c. Demonstrated improvement in MG signs on oral cholinesterase inhibitors, as assessed by the treating physician
4. Myasthenia Gravis Foundation of America Clinical Classification Class II to IV at screening
5. MG-ADL≥6 at screening
6. IgG>4.0 g/L at screening
7. Patients receiving treatment with ANY of the following must have been receiving treatment and on a stable dose for the time periods specified below prior to the date of the informed consent:
   a. Azathioprine (AZA): Must have been on AZA for ≥6 months (180 days) and have been on a stable dose for ≥2 months (60 days). Dose may not exceed 3 mg/kilogram (kg)/day.
   b. Immunosuppressive therapies (IST) (i.e., mycophenolate mofetil [MMF], methotrexate [MTX], cyclosporine [CYC], tacrolimus [TAC], or cyclophosphamide [CY]), must have been on the IST for ≥3 months (90 days) and have been on a stable dose for ≥1 month (30 days).
   c. Oral corticosteroids (i.e., prednisone), must have been on a stable dose for ≥4 weeks (28 days). Dose may not exceed 40 milligram (mg)/day or >80 mg over a 2-day period (or equivalent dose of other corticosteroids).
   d. A cholinesterase inhibitor (i.e., pyridostigmine), must have been on a stable dose for >2 weeks (14 days). Dose may not exceed 480 mg/day.

### MG Exclusion Criteria

MG participants are excluded from the study if any of the following criteria apply:
1. Participants with CD19+ B-cell count below 40 cells/μL at screening
2. Treatment with any investigational agent within 5 half-lives of the investigational drug prior to Day 1, or treatment with any experimental procedure
3. History of life-threatening injection/infusion related reaction (IRR), hypersensitivity, or anaphylactic reaction with anti-CD20 therapy, components of ublituximab solution or pre-treatment medications
4. Current evidence or known history of clinically significant infection, including: chronic, recurrent, or ongoing active viral, bacterial, or fungal infectious disease requiring long term systemic treatment such as, but not limited to chronic urinary tract infection, chronic pulmonary infection with bronchiectasis, tuberculosis, or active hepatitis C virus (HCV)
5. History of serious opportunistic or atypical infections, including human immunodeficiency virus (HIV)
6. History of active hepatitis B virus (HBV) as evidenced by a detectable hepatitis B surface antigen (HBsAg) or positive hepatitis B core antibody (HBcAb), or chronic hepatitis C infection. Participants with positive hepatitis C virus antibody (HCV Ab) are eligible only if polymerase chain reaction (PCR) is negative for HCV RNA
7. History or evidence (clinical, radiological, or biomarker) of suspected or confirmed progressive multifocal leukoencephalopathy (PML)
8. Receipt of any live or live-attenuated vaccines (including vaccines for varicella-zoster virus or measles) within 4 weeks prior to first study drug administration
9. Any severe or uncontrolled medical condition that could affect the participant's ability to participate
10. Females who are pregnant or nursing
11. Unwillingness or inability to comply with study and/or follow-up procedures outlined in the protocol.
12. History of cancer, including any active or untreated thymoma or history of thymic carcinoma or thymic malignancy, with the following exceptions:
   a. If considered likely to be cured (with supporting documentation from the treating oncologist if possible),
   b. Is not being actively treated with anti-cancer therapy or radiotherapy and, in the opinion of the Investigator, is not likely to require treatment in the ensuing 3 years,
   c. Considered to have low probability of recurrence (with supporting documentation from the treating oncologist if possible),
   d. Adequately treated and/or resolved basal or in situ squamous carcinomas of the skin are permitted
   e. Treated patients with history of thymoma other than thymic carcinoma corresponding to clinical stage 1 and 2 with no evidence of recurrence as defined by a recent negative imaging study (CT scan with IV contrast or MRI scan within 6 months of enrollment) are eligible for enrollment.
13. Muscle weakness affecting only ocular or periocular muscles (MGFA class I).
14. History of thymectomy, thymomectomy, or any thymic surgery within the 12 months prior to screening
15. Clinical features that, in the opinion of the Investigator, are consistent with MG crisis/exacerbation or Clinical Deterioration, at the time of the signing of the informed consent, or at any time prior to enrollment
16. Use of rituximab at any time (or any other anti-CD20 therapy)
17. Concurrent use of Complement 5a (C5a) inhibitors during the study, e.g., eculizumab
18. Concurrent use of Neonatal Fc receptor antagonists during the study, e.g., efgartigimod
19. Use of the following within the 4 weeks prior to Day 1:
   a. IVIG
   b. Plasma exchange (PE)
20. Current use of the following at the described dosages:
   a. Mycophenolate mofetil >3 grams/day or mycophenolic acid >1440 mg/day

### Study Design

### Overview of Study Design

Ublituximab is currently approved for patients with RMS as an IV only dosing regimen. This study evaluated the bioavailability and tolerability of ublituximab administered subcutaneously in patients with RMS and MG. The study had 2 Parts. Part 1 evaluated the PK/PD, safety and tolerability of a single subcutaneous dose of ublituximab through dose escalation followed by IV dosing. Part 2 evaluated the PK/PD, tolerability, safety, and efficacy of multiple subcutaneous doses of ublituximab administered every 12 weeks after the initial Day 1 and/or Day 15 doses.

### Study Design of Part 1

This is a 120-week, Phase 1 multi-center study designed to evaluate the PK, PD, safety, and tolerability of ublituximab in patients newly starting ublituximab treatment. The study explores efficacy endpoints for the enrolled MG participants. The study evaluates different cohorts of participants receiving an initial dose of either IV or SC ublituximab followed by subsequent doses of IV ublituximab. The dose of SC ublituximab was escalated and then refined to assess tolerability, PK and PD. Part 1 is described in detail in WO 2025/199155, the entire contents of which are incorporated herein by reference.

Participants were screened up to 28 days prior to the first dosing date (Day 1). Study (Part 1) duration is 120 weeks.

### Study Design of Part 2

This is a 120-week, Phase 1 multi-center study designed to evaluate the PK, PD, safety, tolerability, and efficacy of a fully subcutaneous dosing regimen of ublituximab in participants with RMS and MG who are newly starting ublituximab treatment.

Part 2 leveraged PK/PD and tolerability data generated in Part 1 to evaluate fully SC dose regimens of ublituximab that are expected to result in similar exposure across 24 weeks to the approved IV regimen or result in similar PD activity. Assuming dose linearity and no accumulation, two 225 mg IV doses administered 12 weeks apart are expected to match the exposure of a single 450 mg IV dose across 24 weeks. Therefore, an SC dosing regimen expected to be bioequivalent to 225 mg IV, administered every 12 weeks following the initial doses on Day 1 and Day 15, was evaluated in Part 2. Additionally, a second dosing regimen with a 8-week cadence was evaluated.

Per the dosing regimen schedule outlined in Table 62 below, in the 300 mg D1/400 mg D15 cohort, on Day 1, participants received an SC dose of ublituximab which is expected to be bioequivalent to 150 mg IV based on information obtained in Part 1. On Day 15, Week 12 and every 12 weeks thereafter up to Week 96, SC doses were approximately 1.5 times the dose administered on Day 1 and are expected to result in exposures comparable to 225 mg administered intravenously. Assuming the bioavailability of subcutaneous ublituximab administration is between 50-85%, the model-informed bioequivalent dose administered every 12 weeks subcutaneously falls between 265 and 450 mg. The exact dose was confirmed upon modeling of the PK data obtained in Part 1 and was communicated to sites upon opening of Part 2.

Alternative Day 1 and Day 15 regimens: Alternative Day 1/Day 15 regimens can be explored in Part 2 to evaluate the PK/PD of these regimens while maintaining a q12 week dosing cadence following the initial injections.

Alternate 8-week dosing regimens: A q8-week dosing regimen starting after the Day 1 and Day 15 doses can be explored in Part 2 to evaluate the PK/PD of this regimen.

Table 62 below identifies regimens that can be opened in addition to the 300 mg D1/400 mg D15 cohort. Not all cohorts can be opened to enrollment.

Up to 12 participants (RMS and MG combined) were enrolled to evaluate the PK of the proposed subcutaneous dosing regimen. Additional participants can be enrolled in an expansion cohort with reduced PK sampling to further evaluate safety and efficacy.

TABLE 62
Part 2 Study Design

| | | | | | Cohort Size for |
|---|---|---|---|---|---|
| Initial Dose | | | | | Safety and |
| Regimen | | Day 15 | | Cohort Size for | Efficacy |
| Cohort | Day 1(Dose 1) | (Dose 2) | Week 12-96 | PK Evaluation | Expansion |
| 300 mg D 1/ | 300 mg SC | 400 mg SC | 400 mg SC | Up to 12 | Up to 30 |
| 400 mg D 15 | (SC dose | (Model- | (Model- | participants | participants |
| | equivalent to | informed SC | informed SC | | |
| | 150 mg IV as | dose equivalent | dose every 12 | | |
| | identified in | to 225 mg IV) | weeks | | |
| | Part 1) | | equivalent to | | |
| | | | 225 mg IV) | | |

Alternative Initial Dose Regimen (Day 1/Day 15) Cohorts^{a}

| | | | | | |
|---|---|---|---|---|---|
| 400 mg D 1/ | 400 mg SC | — | 400 mg SC | Up to 12 | Up to 30 |
| 0 mg D 15 | | | | participants | participants |
| 800 mg D 1/ | 800 mg^{b} SC | — | 400 mg SC | Up to 12 | Up to 30 |
| 0 mg D 15^{b} | | | | participants | participants |
| 400 mg D 1/ | 400 mg SC | 400 mg SC | 400 mg SC | Up to 12 | Up to 30 |
| 400 mg D 15 | | | | participants | participants |

| | | | | | |
|---|---|---|---|---|---|
| | | | | | Cohort Size for |
| | | | | | Safety and |
| | | | | Cohort Size for | Efficacy |
| Dose Cohort | Day 1 | Day 15 | Week 8-96 | PK Evaluation | Expansion |

Alternative Dosing Cadence: q8 week regimen

| | | | | | |
|---|---|---|---|---|---|
| 400 mg q8 | 400 mg SC | 400 mg SC | 400 mg SC | Up to 12 | Up to 30 |
| week | | | (Every 8 weeks) | participants | participants |

^{a}Alternative Initial Dose Regimen and Dosing Cadence Cohorts are optional cohorts. The decision to open Cohorts can be at the Sponsor's discretion and can be communicated to the sites based on evolving PK and PD determinations from this study.
^{b}In the 800 mg D 1/0 mg D 15cohort, 800 mg can be administered as two separate 400 mg injections given at separate injection sites (see Section 8.1.4.2). In this cohort, following the Day 1 dosing of the first participant in the cohort, a 48-hour observation period to review safety and tolerability data can be utilized before dosing subsequent participants in the same cohort.

Participants were screened up to 28 days prior to the first dosing date (Day 1). Study duration is 120 weeks and includes PK, PD, efficacy and safety assessments throughout.

### Part 1 Participant Assignment

Part 1 participant assignment was not randomized but prioritized enrollment of both RMS and MG participants into the SC cohorts in a sequential manner starting with enrollment into the lowest dose level SC cohort (50 mg SC, Cohort SC1) and escalating to Cohort SC5 (300 mg SC). Subsequently, based on PK/PD modeling and tolerability data from Cohorts SC1-SC5, enrollment continued in Part 1 to the model-informed SC dose cohort.

Completion of enrollment to a specific SC dose level required treatment of at least the minimum number of participants specified as the Cohort Size for a respective cohort, and determination by the Sponsor that an acceptable level of tolerability was demonstrated to proceed to the next dose level. For each dose level, safety and PD was evaluated by the Sponsor prior to proceeding to the subsequent SC dose level. During the interim period between accruing the minimum number of patients to the current SC dose level and the completion of the evaluation period for the SC dose level, enrollment to SC cohorts was closed, and instead eligible RMS participants were enrolled to the IV cohort, until the IV cohort has been filled. In this manner, SC cohorts were enrolled sequentially through Cohort SC5 with intermittent enrollment to the IV cohort based on the availability of an SC cohort. Not all SC dose-levels were enrolled based on the Sponsor's assessment of tolerability, PK and PD data. MG participants were not enrolled into the IV cohort.

Following dosing of the first participant in Cohorts SC1 through SC5, a 48-hour observation period to review safety and tolerability data was utilized before dosing subsequent participants in the same cohort. Based on the Sponsor's assessment of tolerability, PK and PD data for the minimum number of patients to be evaluated at each SC dose level, a decision was made whether to enroll additional patients at the current SC dose level or to proceed to the next SC dose level.

After the escalation of cohorts SC1-SC5 and PK/PD modeling to simulate a subcutaneous dose bioequivalent to the 150 mg IV ublituximab dose, enrollment into a cohort at this dose level(s) was opened (if it was not achieved during the SC1 through SC5 dose escalation period). Once the minimum number of participants are enrolled to enable confirmation of PK, further enrollment at this dose was opened with a reduced PK sampling schedule. The additional enrollment of the reduced PK expansion cohort did not exceed 50 participants, and comprised of MG and RMS participants.

### Part 2 Participant Assignment

Enrollment for Part 2 commenced once the subcutaneous dose of ublituximab that is bioequivalent to 150 mg IV is identified and PK modeling and simulation from Part 1 has permitted the determination of the 225 mg equivalent dose. Both RMS and MG participants can into Part 2.

Cohorts with alternative Day 1 and/or Day 15 dosing regimens and a 8-week dosing cadence regimen were added to the Part 2 study design. These cohorts represent options that can be explored based on emerging PK/PD information in the study. Not all cohorts can be opened for enrollment. In all Part 2 cohorts, based on the Sponsor's assessment of tolerability, PK and PD data for the minimum number of patients to be evaluated in the PK evaluation cohort, a decision was made whether to enroll additional patients in the PK evaluation cohort or to proceed with the Safety and Efficacy expansion cohort.

### Ublituximab Packaging and Labeling

Ublituximab was provided to the study site in three formulations:
25 mg/mL (150 mg/6 mL in 10 mL single-use vial) solution for infusion for IV administration (WHITE flip-off seal) (this formulation was produced as described in detail in WO 2023/235764, the entire contents of which are incorporated herein by reference)
150 mg/mL (150 mg/l mL in 2 mL single-use vial) solution for injection for SC administration (BLUE flip-off seal) (this formulation was produced as described in detail in WO 2025/199155, the entire contents of which are incorporated herein by reference)
200 mg/mL (400 mg/2 mL in a 2 mL single-use vial) solution for injection for SC administration (RED flip-off seal) (this formulation is described herein)
   Ublituximab provided to the study site was labeled appropriately to distinguish between IV and SC administration, and the two SC formulations, in compliance with the applicable local regulations.
   Pre-Medications Prior to Treatment with Ublituximab

All participants treated with ublituximab as an IV infusion, and SC participants in SC1-SC5 and PK Evaluation Cohorts, require pre-medication. Pre-medications should be completed approximately 30 to 60 minutes prior to each dose of ublituximab and include, at minimum:
an antihistamine, preferably non-drowsy (e.g., cetirizine 10 mg or diphenhydramine 50 mg)
a corticosteroid (methylprednisolone 100-125 mg or dexamethasone 20 mg)
an antipyretic, such as acetaminophen (500-1000 mg).

For participants receiving ublituximab IV, pre-medications can be administered intravenously or orally. For SC patients in SC1-SC5 and PK Evaluation cohorts, pre-medications should be administered orally. For participants receiving ublituximab SC in the Part 1 and Part 2 Expansion cohorts, pre-medication should not be administered unless communicated or agreed by the Sponsor.

All pre-medications must be recorded as concomitant medications in the eCRF.

### Ublituximab IV Administration

Ublituximab IV should be administered intravenously through a dedicated line. No other treatment may be co-administered with ublituximab. Ublituximab should only be diluted in 250 mL 0.9% NaCl before use. No data are available for other solutions such as 5% dextrose and 5% mannitol. Ublituximab IV should not be administered as an IV push or bolus.

### Infusion Rates

Ublituximab IV should be administered only by slow infusion via intravenous route as a single administration. The total infusion time of ublituximab includes only enough flush to clear the IV tubing line of study drug.

### Ublituximab SC Administration

Ublituximab SC (150 mg/mL or 200 mg/mL) must be administered as a subcutaneous injection by the PI, TN, or a designee. Site of Administration: Lower abdomen at least 2 inches from navel. Ublituximab should be injected into the subcutaneous tissue of the lower abdomen. Do not administer in an area where there is bruising, redness, tenderness, hardness, or near a mole, scar or stretch marks. Injection site location must be recorded in the eCRF. Ublituximab should not be self-administered.

### Storage Instructions for the Prepared SC Syringe

Use the prepared SC syringe immediately and do not store.

### Preparation of Ublituximab SC

Ublituximab SC doses should be prepared and administered only by subcutaneous injection as a single administration, e.g., as described in Table 63. The vial should be allowed to reach room temperature prior to administration. Withdraw the required volume from the vial using the specified size sterile syringe. Immediately prior to the SC injection, change the needle to the size specified for the SC injection. Administer the SC injection at an approximate 45-degree angle slowly and evenly, without force. Do not massage or rub the injection site after administration.

Symptomatic local or systemic Injection/Infusion Related Reactions can occur despite pre-medication and can be treated at the discretion of the treating physician, with medications including but not limited to: antipyretics, corticosteroids, antihistamines, oxygen and bronchodilators. In case the referenced medications are not available, equivalents can be used at the Investigator's discretion. If these medications are administered intravenously, the IV line used for ublituximab should not be used. Available at the bedside prior to ublituximab administration will be medications and resuscitation equipment for the emergency management of Injection-relation reactions, Infusion related reactions, anaphylaxis, hypotension and/or respiratory distress per institution guidelines. Management in response to Injection-related reactions and Infusion related reactions depend on the severity. Infusion/Injection Related Reactions can occur for up to 24 hours after ublituximab administration.

TABLE 63
200 mg/mL (400 mg/2 mL in 2 mL single-use
vial) for SC injection (RED flip-top seal)

| | Total volume to | | Needle gauge for | Needle Length for |
|---|---|---|---|---|
| Dose | be injected | Syringe size | injection* | injection* |
| 300 mg | 1.5 mL | 3 mL | 27-29 G | 1/2″ to 5/8″ |
| | | | | (13-16 mm) |
| 400 mg | 2 mL | 3 mL | 27-29 G | 1/2″ to 5/8″ |
| | | | | (13-16 mm) |

| | |
|---|---|
| 800 mg | Administer as two separate 400 mg doses at two different abdominal sites |
| | consecutively spaced at least 2 inches apart and at least 2 inches from the navel |

*Any length sterile needle may be used to withdraw ublituximab from the vial.

### Statistical Considerations

### Sample Size and Power

The primary objectives of this study are to evaluate tolerability of SC dosing at different dose levels and characterize the PK and PD of ublituximab administered subcutaneously after a single dose (Part 1) and after multiple doses (Part 2). As such, this study is not powered to meet a statistical goal; rather the sample size enables sufficient PK and PD characterization to inform future development of ublituximab administered subcutaneously.

Given the low inter-individual variability in ublituximab PK at the approved IV regimen (AUC coefficient of variance=28%; Cₘₐₓ CV %=15%), sample sizes of 6-12 participants are expected to provide a reasonable characterization of ublituximab PK following single or multiple SC doses and to enable an exploratory comparison to PK of the IV route of the approved regimen. The proposed sample sizes for Part 1 and Part 2 are also generally consistent with similar studies intended to explore PK comparability of different dose routes for monoclonal antibodies. Additionally, the sample size for expansion cohorts to explore safety and efficacy with descriptive analysis is in line with Phase 1 studies. Participants in Part 1 who drop out prior to Day 15 may be replaced at Sponsor discretion. Participants in Part 2 who drop out prior to Week 24 may be replaced at Sponsor discretion.

### Analysis Populations

### Safety Population

The Safety Population will include all participants who received at least one dose of ublituximab.

### PK Population

The PK Population will include all participants who have at least one post-baseline PK sample.

### Efficacy Population

The Efficacy Population will include all participants in the Safety Population who provided baseline and at least one post-baseline efficacy assessment.

### Participant Demographics and Baseline Characteristics

Baseline demographic and clinical characteristics will be summarized as percentages for categorical variables and as mean, standard deviation (SD), median, minimum, and maximum for continuous measures.

### Pharmacokinetic Analyses

All participants who receive at least one dose of ublituximab and have at least one measurable ublituximab concentration with associated dosing time and dosing information, without major deviations, will be included in the PK analysis population.

For Part 1 and Part 2, pharmacokinetic measures including but not limited to AUC₀₋ₜ, Cₘₐₓ, Tₘₐₓ, Cₗₐₛₜ, AUCₗₐₛₜ, Tₗₐₛₜ, and C_{trough,t}, and SC bioavailability will be estimated as data permit via non-compartmental analyses and population PK modeling. In Part 1, geometric mean ratios of the primary study endpoint AUC_{0-14d} after dosing on Day 1 in SC treatment cohorts vs Cohort IV will be calculated along with the associated 90% confidence intervals (CI). Similar calculations may be conducted for Cₘₐₓ after dosing on Day 1. In Part 2, geometric mean ratios of the primary study endpoint AUC_{0-W24} after SC dosing compared to the approved IV regimen will be calculated along with the associated 90% confidence intervals (CI). Similar calculations may be conducted for Cₘₐₓ after dosing.

### Pharmacodynamic Analyses

Pharmacodynamic measures will be analyzed on all participants in the PK population.

For the following proportion endpoint, the number and percent of participants of interest will be calculated along with exact (Clopper-Pearson) two-sided 95% CI.

Proportion of participants with B-cells ≤10 cells/μL at all evaluated post-baseline timepoints.

For the following continuous endpoint, CD19+ B-cell counts, descriptive statistics (n, mean, SD, median, Q1, Q3, minimum, and maximum) will be provided for all evaluated timepoints as well as the changes from baseline.

### Safety Analyses

Safety will be analyzed on the Safety population, pooled overall and by cohort. Safety analyses will be primarily based on the incidence, seriousness, and severity of Aes (including local and systemic IRRs) and supplemented by clinically meaningful laboratory results, physical examination, and vital signs, as available.

Detailed information collected for each AE will be included in a listing: adverse event term, start/stop dates of the AE, seriousness, severity, relationship to study drug, action taken, and outcome. In addition, the number and percentage of participants experiencing at least one treatment-emergent AE will be tabulated by body system, preferred term, and CTCAE grade. Treatment-emergent Aes, drug-related Aes, adverse events of special interest, SAEs, Aes with a fatal outcome, and Aes leading to discontinuation/dose modification will be further summarized.

### Immunogenicity Analyses

All participants who received at least one ublituximab dose and had at least one post-baseline ADA sample, without major deviations will be included in the immunogenicity analysis population. Anti-ublituximab antibody measures (% Treatment Emergent Anti-drug antibodies (TE-ADA) positive participants, titer values, % nAb positive) will be described at each measured time point and by cohort. The impact of anti-ublituximab antibody development on the PK, PD and safety of ublituximab may be explored. Additional analyses of PK and PD data excluding participants positive for anti-ublituximab antibodies may be conducted as appropriate.

### RMS Efficacy Analyses

RMS efficacy endpoints will be analyzed on the Efficacy Population in Part 2. For the following proportion endpoints, the number and percent of participants of interest will be calculated, along with exact (Clopper-Pearson) two-sided 95% CI:
Proportion of participants with no change or reduction in number of T1 Gd-enhancing lesions from baseline to Week 48.
Proportion of participants free of T1 Gd-enhancing lesions at Week 48

### MG Efficacy Analyses

The MG efficacy endpoints will be analyzed on the Efficacy Populations in Part 1 and Part 2.

For all efficacy endpoints, number and percentage of participants will be provided for categorical variables and descriptive statistics for continuous variables, with a by-visit summary for the observed value and change from baseline as appropriate.

### Example 17. Evaluation of Bioavailability and Tolerability of Ublituximab Administered Subcutaneously in Patients with Multiple Sclerosis and Myasthenia Gravis

The subcutaneous administration of ublituximab is currently being evaluated in a Phase 1 study. A synopsis of the study is provided in Example 16. A summary of the results from participants treated with 200 mg/mL subcutaneous ublituximab (e.g., as described under Part 2 of the study in Example 16) are provided in this Example.

### Disposition and Demographics

As described in Table 64 below, twenty-two (22) participants have been enrolled in Part 2 of the study. Demographics of the participants are described in Table 65.

TABLE 64
Part 2 Enrollment Summary

| | | | | Number of |
|---|---|---|---|---|
| | | | | participants |
| | | | Maintenance Dose | enrolled and |
| Cohort | Day 1 Dose | Day 15 Dose | until Week 96 | treated to date |
| 400 mg D 1/400 mg D 15 | 400 mg SC | 400 mg SC | 400 mg SC every 12 | 12 |
| q12 week | | | weeks | |
| 400 mg D 1/400 mg D 15 | 400 mg SC | 400 mg SC | 400 mg SC every 8 | 10 |
| q8 week | | | weeks | |

TABLE 65
Part 2 Demographics of Participants

| | |
|---|---|
| 400 D 1/400 D 15/ | 400 SC D 1/400 SC D 15/ |
| 400 SC q12 W | 400 SC q8 W |
| n = 12 | n = 10 |

Age

| | | |
|---|---|---|
| Mean (SD) | 50 (18) | 55 (20) |
| Median (Q1, Q3) | 45 (37, 64) | 60 (48, 69) |

Gender

| | | |
|---|---|---|
| Female | 4 (33%) | 6 (60%) |
| Male | 8 (67%) | 4 (40%) |

BMI

| | | |
|---|---|---|
| Median (Q1, Q3) | 25.0 (21.8, 29.9) | 30.9 (28.2, 32.4) |

### Safety

Among the 22 participants treated, there were no injection related reactions (systemic) or injection site reactions (local) reported. The only adverse effect (AE) occurring in more than one participant was upper respiratory tract infection.

There have been no AEs leading to drug discontinuation, no AEs leading to death, and no pregnancies have been reported. All AEs reported to date have been Grade 1 or 2 in severity, with no Grade 3, 4 or 5 events observed.

### Pharmacodynamics

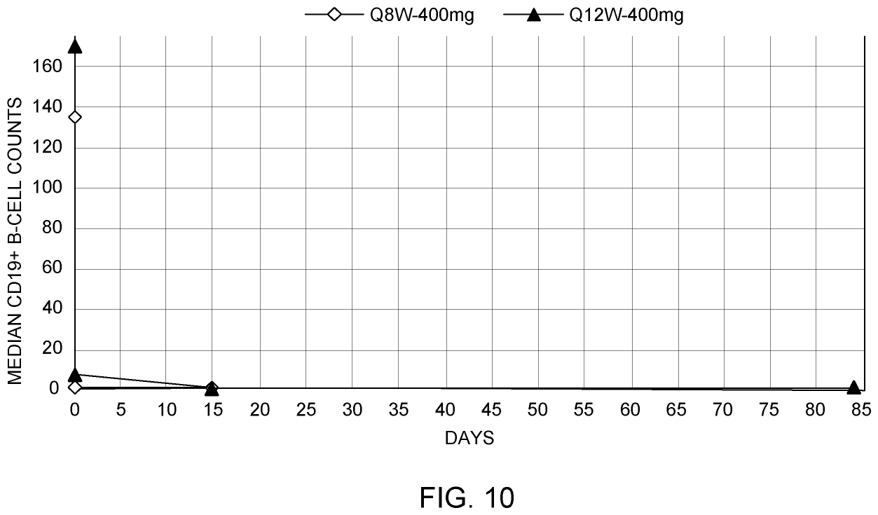

After a single dose, near complete depletion was observed within 3 hours of the dose and depletion of cells was maintained until the next dose (FIG. 10).

### Pharmacokinetics

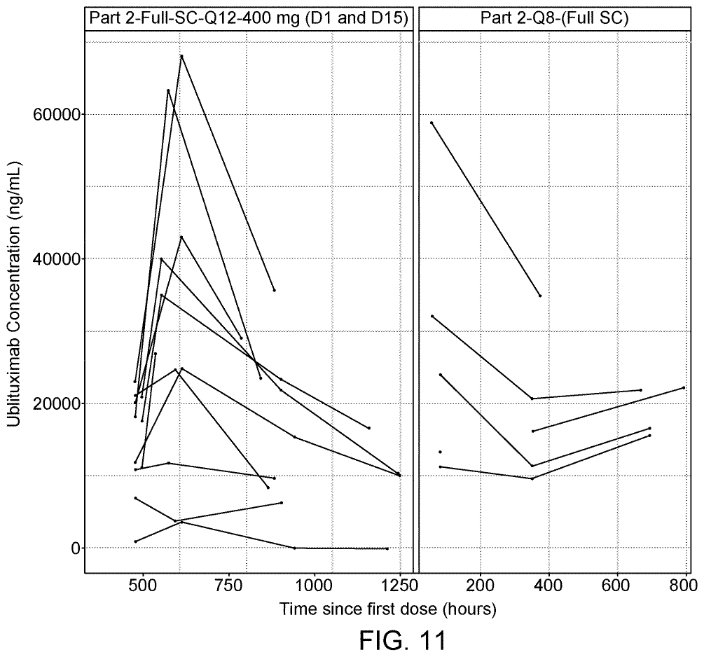

The Cₘₐₓ was significantly lower with SC administration when compared to IV administration. The ublituximab subcutaneous formulation described herein can have a bioavailability of about 50% to about 70% (e.g., about 55% to about 70%, about 60% to about 70%, about 65% to about 70%, about 55% to about 65%, or about 57% to about 63%) following SC administration. For example, the estimated bioavailability was about 61%. The simulated concentration versus time profiles (FIG. 11) followed the expected profile following subcutaneous administration.

Pharmacokinetic parameters as described here (e.g., pharmacokinetic parameters of the formulations of the present disclosure) show no significant difference over pharmacokinetic parameters of the formulations described in WO 2025/199155, the entire contents of which are incorporated herein by reference.

### REFERENCES

1. Hauser S L, Waubant E, Arnold D L, et al. B-Cell Depletion with Rituximab in Relapsing-Remitting Multiple Sclerosis. N Engl J Med. 2008; 358(7):676-688.
2. Hawker K, O'Connor P, Freedman M S, et al. Rituximab in patients with primary progressive multiple sclerosis: Results of a randomized double-blind placebo-controlled multicenter trial. Ann Neurol. 2009; 66(4):460-471.
3. Roche. Ocrevus (ocrelizumab) [Summary of Product Characteristics]. Published Apr. 13, 2023. Accessed Jan. 2, 2024.
4. Novartis. Kesimpta (ofatumumab) [Summary of Product Characteristics]. Published Mar. 1, 2023. Accessed Jan. 2, 2024.
5. Nowak R J, Coffey C S, Goldstein J M, et al. Phase 2 Trial of Rituximab in Acetylcholine Receptor Antibody-Positive Generalized Myasthenia Gravis: The BeatMG Study. Neurology. 2022; 98(4):e376-e389.
6. Tandan R, Hehir M K, Waheed W, Howard D B. Rituximab treatment of myasthenia gravis: A systematic review. Muscle Nerve. 2017; 56(2):185-196.
7. Piehl F, Eriksson-Dufva A, Budzianowska A, et al. Efficacy and Safety of Rituximab for New-Onset Generalized Myasthenia Gravis: The RINOMAX Randomized Clinical Trial. JAMA Neurol. 2022; 79(11):1105-1112.
8 Gilhus N E, Verschuuren J J. Myasthenia gravis: subgroup classification and therapeutic strategies. Lancet Neurol. 2015; 14(10):1023-1036.
9. Alvarez E, Steinman L, Fox E J, et al. Reduced Disease Progression With Ublituximab vs Teriflunomide in the Phase 3 ULTIMATE I and II Studies in Relapsing Multiple Sclerosis. In: CMSC.; 2022.
10. Steinman L, Fox E, Hartung H P, et al. Ublituximab versus Teriflunomide in Relapsing Multiple Sclerosis. N Engl J Med. 2022; 387(8):704-714.
11. Wallin M T, Culpepper W J, Nichols E, et al. Global, regional, and national burden of multiple sclerosis 1990-2016: a systematic analysis for the Global Burden of Disease Study 2016. Lancet Neurol. 2019; 18(3):269-285.
12. Parnell G P, Booth D R. The Multiple Sclerosis (MS) Genetic Risk Factors Indicate both Acquired and Innate Immune Cell Subsets Contribute to MS Pathogenesis and Identify Novel Therapeutic Opportunities. Front Immunol. 2017; 8.
13. Disanto G, Morahan J M, Barnett M H, Giovannoni G, Ramagopalan S V. The evidence for a role of B cells in multiple sclerosis. Neurology. 2012; 78(11):823-832.
14. Comi G, Bar-Or A, Lassmann H, et al. Role of B Cells in Multiple Sclerosis and Related Disorders. Ann Neurol. 2021; 89(1):13-23.
15. Gilhus N E. Myasthenia Gravis. N Engl J Med. 2016; 375(26):2570-2581.
16. Sanders D B, Wolfe G I, Benatar M, et al. International consensus guidance for management of myasthenia gravis. Neurology. 2016; 87(4):419-425.
17. De Romeuf C, Dutertre C A, Le Garff-Tavernier M, et al. Chronic lymphocytic leukaemia cells are efficiently killed by an anti-CD20 monoclonal antibody selected for improved engagement of FcγRIIIA/CD16. Br J Haematol. 2008; 140(6):635-643.
18. Esteves I T, Dumontet C, Herveau S, et al. LFB-R603 (Ublituximab), a third-generation monoclonal anti-CD20 antibody, displays additive antitumor activity with antileukemic chemotherapeutic agents in mouse xenograft models. In: ASH.; 2011.
19. Fox E, Lovett-Racke A E, Gormley M, et al. A phase 2 multicenter study of ublituximab, a novel glycoengineered anti-CD20 monoclonal antibody, in patients with relapsing forms of multiple sclerosis. Mult Scler J. 2021; 27(3):420-429.
20. TG Therapeutics. Briumvi (ublituximab-xiiy) [Summary of Product Characteristics]. Published Jul. 13, 2023. Accessed Jan. 2, 2024.
21. TG Therapeutics. BRIUMVI (ublituximab-xiiy) [package insert]. Published Dec. 28, 2022. Accessed Dec. 28, 2022.
22. Bittner B, Richter W, Schmidt J. Subcutaneous Administration of Biotherapeutics: An Overview of Current Challenges and Opportunities. BioDrugs. 2018; 32(5):425-440.
23. Newsome S D, Krzystanek E, Selmaj K W, Dufek M, Goldstick L, Pozzilli C, Figueiredo C, Townsend B, Kletzl H, Bortolami O, Zecevic D, Giacobino C, Clinch S, Shen Y A, Bhullar G D, Schneble H M, Centonze D. Subcutaneous Ocrelizumab in Patients With Multiple Sclerosis: Results of the Phase 3 OCARINA II Study. Neurology. 2025 May 13; 104(9):e213574.
24. Ameya U. Borwankar, Barton J. Dear, April Twu, Jessica J. Hung, Aileen K. Dinin, Brian K. Wilson, Jingyan Yue, Jennifer A. Maynard, Thomas M. Truskett, and Keith P. Johnston. Industrial & Engineering Chemistry Research 2016 55 (43), 11225-11234.
25. International Patent Application Publication Number WO 2025/078463 A1.
26. United States Patent Application Publication Number US 2023/0203179 A1.
27. Steven Ren. Current and emerging strategies for subcutaneous delivery of high-concentration and high-dose antibody therapeutics. Journal of Pharmaceutical Sciences, 2025 August; 114(8):103877.
28. Viola M, Sequeira J, Seiça R, Veiga F, Serra J, Santos A C, Ribeiro A J. Subcutaneous delivery of monoclonal antibodies: How do we get there? J Control Release. 2018 Sep. 28; 286:301-314.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical formulation comprising 100-300 mg/mL of an anti-CD20 antibody protein, 114 mM to 192 mM arginine, 184 mM to 242 mM glutamate, and 0.03% to 0.07% polysorbate 80,
wherein the anti-CD20 antibody protein comprises a heavy chain variable region (VH) comprising a VH complementarity determining region (CDR) 1 comprising the amino acid sequence of SEQ ID NO: 3, a VH CDR2 comprising the amino acid sequence of SEQ ID NO: 4, and a VH CDR3 comprising the amino acid sequence of SEQ ID NO: 5; and a light chain variable region (VL) comprising a VL CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10,
and wherein the anti-CD20 antibody protein has an N-glycan profile comprising about 10 to 20% galactosylated glycans and about 20 to 40% fucosylated glycans.

2. The pharmaceutical formulation of claim 1, wherein the pharmaceutical formulation comprises a concentration of about 200 mg/mL of the anti-CD20 antibody protein.

3. The pharmaceutical formulation of claim 1, wherein the anti-CD20 antibody protein comprises a heavy chain comprising the amino acid sequence of SEQ ID NO: 1 and a light chain comprising the amino acid sequence of SEQ ID NO: 2.

4. The pharmaceutical formulation of claim 1, wherein the pharmaceutical formulation comprises 127 mM to 179 mM arginine or 166±10% mM arginine.

5. The pharmaceutical formulation of claim 1, wherein the pharmaceutical formulation comprises 194 mM to 232 mM glutamate or 224±10% mM glutamate.

6. The pharmaceutical formulation of claim 1, wherein the pharmaceutical formulation comprises 0.045% to 0.055% polysorbate 80 or about 0.05% polysorbate 80.

7. The pharmaceutical formulation of claim 1, wherein pH of the pharmaceutical formulation is 4.95 to 6.05.

8. The pharmaceutical formulation of claim 1, wherein pH of the pharmaceutical formulation is about 5.5.

9. The pharmaceutical formulation of claim 1, wherein osmolality of the pharmaceutical formulation is 387 mOsm/kg to 473 mOsm/kg, optionally about 430 mOsm/kg.

10. The pharmaceutical formulation of claim 1, wherein viscosity of the pharmaceutical formulation is about 9-30 cP.

11. The pharmaceutical formulation of claim 1, wherein the pharmaceutical formulation is stable at 2-8° C. for up to 36 months.


SEQUENCE LISTING (50 sequences)

SEQ ID NO: 1 - protein, 448 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 7 aa, synthetic construct
GYTFTSY

SEQ ID NO: 4 - protein, 6 aa, synthetic construct
YPGNGD

SEQ ID NO: 5 - protein, 9 aa, synthetic construct
YDYNYAMDY

SEQ ID NO: 6 - protein, 118 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 7 - protein, 330 aa, synthetic construct [sequence omitted]

SEQ ID NO: 8 - protein, 10 aa, synthetic construct
RASSSVSYMH

SEQ ID NO: 9 - protein, 7 aa, synthetic construct
ATSNLAS

SEQ ID NO: 10 - protein, 9 aa, synthetic construct
QQWTFNPPT

SEQ ID NO: 11 - protein, 107 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKR

SEQ ID NO: 12 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, synthetic construct
  MOD_RES 1: note = pyroglutamate
EAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 213 aa, synthetic construct
  MOD_RES 1: note = pyroglutamate
EIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 447 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 4 aa, synthetic construct
MSCK

SEQ ID NO: 18 - protein, 15 aa, synthetic construct
ASGYTFTSYNMHWVK

SEQ ID NO: 19 - protein, 25 aa, synthetic construct
QTPRQGLEWIGGIYPGNGDTSYNQK

SEQ ID NO: 20 - protein, 9 aa, synthetic construct
GKATLTVGK

SEQ ID NO: 21 - protein, 48 aa, synthetic construct
SSSTAYMQLSSLTSEDSAVYFCARYDYNYAMDYWGQGTSVTVSSASTK

SEQ ID NO: 22 - protein, 58 aa, synthetic construct
DYFPEPVTVSWNSGALTSGVHTFPAVLQSSGLYSLSSVVTVPSSSLGTQTYICNVNHK

SEQ ID NO: 23 - protein, 28 aa, synthetic construct
PKDTLMISRTPEVTCVVVDVSHEDPEVK

SEQ ID NO: 24 - protein, 26 aa, synthetic construct
DTLMISRTPEVTCVVVDVSHEDPEVK

SEQ ID NO: 25 - protein, 14 aa, synthetic construct
FNWYVDGVEVHNAK

SEQ ID NO: 26 - protein, 12 aa, synthetic construct
VSNKALPAPIEK

SEQ ID NO: 27 - protein, 10 aa, synthetic construct
NQVSLTCLVK

SEQ ID NO: 28 - protein, 22 aa, synthetic construct
GFYPSDIAVEWESNGQPENNYK

SEQ ID NO: 29 - protein, 25 aa, synthetic construct
SRWQQGNVFSCSVMHEALHNHYTQK

SEQ ID NO: 30 - protein, 20 aa, synthetic construct
VTMTCRASSSVSYMHWYQQK

SEQ ID NO: 31 - protein, 19 aa, synthetic construct
SGTASVVCLLNNFYPREAK

SEQ ID NO: 32 - protein, 20 aa, synthetic construct
VDNALQSGNSQESVTEQDSK

SEQ ID NO: 33 - protein, 29 aa, synthetic construct
TKPREEQYNSTYRVVSVLTVLHQDWLNGK

SEQ ID NO: 34 - protein, 23 aa, synthetic construct
DIAVEWESNGQPENNYKTTPPVL

SEQ ID NO: 35 - DNA, 1344 bp, synthetic construct
  source 1..1344: mol_type = other DNA
caggcttatctacagcagtctggggctgagctggtgaggcctggggcctcagtgaagatgtcctgcaaggcttctggctacacatttaccagttacaatatgcactgggtaaagcagacacctagacagggcctggaatggattggaggtatttatccaggaaatggtgatacttcctacaatcagaagttcaagggcaaggccacactgactgtaggcaaatcctccagcacagcctacatgcagctcagcagcctgacatctgaagactctgcggtctatttctgtgcaagatatgactacaactatgctatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - DNA, 639 bp, synthetic construct
  source 1..639: mol_type = other DNA
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttattctttcacaatcagcagagtggaggctgaagatgctgccacttattactgccagcagtggacttttaacccacccacgttcggaggggggaccaggctggaaataaaacggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 37 - protein, 5 aa, synthetic construct
IRAHT

SEQ ID NO: 38 - protein, 4 aa, synthetic construct
EPAN

SEQ ID NO: 39 - protein, 19 aa, synthetic construct
QAYLQQSGAELVRPGASVK

SEQ ID NO: 40 - protein, 21 aa, synthetic construct
QGLEWIGGIYPGNGDTSYNQK

SEQ ID NO: 41 - protein, 24 aa, synthetic construct
YDYNYAMDYWGQGTSVTVSSASTK

SEQ ID NO: 42 - protein, 7 aa, synthetic construct
DTLMISR

SEQ ID NO: 43 - protein, 13 aa, synthetic construct
TKPREEQYNSTYR

SEQ ID NO: 44 - protein, 16 aa, synthetic construct
VVSVLTVLHQDWLNGK

SEQ ID NO: 45 - protein, 4 aa, synthetic construct
VSNK

SEQ ID NO: 46 - protein, 23 aa, synthetic construct
WQQGNVFSCSVMHEALHNHYTQK

SEQ ID NO: 47 - protein, 8 aa, synthetic construct
SLSLSPGK

SEQ ID NO: 48 - protein, 18 aa, synthetic construct
QIVLSQSPAILSASPGEK

SEQ ID NO: 49 - protein, 16 aa, synthetic construct
SGTASVVCLLNNFYPR

SEQ ID NO: 50 - protein, 26 aa, synthetic construct
VEAEDAATYYCQQWTFNPPTFGGGTR